(19) United States
(12) Patent Application Publication
(10) Pub. No.: US 2026/0240953 A1
(43) Pub. Date: Aug. 20, 2026

(54) PHARMACEUTICAL FORMULATION COMPRISING HETERODIMERIC RELAXIN FUSION PROTEINS AND USES THEREOF

(71) Applicant: ASTRAZENECA AB, Södertälje (SE)

(72) Inventor: Peng KE, Cambridge (GB)

(21) Appl. No.: 19/159,838

(22) PCT Filed: Mar. 1, 2024
(86) PCT No.: PCT/EP2024/055310

§ 371 Date: Aug. 26, 2025

(30) Foreign Application Priority Data
Mar. 3, 2023 (EP) 23160008.1

(51) Int. Cl. A61K 38/22; A61K 47/10; A61K 47/12; A61K 47/18; A61K 47/22; A61K 47/26

(52) U.S. Cl. CPC A61K 38/2221; A61K 47/10; A61K 47/12; A61K 47/183; A61K 47/22; A61K 47/26

(57) ABSTRACT

The present disclosure relates to pharmaceutical formulations comprising heterodimeric Relaxin fusion polypeptides, in particular heterodimeric Relaxin 2 fusion polypeptides and uses thereof. Thus, the disclosure provides pharmaceutical formulations comprising Relaxin fusion polypeptides and kits comprising the same and uses of the same including methods of treatment. The pharmaceutical formulations of the disclosure may be useful, in particular, in the treatment of cardiovascular diseases, for example for the treatment of heart failure.

### CROSS-REFERENCE TO RELATED APPLICATIONS

This application claims the benefit of priority of European Patent Application No. 23160008.1 filed 3 Mar. 2023, the contents of which are incorporated herein by reference.

### SEQUENCE LISTING

The present application contains a Sequence Listing which is hereby incorporated by reference in its entirety. The Sequence Listing, created on 16 Feb. 2024, is named 201258-WO-PCT Sequence listing.xml and is 82, 163 bytes in size.

### FIELD OF THE DISCLOSURE

The present disclosure relates to the field of pharmaceutical formulations. Specifically, a stable pharmaceutical formulation for a peptide-Fc fusion protein is provided.

### BACKGROUND

Biotherapeutics are a class of drugs in which the active ingredient has been derived from a biological source. As such, biotherapeutics include macromolecule therapeutics such as proteins, antibodies, peptides, and nucleic acids, as well as cell-based therapeutics. Thanks to their high specificity towards targets and superior safety profiles compared to small molecule therapeutics, biotherapeutics offer an effective and safe alternative to healthcare practitioners to treat a wide range of diseases and disorders. As such, biotherapeutics represent a rapidly growing portion of the therapeutics available to medical practitioners for treatment of a wide range of diseases and disorders.

However, the structural complexity and the size of biotherapeutics make them susceptible to instability in formulations. Biotherapeutics are susceptible to physical and/or chemical degradation, resulting in not only a reduction of efficacy and drug product shelf-life, but also a safety concern. Chemical degradation may encompass deamination, isomerization, oxidation, hydrolysis and glycation, whilst physical degradation can include aggregation, particle formation, precipitation, surface adsorption and denaturation. Both forms of degradation can negatively impact both the efficacy and the safety of biotherapeutics.

A well-designed manufacturing and purification process can often produce a high-purity product. However, many biotherapeutics can still degrade over time, during storage, transport, and administration. In some cases, the stability of the biotherapeutic is inherent to its molecular sequence. Whilst in some other cases, extrinsic factors, such as host cell proteins co-purified with the target biotherapeutic, or impurities from some excipients, can act as a “catalyst” to trigger either chemical or physical degradation.

Relaxin is a peptide hormone that belongs to the insulin superfamily. In humans, the Relaxin peptide family includes seven peptides of high structural but low sequence similarity: Relaxin-1, -2 and -3, and the insulin-like peptides INSL3, INSL4, INSL5 and INSL6. Naturally occurring Relaxins consist of A and B polypeptide chains covalently linked by two inter-chain disulphide bonds. The A chain has an additional intra-chain disulphide bond. In females, Relaxin-2 expression peaks during pregnancy, with Relaxin thought to play a role in placental development and fetal implantation. However, Relaxin has also been found to have anti-fibrotic properties. Heterodimeric fusions, e.g. HFUS1 (also termed RELAX0023), a recombinant fusion protein consisting of the Fc portion of human IgG1 connected to human Relaxin-2, have been shown to retain the anti-fibrotic effects of human Relaxin and consequently have been shown to exhibit efficacy as a therapy for heart failure, as described in WO2021/255127.

There remains a need to develop formulations of these heterodimeric fusion proteins having Relaxin activity, e.g. HFUS1, to meet their drug product shelf-life requirements.

### SUMMARY OF THE DISCLOSURE

During the formulation development of the heterodimeric fusions described herein, some challenges pertaining to the shelf-life and stability of the molecule were met. These included a tendency to self-associate, leading to aggregation; a tendency for amino acid clipping at certain pH ranges and a high susceptibility to form particles.

Accordingly, it is an object of the present disclosure to provide a stable formulation of a heterodimeric fusion having Relaxin activity, e.g. HFUS1, to meet its drug product shelf-life requirements and address one or more of the challenges described above.

Thus, the present disclosure relates to pharmaceutical formulations of heterodimeric fusions having Relaxin activity, e.g. HFUS1.

In one aspect, a pharmaceutical formulation is provided that comprises a heterodimeric fusion and a lipase-resistant surfactant, wherein the heterodimeric fusion comprises:
(i) a first heterodimerisation domain connected to at least one Relaxin A chain polypeptide or a variant thereof; and
(ii) a second heterodimerisation domain connected to at least one Relaxin B chain polypeptide or a variant thereof,
wherein the first heterodimerisation domain heterodimerises with the second heterodimerisation domain, and wherein the heterodimeric fusion has Relaxin activity.

In some embodiments, the Relaxin A chain and the Relaxin B chain are covalently bound by one or more (e.g. two) inter-chain bonds, optionally one or more (e.g. two) inter-chain disulphide bonds. In some embodiments, the Relaxin A chain and the Relaxin B chain are not covalently linked to each other by an amino acid linker.

In some embodiments, the Relaxin A chain is a Relaxin-2 A chain and the Relaxin B chain is a Relaxin-2 B chain.

In particular embodiments, the first and second heterodimerisation domains are derived from an immunoglobulin Fc region, e.g. an immunoglobulin G (IgG) Fc region, (“first Fc region” and “second Fc region”). The first and second Fc regions may comprise constant domains CH2 and/or CH3. In particular embodiments, the first and second Fc regions comprise CH2 and CH3.

In alternative embodiments, the first and second heterodimerisation domains are derived from an immunoglobulin Fab region.

In yet further alternative embodiments, the first and second heterodimerisation domains heterodimerise to form parallel coiled coils.

In some embodiments, the Relaxin A chain is connected to the first heterodimerisation domain (e.g. first Fc region) via a connector and the Relaxin B chain is connected to the second heterodimerisation domain (e.g. second Fc region) via a connector. In particular embodiments, one or both connectors are polypeptides.

In some embodiments, at least one connector is a polypeptide having a length of between 6 and 40 amino acids. In particular embodiments, both connectors are polypeptides having a length of between 6 and 40 amino acids. In particular embodiments, at least one connector is a polypeptide having a length of 21 amino acids. In particular embodiments, both connectors are polypeptides having a length of 21 amino acids. In certain embodiments, both connectors have the sequence GGGGSGGGGSGGGGSGGGGGS [SEQ ID NO: 5].

In particular embodiments, the C-terminus of the first heterodimerisation domain (e.g. first Fc region) is connected to the N-terminus of the Relaxin A chain and the C-terminus of the second heterodimerisation domain (e.g. second Fc region) is connected to the N-terminus of the Relaxin B chain. In alternative embodiments, the N-terminus of the first heterodimerisation domain (e.g. first Fc region) is connected to the C-terminus of the Relaxin A chain and the N-terminus of the second heterodimerisation domain (e.g. second Fc region) is connected to the C-terminus of the Relaxin B chain.

In some embodiments, the first and second heterodimerisation domains (e.g. first and second Fc regions) comprise heterodimerisation-promoting amino acid mutations and/or modifications, which may be asymmetric heterodimerisation-promoting amino acid mutations and/or modifications. In particular embodiments, the heterodimerisation-promoting amino acid mutations are “Fc Knob” and “Fc Hole” mutations. In particular embodiments, the “Fc Knob” and “Fc Hole” mutations are present in the CH3 domains. In some embodiments, the first and second Fc regions are derived from a human IgG1 immunoglobulin, optionally wherein the C-terminal lysine (K447, according to the EU index as in Kabat) may be absent from the CH3 domain of the first and/or second Fc region. In particular embodiments, the first Fc region comprises “Fc Knob” mutations and the second Fc region comprises “Fc Hole” mutations. Alternatively, the first Fc region has “Fc Hole” mutations, and the second Fc region has “Fc Knob” mutations. In particular embodiments, the heterodimerisation-promoting amino acid mutations comprise “Fc Hole” mutations Y349C, T366S, L368A and Y407V, or conservative substitutions thereof, in one CH3 domain; and “Fc Knob” mutations S354C and T366W, or conservative substitutions thereof, in the other CH3 domain, wherein the amino acid numbering is according to the EU index as in Kabat. In particular embodiments, the first and/or second Fc region comprises the amino acid mutations L234F, L235E, and P331S, wherein the amino acid numbering is according to the EU index as in Kabat.

In embodiments of any aspect of the disclosure, the Relaxin-2 A chain polypeptide comprises the sequence as set forth in of SEQ ID NO: 1 or a variant thereof and the Relaxin-2 B chain polypeptide comprises the sequence as set forth in SEQ ID NO: 2 or a variant thereof. In some embodiments, the Relaxin-2 A chain polypeptide comprises the amino acid mutation K9H, K17M or K171.

In some embodiments, both connectors have the sequence GGGGSGGGGSGGGGSGGGGGS [SEQ ID NO: 5].

Also provided by the present disclosure is a pharmaceutical formulation comprising a heterodimeric fusion and a lipase-resistant surfactant, wherein the heterodimeric fusion comprises:
(i) an FcX-con-A fusion polypeptide; and
(ii) an FcY-con-B fusion polypeptide,
wherein:
A is a Relaxin A chain or variant thereof, e.g. a Relaxin-2 A chain or variant thereof;
B is a Relaxin B chain or variant thereof, e.g. a Relaxin-2 B chain or variant thereof;
FcY is an immunoglobulin (e.g. IgG1) Fc region with “Fc Hole” amino acid mutations and/or modifications, optionally comprising a CH3 domain having the amino acid mutations Y349C:T366S:L368A:Y407V or conservative substitutions thereof;
FcX is an immunoglobulin (e.g. IgG1) Fc region with “Fc Knob” amino acid mutations and/or modifications, optionally comprising a CH3 domain having the amino acid mutations S354C:T366W or conservative substitutions thereof; and
con is a connector, e.g. a connector polypeptide, such as the sequence GGGGSGGGGSGGGGSGGGGGS [SEQ ID NO: 5],
wherein the amino acid numbering is according to the EU index as in Kabat, wherein FcX heterodimerises with FcY, and wherein the heterodimeric fusion has Relaxin activity.

In particular embodiments, the heterodimeric fusion comprises or consists of a fusion polypeptide with the amino acid sequence of SEQ ID NO: 11 and a fusion polypeptide with the amino acid sequence of SEQ ID NO: 20.

In some embodiments of any aspect of the disclosure, the heterodimeric fusion further comprises one or more Fabs, optionally wherein the heterodimeric fusion comprises one Fab linked to the N-terminus of the first heterodimerisation domain (e.g. first Fc region) and a second Fab linked to the N-terminus of the second heterodimerisation domain (e.g. second Fc region).

In some embodiments of any aspect of the disclosure, the heterodimeric fusion further comprises a second Relaxin A chain polypeptide or variant thereof connected to the N-terminus of the first heterodimerisation domain (e.g. first Fc region) and a second Relaxin B chain polypeptide or variant thereof connected to the N-terminus of the second heterodimerisation domain (e.g. second Fc region), optionally wherein the second Relaxin A chain is connected to the first heterodimerisation domain (e.g. first Fc region) via a connector polypeptide and the second Relaxin B chain is connected to the second heterodimerisation domain (e.g. second Fc region) via a connector polypeptide.

The present disclosure also provides a pharmaceutical formulation comprising a heterodimeric fusion and a lipase-resistant surfactant, wherein the heterodimeric fusion comprises:
(i) FcX-B-L-A and FcY, optionally FcY-B-L-A; or
(ii) FcY-B-L-A and FcX, optionally FcX-B-L-A;
wherein:
FcY is an immunoglobulin (e.g. IgG1) Fc region with “Fc Hole” amino acid mutations and/or modifications, optionally comprising a CH3 domain having the amino acid mutations Y349C:T366S:L368A:Y407V, or conservative substitutions thereof;
FcX is an immunoglobulin (e.g. IgG1) Fc region with “Fc Knob” amino acid mutations and/or modifications, optionally comprising a CH3 domain having the amino acid mutations S354C:T366W, or conservative substitutions thereof;
B is a Relaxin B chain or a variant thereof, e.g. a Relaxin-2 B chain or variant thereof; A is a Relaxin A chain or a variant thereof, e.g. a Relaxin-2 A chain or variant thereof; and L is a linker polypeptide, optionally with the amino acid sequence GGGSGGGSGG [SEQ ID NO: 60],
wherein the amino acid numbering is according to the EU index as in Kabat, wherein FcX heterodimerises with FcY, and wherein the heterodimeric fusion has Relaxin activity. Alternatively, the FcX and the FcY are non-Fc heterodimerisation domains as described herein. In some embodiments, the Relaxin B chain is connected to FcX and/or FcY via a connector, optionally a connector polypeptide having a length of between 6 and 40 amino acids, e.g. a length of 21 amino acids.

The present disclosure further provides a pharmaceutical formulation comprising a heterodimeric fusion and a lipase-resistant surfactant, wherein the heterodimeric fusion comprises:
(i) FcX-A-L-B and FcY, optionally FcY-A-L-B; or
(ii) FcY-A-L-B and FcX, optionally FcX-A-L-B;
wherein:
FcY is an immunoglobulin (e.g. IgG1) Fc region with “Fc Hole” amino acid mutations and/or modifications, optionally comprising a CH3 domain having the amino acid mutations Y349C:T366S:L368A:Y407V, or conservative substitutions thereof;
FcX is an immunoglobulin (e.g. IgG1) Fc region with “Fc Knob” amino acid mutations and/or modifications, optionally comprising a CH3 domain having the amino acid mutations S354C:T366W, or conservative substitutions thereof;
A is a Relaxin A chain or a variant thereof, e.g. a Relaxin-2 A chain or variant thereof;
B is a Relaxin B chain or a variant thereof, e.g. a Relaxin-2 B chain or variant thereof; and L is a linker polypeptide, optionally with the amino acid sequence GGGSGGGSGG [SEQ ID NO: 60],
wherein the amino acid numbering is according to the EU index as in Kabat, wherein FcX heterodimerises with FcY, and wherein the heterodimeric fusion has Relaxin activity. Alternatively, the FcX and the FcY are non-Fc heterodimerisation domains as described herein.

In some embodiments, the Relaxin A chain is connected to FcX and/or FcY via a connector, optionally a connector polypeptide having a length of between 6 and 40 amino acids, e.g. a length of 21 amino acids.

In some embodiments of any aspect of the disclosure, the ratio of Relaxin activity of the heterodimeric fusion over the Relaxin activity of a reference Relaxin protein is between about 0.001 and about 10.

In some embodiments of any aspect of the disclosure, the formulation comprises less than about 10,000, about 6000, about 5,000, about 1,000, about 750, about 600, about 500, about 250, about 150, about 100, or about 50 particles/mL greater than 2 μm, 5 μm, 10 μm, 15 μm, 20 μm or 25 μm diameter.

In some embodiments of any aspect of the disclosure, the concentration of the lipase-resistant surfactant is from 0.001% (w/v) to 1% (w/v). In certain embodiments, the concentration of the lipase-resistant surfactant is from 0.005% (w/v) to 0.2% (w/v). In certain embodiments, the concentration of the lipase-resistant surfactant is from 0.02% (w/v) to 0.06% (w/v). In particular embodiments, the concentration of the lipase-resistant surfactant is 0.04% (w/v).

In some embodiments of any aspects of the disclosure, the lipase-resistant surfactant cannot be enzymatically hydrolysed by lipoprotein lipase (LPL), Lipase 9, Phospholipase 2 or Phospholipase 2A. In particular embodiments, the lipase-resistant surfactant cannot be enzymatically hydrolysed by LPL. In some embodiments, the lipase-resistant surfactant does not comprise an ester bond capable of being enzymatically hydrolysed by lipoprotein lipase, Lipase 9, Phospholipase 2 or Phospholipase 2A. In some embodiments, the lipase-resistant surfactant does not comprise an ester bond capable of being enzymatically hydrolysed by lipoprotein lipase. In some embodiments, the lipase-resistant surfactant is a water-soluble non-ionic triblock copolymer formed by polyethylene oxide (PEO) and polypropylene oxide (PPO) blocks. In particular embodiments, the water-soluble nonionic triblock copolymer is poloxamer 188 (P188). Alternatively, the lipase-resistant surfactant is D-α-Tocopherol polyethylene glycol succinate (TPGS). In yet further embodiments, the lipase-resistant surfactant is selected from P188, TPGS, Kolliphor HS15, Kolliphor EL, Kolliphor RH40, PEG 300, PEG400, Brij 58 and Brij 35.

In some embodiments of any aspects of the disclosure, the formulation further comprises a buffer at a pH from about 3 to about 10, optionally about 5.5 to about 7.5. In particular embodiments, the formulation has a pH in the range of 6 to 7. In particular embodiments, the formulation has a pH of 6.5.

In some embodiments of any aspects of the disclosure, the concentration of the buffer is from 0.1 mM to 100 mM, such as 5 mM, 10 mM, 15 nM, 20 mM, 25 mM, 30 mM, 35 mM, 40 mM, 45 mM, or 50 mM. In particular embodiments, the concentration of the buffer is 10 mM to 30 mM. In particular embodiments, the concentration of the buffer is 20 mM.

In some embodiments, the buffer is selected from acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof. In particular embodiments, the buffer is selected from a citrate buffer and a histidine buffer. In particular embodiments, the buffer is a histidine, histidine hydrochloride or histidine/histidine hydrochloride buffer. In particular embodiments, the buffer is a histidine/histidine hydrochloride buffer (i.e. a combination of histidine and histidine hydrochloride). In one embodiment, the buffer is L-histidine/L-histidine hydrochloride monohydrate.

In some embodiments of any aspects of the disclosure, the formulation additionally comprises an excipient, optionally wherein the excipient is an ionic excipient. In some embodiments, the concentration of the excipient is from 100 mM to 300 mM. In particular embodiments, the concentration of the excipient is from 140 mM to 240 mM, e.g. 150 mM, 160 mM, 170 mM, 180 mM, 190 mM, 200 mM, 210 mM, 220 mM, 230 mM or 240 mM. In particular embodiments, the concentration of the excipient is 190 mM. Ionic excipients for use in the formulations described herein include salts and charged amino acids. The ionic excipient might comprise a combination of a salt and charged amino acid. Exemplary charged amino acids include arginine and lysine. Exemplary salts include chloride, succinate, acetate and sulfate salts, as well as carbonates, gluconates, lactates and malates. In particular embodiments, the ionic excipient is a charged amino acid hydrochloride (HCl) salt.

In some embodiments, the excipient is an ionic excipient selected from an arginine salt or a lysine salt. In some embodiments, the ionic excipient is selected from arginine HCl or lysine HCl. In particular embodiments, the ionic excipient is arginine HCl. It will also be understood that a buffer may, itself, be an ionic excipient as described herein. Thus, in some embodiments, the buffer is the ionic excipient.

In some embodiments of any aspects of the disclosure, the formulation further comprises a sugar, optionally wherein the sugar is sucrose. Other sugars that may be used include, but are not limited to, trehalose, lactose, mannitol, melibiose, melezitose, raffinose, mannotriose, stachyose, polyols such as trihydric or higher molecular weight sugar alcohols (e.g. glycerin, dextran, erythritol, glycerol, arabitol, xylitol, sorbitol, and mannitol), glucose, maltose, maltulose, iso-maltulose, lactulose and cyclodextrin.

In some embodiments of any aspects of the disclosure, the concentration of heterodimeric fusion is from 0.1 to 100 mg/mL, optionally 0.2 to 50 mg/mL, optionally 1 to 30 mg/mL. In some embodiments, the formulation comprises 0.2-50 mg/ml of heterodimeric fusion, 20 mM histidine/histidine hydrochloride buffer, 190 mM arginine HCl, 0.04% (w/v) poloxamer 188 (P188), and wherein the formulation has a pH of 6.5. In some embodiments, the formulation comprises 50 mg/ml of heterodimeric fusion. In other embodiments, the formulation comprises 30 mg/ml of heterodimeric fusion. In other embodiments, the formulation comprises 33 mg/ml of heterodimeric fusion. In other embodiments, the formulation comprises 5 mg/ml of heterodimeric fusion. In other embodiments, the formulation comprises 1 mg/mL of heterodimeric fusion. In other embodiments, the formulation comprises 1.1 mg/ml of heterodimeric fusion. In some embodiments, the heterodimeric fusion comprises or consists of a fusion polypeptide with the amino acid sequence of SEQ ID NO: 11 and a fusion polypeptide with the amino acid sequence of SEQ ID NO: 20.

In one aspect, the present disclosure further provides a pharmaceutical formulation as described herein for use in therapy. The present disclosure also provides a method of treating a subject with a disease or disorder, the method comprising administering the pharmaceutical formulation as described herein to the subject.

In one aspect, the present disclosure further provides a pharmaceutical formulation as described herein for use in the treatment of a subject with heart failure, including heart failure with pulmonary hypertension (e.g. Group 2 Pulmonary Hypertension). The present disclosure also provides a method of treating a subject with heart failure, including heart failure with pulmonary hypertension (e.g. Group 2 Pulmonary Hypertension), the method comprising administering the pharmaceutical formulation as described herein to the subject. In some embodiments, the heart failure is heart failure with reduced ejection fraction, heart failure with mid-range ejection fraction or heart failure with preserved ejection fraction. In some embodiments, the subject has a mean Pulmonary Arterial Pressure of about 25 mmHg or greater, a pulmonary artery wedge pressure (PAWP) greater than 15 mmHg and/or a Right Ventricular Systolic Pressure of about 40 mmHg or greater. In some embodiments, the subject has been fitted with a blood pressure monitoring device, which may be a pulmonary artery pressure monitoring device. In some embodiments, the pulmonary artery pressure monitoring device is a CardioMEMS pressure monitoring device. In some embodiments, the subject has a Pulmonary Vascular Resistance of less than 3.0 wood units. In other embodiments, the subject has a Pulmonary Vascular Resistance of 3.0 or more wood units.

In some embodiments, the fusion polypeptide or pharmaceutical formulation is suitable for and/or administered to the subject by subcutaneous injection. In some embodiments, the fusion polypeptide or pharmaceutical formulation is suitable for and/or administered by self-administration.

In some embodiments, administration of the pharmaceutical formulation results in one or more of reduced PVR; reduced mPAP; reduced ePAD; increased stroke volume (SV) of the heart; decreased systemic vascular resistance (SVR) and/or increase estimated glomerular filtration rate (eGFR); increased ejection fraction; and/or increased cardiac output; as compared to baseline levels pre-administration.

In one aspect, the present disclosure further provides a kit comprising the pharmaceutical formulation as described herein.

Aspects and embodiments of the disclosure are set out in the appended claims. These and other aspects and embodiments of the disclosure are also described herein.

### BRIEF DESCRIPTION OF THE DRAWINGS

FIG. 1 shows the purity loss of 50 mg/ml (A) and 10 mg/mL (B) HFUS1 after storage at 5° C., 25° C. and 40° C., for 3 months, 3 months and 1 month, respectively.

FIG. 2 shows the unfolding temperature profile of HFUS1, measured by differential scanning calorimetry thermogram.

FIG. 3 shows the self-diffusion coefficient and hydrodynamic radius of HFUS1 at a concentration of 0.005 g/mL, 0.008 g/mL, 0.011 g/mL, 0.016 g/mL and 0.020 g/mL.

FIG. 4A is a bar chart showing clipping of amino-acid at the C-terminus of chain B of relaxin of HFUS1 assessed by mass spectroscopy. F4, F5, F6 and F9 correspond to Formulations 4, 5, 6 and 9 respectively, as described in Table 4.

FIG. 4B is a bar chart showing change of % tri-sulphide bond of HFUS1 assessed by mass spectroscopy. F4, F5, F6 and F9 correspond to Formulations 4, 5, 6 and 9 respectively, as described in Table 4.

FIG. 4C is a bar chart showing Methionine 271 (M271) oxidation at chain B of relaxin of HFUS1 assessed by mass spectroscopy. F4, F5, F6 and F9 correspond to Formulations 4, 5, 6 and 9 respectively, as described in Table 4.

FIG. 5 illustrates the purity profile of HFUS1 in histidine-arginine HCl at pH 5.5 to 7.0, indicating the monthly change rates (%) of monomer, aggregation and fragmentation during storage at 40° C. (A), 25° C. (B) and 5° C. (C.).

FIG. 6 is a bar chart showing amino acid clipping at the relaxin C-terminus at pH ranging from 5.5 to 7.0, at 0° C., 5° C., 25° C. and 40° C.

FIG. 7 shows the purity loss of AZ3427 with protease inhibitor (PI) assessed by HPSEC.

FIG. 8 is a bar chart showing the amino acid clipping of AZ3427 with protease inhibitor (PI), assessed by mass spectroscopy.

FIG. 9 illustrates particle formation after storage at 5° C. (A) shows pH screening samples for a HFUS1 formulation in a histidine-arginine HCl at the 6-month storage timepoint (B) shows pH screening samples for a HFUS1 formulation in a histidine-arginine HCl at the 12-month storage timepoint (C) shows AZ3427 formulation optimization samples after 12 months of storage (various buffer, excipient and pH)—Formulations 1 to 8 represent Formulations 1 to 8 respectively as described in Table 4.

FIG. 10 demonstrates the FTIR spectrums of HFUS1 particles in comparison to protein and PS80 references. The highlighted boxes indicate IR signatures similar to protein reference and also traces of signatures of PS80.

FIG. 11 shows visual inspection of HFUS1 formulations in 2R vials after storage at 5° C. for (A) 9 months and (B) 12 months.

FIG. 12 shows visual inspection of HFUS1 formulations in 1 mL pre-filled syringes after storage at 5° C. for (A) 9 months and (B) 12 months.

FIG. 13 shows the particle counts/mL, measured by microflow imaging of HFUS1 formulations for (A) particles equal to or greater than 1 μm but less than 2 μm diameter; (B) particles equal to or greater than 2 μm diameter; and (C) particles equal to or greater than 10 μm diameter.

FIG. 14 is a total ion LC-MS chromatogram of PS-80 species in stressed and unstressed samples (POE is polyoxymethylene).

FIG. 15 shows the HPSEC of HFUS1 formulations with PS80 and P188 stored at (A) 40° C. for 3 months and (B) 5° C. for 12 months (PS80 samples) or 18 months (P188 samples). MPP is major product peak which is the sum of monomer and shoulder.

FIG. 16 shows the change in isoelectric point, measured by capillary isoelectric focusing (cIEF), of HFUS1 formulations with PS80 and P188 stored at 5° C. for 12 months (PS80 samples) and 18 months (P188 samples).

FIG. 17 shows the HPSEC of HFUS1 formulations stored at (A) 40°, (B) 40° C., (C) 25° C. and (D) 5° C. The percentage of rate change per month is indicated. “50L DEV LOT (P1) 0.04% P188 TARGET” corresponds to formulation F1 (target) in Table 6. The x-axis indicates changes in the tested formulation relative to F1 (target) and correlates with the formulations listed in Table 6.

FIG. 18 shows the capillary gel electrophoresis (CGE) of HFUS1 formulations at 0 month and 1 month after storage at (A) 40° C., (B) 25° C. and (C) 5° C. “50L DEV LOT (P1) 0.04% P188 TARGET” corresponds to formulation F1 (target) in Table 6. The x-axis indicates changes in the tested formulation relative to F1 (target) and correlates with the formulations listed in Table 6.

FIG. 19 shows the capillary isoelectric focusing (CIEF) of HFUS1 formulations stored at (A) 40° C., (B) 25° C. and (C) 5° C. The percentage of rate change per month is indicated. “50L DEV LOT (P1) 0.04% P188 TARGET” corresponds to formulation F1 (target) in Table 6. The x-axis indicates changes in the tested formulation relative to F1 (target) and correlates with the formulations listed in Table 6.

FIG. 20 shows the Micro-Flow imaging (MFI) of HFUS1 formulations stored for 3 months at (A) 40° C. for particles equal to or greater than 2 μm diameter, (B) 40° C. for particles equal to or greater than 10 μm diameter, (C) 5° C. for particles equal to or greater than 2 μm diameter and (D) 5° C. for particles equal to or greater than 10 μm diameter. “F0” to “F8” correspond to the formulations listed in Table 6.

FIG. 21 shows the HPSEC (percentage of monomers) of HFUS1 low concentration formulations stored at (A) 40° C., (B) 25° C. and (C) 5° C. “TARGET (0.25 MG/ML)”, “WORST CASE (0.25 MG/ML)”, “TARGET (1 MG/ML)” and “WORST CASE (1 MG/ML)” correspond to formulations P1 to P4 respectively in Table 7.

FIG. 22 shows the capillary gel electrophoresis (CGE) of HFUS1 low concentration formulations stored at (A) 40° C., (B) 25° C. and (C) 5° C. P1 to P4 correspond to formulations P1 to P4 respectively in Table 7.

FIG. 23 shows the capillary isoelectric focusing (CIEF) (main peak %) of HFUS1 low concentration formulations stored at (A) 40° C., (B) 25° C. and (C) 5° C. The percentage of rate change per month is indicated. P1 to P4 correspond to formulations P1 to P4 respectively in Table 7.

FIG. 24 shows micro-flow imaging (MFI) of HFUS1 low concentration formulations stored at 5° C., 25° C. and 40° C. (A) for particles equal to or greater than 25 μm diameter, and (B) for particles equal to or greater than 10 μm diameter. “TARGET (0.25 MG/ML)”, “WORST CASE (0.25 MG/ML)”, “TARGET (1 MG/ML)” and “WORST CASE (1 MG/ML)” correspond to formulations P1 to P4 respectively in Table 7.

FIG. 25 shows exemplary formats of the heterodimeric fusions according to some embodiments of the disclosure. The format of each fusion polypeptide of the heterodimeric fusion is given in terms of FcX, FcY, A, B, con and L, wherein FcX (“Fc Knob”) and FcY (“Fc Hole”) are two Fc regions comprising heterodimerisation-promoting amino acid mutations and/or modifications; A (“Rlx A”) and B (“Rlx B”) are Relaxin A chain and Relaxin B chain polypeptides; “con” is a connector polypeptide; L is a linker polypeptide, HC X and HC Y—heavy chains of an antibody, LC—light chain of an antibody, hinge—the hinge region of an antibody and Fab is Fab fragment of an antibody.

FIG. 26 shows particle count via Micro-Flow imaging (MFI) of various HFUS1 formulations stored at (A) 5° C. for particles equal to or greater than 2 μm diameter, (B) 5° C. for particles equal to or greater than 10 μm diameter, (C) 5° C. for particles equal to or greater than 25 μm diameter (D) 25° C. for particles equal to or greater than 2 μm diameter, (E) 25° C. for particles equal to or greater than 10 μm diameter, (F) 25° C. for particles equal to or greater than 25 μm diameter, (G) 40° C. for particles equal to or greater than 2 μm diameter, (H) 40° C. for particles equal to or greater than 10 μm diameter, and (I) 40° C. for particles equal to or greater than 25 μm diameter. Data corresponds to the formulations listed in Table 8 with “33 mg/ml”=PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

FIG. 27 shows particle count via light obscuration method (HIAC) of HFUS1 formulations stored at (A) 5° C. for particles equal to or greater than 2 μm diameter, (B) 5° C. for particles equal to or greater than 10 μm diameter, (C) 5° C. for particles equal to or greater than 25 μm diameter (D) 25° C. for particles equal to or greater than 2 μm diameter, (E) 25° C. for particles equal to or greater than 10 μm diameter, (F) 25° C. for particles equal to or greater than 25 μm diameter, (G) 40° C. for particles equal to or greater than 2 μm diameter, (H) 40° C. for particles equal to or greater than 10 μm diameter, and (I) 40° C. for particles equal to or greater than 25 μm diameter. Data corresponds to the formulations listed in Table 8 with “33 mg/ml”=PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

FIG. 28 shows the HPSEC of HFUS1 formulations stored at (A) 5° C., (B) 25° C., and (C) 40° C. The percentage of monomer rate change per month is indicated. Data correlates with the formulations listed in Table 8 with “33 mg/ml”=PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

FIG. 29 shows the capillary isoelectric focusing (cIEF) of HFUS1 formulations stored at (A) 5° C., (B) 25° C. and (C) 40° C. Data correlates with the formulations listed in Table 8 with “33 mg/ml” =PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

FIG. 30 shows the potency of HFUS1 formulations stored at (A) 5° C., (B) 25° C. and (C) 40° C. Data correlates with the formulations listed in Table 8 with “33 mg/ml”=PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

FIG. 31 shows clipping of amino acid at the C-terminus of chain B of Relaxin of HFUS1 formulations assessed by RP-HPLC stored at (A) 5° C., (B) 25° C. and (C) 40° C. Data correlates with the formulations listed in Table 8 with “33 mg/ml”=PFS—Control; “33 mg/mL pH 6”=PFS—1; “33 mg/mL pH 7”=PFS—2; “33 mg/mL low Arg HCl”=PFS—3; “33 mg/mL high Arg HCl”=PFS—4; “33 mg/mL 0.02% P188”=PFS—5; “33 mg/mL 0.06% P188”=PFS—6.

### DETAILED DESCRIPTION OF THE DISCLOSURE

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of contemplated embodiments described herein.

References to subject-matter disclosed or described “herein” relates to subject-matter disclosed or described anywhere in the present application.

### Overview

During the formulation development of the heterodimeric fusions, e.g. HFUS1, some challenges pertaining to the shelf-life and stability of the molecule were met. These included: a tendency of HFUS1 to self-associate, leading to aggregation; a tendency for amino acid clipping at certain pH ranges and a high susceptibility to form particles.

Thus, the present disclosure describes some purposely designed studies to identify the root cause of instabilities with HFUS1, and formulation development and optimisation work to identify a stable liquid formulation for the molecule to meet its drug product shelf-life requirement.

The present inventors have demonstrated that HFUS1 tends to self-associate (see Example 1). High levels of self-association of molecules can lead to the formation of soluble aggregates which can become precursors of insoluble, large-size aggregates, and particles eventually, significantly impacting the stability profile of the molecule. The present inventors established that formulation optimisation was required to reduce this self-association and aggregation tendency. Surprisingly, the surfactant in the formulation was determined to play a key role in particle formation associated with free fatty acids (FFA) which can serve as a nucleus to trigger HFUS1 protein aggregation. Optimising the surfactant significantly reduced particle formation and reduced the tendency for aggregation (see Example 3). The optimisation of the pH, buffer and excipient used in the formulation of HFUS1 were also found to further reduce aggregation. Overall, the histidine-arginine HCl system (i.e. histidine-based buffer system with arginine hydrochloride ionic excipient) was identified to provide the highest colloidal and conformational stability to HFUS1 (see Example 2).

The present inventors have also established that proteases are likely the cause of the fragmentation and clipping of HFUS1. This enzymatic activity is more likely to happen at lower pH where the proteases are most effective in cutting down the molecule. Therefore, it is important that the pH of the formulation is maintained at a higher range as demonstrated in the pH optimisation study (see Example 2). An optimal pH range of 5.5 to 7.5, in particular of 6 to 7, more particularly of 6.5, minimises the chemical degradation impact such as fragmentation and amino acid (AA) clipping of the molecule.

As mentioned above, the present inventors identified the formation of visible particles in the HFUS1 formulations over time (see Example 3). Several root causes for the formation of the particles were hypothesized as explained in Example 3. Surprisingly, the inventors established that the presence of the surfactant PS80 was the cause of the particle formation. The inventors established that enzymatic hydrolysis of the ester bond of PS80 by lipoprotein lipase (LPL) present in the formulation was likely the cause of the degradation of PS80, which in turn resulted in the formation of impurities such as free fatty acids (FFA) which can serve as nucleus to trigger HFUS1 protein aggregation leading to particles. In the meantime, due to degradation, the PS80 level decreased, therefore losing its protection/surfactant effect to prevent particle formation during storage. The use of lipase-resistant surfactant, such as poloxamer 188 (P188) and D-a-Tocopherol polyethylene glycol succinate (TPGS), was able to mitigate the particle formation in the HFUS1 formulations.

The impact of HFUS1 concentration on stability was also evaluated and HFUS1 showed good stability in formulations described herein across a broad range of HFUS1 concentrations, with data for HFUS1 concentrations from 0.25 mg/mL to 50 mg/mL, in particular for formulations with HFUS1 concentrations of 0.25 mg/mL, 1 mg/mL, 5 mg/mL, 33 mg/mL, and 50 mg/mL (see Examples 4, 5 and 6).

In summary, a histidine-Arginine HCl system was selected for its effectiveness in reducing the self-association tendency of the molecule and an optimised pH range was identified which prevented AA clipping and fragmentation of the molecule effectively (see Example 2). Further, detailed investigational work was conducted to understand the cause of the particle formation issue and PS80 was identified as the root cause. This led to the optimisation of the surfactant in the formulation system and alternative surfactants such as P188 and TPGS mitigated the particle formation issue (see Example 3). P188 was selected as the lead surfactant for the formulation. Finally, a comprehensive formulation stability study was conducted to evaluate the robustness of the formulation system. The histidine/histidine hydrochloride-arginine HCl formulation showed excellent stability profile and robustness. The impact of HFUS1 concentration on stability was also evaluated and HFUS1 also showed good stability across the broad range of concentrations tested (see Examples 4 to 6).

Therefore, provided herein is a pharmaceutical formulation comprising a heterodimeric fusion, e.g. HFUS1, and a lipase-resistant surfactant, wherein the heterodimeric fusion comprises:
(i) a first heterodimerisation domain connected to at least one Relaxin A chain polypeptide or a variant thereof; and
(ii) a second heterodimerisation domain connected to at least one Relaxin B chain polypeptide or a variant thereof,
wherein the first heterodimerisation domain heterodimerises with the second heterodimerisation domain, and wherein the heterodimeric fusion has Relaxin activity.

Also provided herein is a pharmaceutical formulation as described herein for use in therapy. Also provided is a method of treating a subject with a disease or disorder, the method comprising administering the pharmaceutical formulation as described herein to the subject.

Also provided herein is a pharmaceutical formulation as described herein for use in the treatment of a subject with heart failure, optionally heart failure with pulmonary hypertension (e.g. Group 2 Pulmonary Hypertension). Also provided herein is a method of treating a subject with heart failure, optionally heart failure with pulmonary hypertension (e.g. Group 2 Pulmonary Hypertension), the method comprising administering the pharmaceutical formulation as described herein to the subject.

Also provided herein is a kit comprising the pharmaceutical formulation as described herein.

### Heterodimeric Fusion Having Relaxin Activity

### Relaxin

The pharmaceutical formulation of the disclosure comprises a heterodimeric fusion having Relaxin activity, e.g. HFUS1.

As described in WO2021/255127, heterodimeric fusions described herein, e.g. HFUS1, may exhibit Relaxin activity when the Relaxin A chain and the Relaxin B chain are not covalently linked to each other through an amino acid linker. Advantageously, heterodimerisation of the heterodimerisation domains induces correct folding and heterodimerisation of the Relaxin A and Relaxin B chains (see Example 2 of WO2021/255127). In addition, unlike wild-type Relaxin proteins, the heterodimeric fusions, e.g. HFUS1 do not require endoproteolytic processing for biological activity.

As used herein, the term “heterodimeric fusion” refers to a heterodimer of fusion polypeptides, wherein one fusion polypeptide comprises a first heterodimerisation domain connected to a first subunit of a heterodimeric protein (e.g. Relaxin A chain), and the other fusion polypeptide comprises a second heterodimerisation domain connected to a second subunit of a heterodimeric protein (e.g. Relaxin B chain). In some embodiments, the heterodimeric fusion is HFUS1 (also termed RELAX0023). HFUS1 is a heterodimeric fusion consisting of a fusion polypeptide with the amino acid sequence of SEQ ID NO: 11 and a fusion polypeptide with the amino acid sequence of SEQ ID NO: 20.

The heterodimeric fusions used in the formulation of the present disclosure may comprise Relaxin A and B chain polypeptides from the group of Relaxins selected from Relaxin-1, Relaxin-2 and Relaxin-3. In particular embodiments, the Relaxin A chain polypeptide is a Relaxin-2 A chain polypeptide or a variant thereof; and the Relaxin B chain polypeptide is a Relaxin-2 B chain polypeptide or a variant thereof. In particular embodiments, the Relaxin A chain polypeptide comprises a human Relaxin-2 A chain polypeptide or a variant thereof and a human Relaxin-2 B chain polypeptide or a variant thereof.

The terms “chain”, “polypeptide” and “peptide” may be used interchangeably herein to refer to a chain of two or more amino acids linked through peptide bonds.

In some embodiments, the Relaxin-2 A chain polypeptide has the sequence as set forth in SEQ ID NO: 1 or a variant thereof and the Relaxin-2 B chain polypeptide has the sequence as set forth in SEQ ID NO: 2 or a variant thereof. Variants may comprise one or more amino acid substitutions, deletions and/or insertions. In some embodiments, the Relaxin-2 A chain polypeptide comprises one or more amino acid mutations selected from K9E, K9H, K9L, K9M, R18E, R18H, R22A, R22I, R22M, R22Q, R22S, R22Y, F23E, F23A and F231. In a particular embodiment Relaxin-2 A chain comprises the amino acid mutation K9H.

Relaxin A and B chain variants are known in the art. In addition, guidance on the design of Relaxin A and B chain variants is available to the skilled person. For example, it will be understood that variants may retain those amino acids that are required for Relaxin function. For example, Relaxin-2 B chain variants may comprise the conserved motif Arg-X-X-X-Arg-X-X-Ile (Claasz A A et al. (2002) Eur. J. Biochem. 269 (24): 6287-6293) or Arg-X-X-X-Arg-X-X-Val (Bathgate R A et al. (2013) Physiol Rev. 93 (1): 405-480). Variants may comprise one or more amino acid substitutions and/or insertions. For example, Relaxin-2 B chain variants may have one or more additional amino acids for example K30 and R31 and N-terminal V-2, A-1 and M-1 compared to SEQ ID NO: 62. Alternatively or in addition, variants may comprise one or more amino acid derivatives. For example, the first amino acid of Relaxin-2 B chain variants may be pyroglutamate.

In particular embodiments, the Relaxin A chain and the Relaxin B chain are covalently bound by two inter-chain disulphide bonds (see Example 2 of WO2021/255127).

The Relaxin family of peptides mediate their biological effects, at least in part, through the activation of G protein-coupled receptors (GPCRs), and the subsequent stimulation or inhibition of the CAMP signalling pathway by the Gs or Gi protein subunit, respectively. Relaxin-2 is known to activate the GPCR RXFP1 (also known as LGR7) and, to a lesser degree, the GPCR RXFP2 (also known as LGR8), thus stimulating the Gs-CAMP-dependent signalling pathway, leading to an increase in the second messenger molecule cAMP.

As used herein, the term “Relaxin activity” refers to the ability of a Relaxin molecule to bind to a Relaxin receptor, and/or activate said Relaxin receptor and/or initiate a signalling cascade inside the cell. In embodiments in which the Relaxin activity is Relaxin-2 activity, Relaxin activity may refer to the ability to bind and/or activate the receptor RXFP1 and/or RXFP2. The term “Relaxin activity” may be used interchangeably with “biological activity”.

Relaxin activity may be determined by measuring binding of a Relaxin molecule to a Relaxin receptor, and/or by measuring downstream events from binding to a Relaxin receptor.

Relaxin activity may be determined in vitro and/or in vivo. In some embodiments, Relaxin activity is determined in vitro.

Relaxin activity may be determined by measuring the amount and/or presence of a molecule downstream from Relaxin activation of a receptor. For example, Relaxin activity may be determined by measuring cAMP production following Relaxin activation of a receptor. Methods for the detection of Relaxin-induced CAMP generation are known in the art. Such methods include CAMP ELISA, HTRF CAMP assays and the HitHunter®CAMP assay. In some embodiments, Relaxin activity is determined by measuring Relaxin-induced CAMP production by HTRF CAMP assay, e.g. as performed in Example 3 of WO2021/255127. Relaxin activity may also be determined by measuring nitric oxide (NO) production following Relaxin activation of a receptor. Relaxin activity may also be determined by measuring the activation of a molecule downstream from Relaxin activation of a receptor. For example, Relaxin activity may be determined by measuring activation of p42/44 MAPK.

Alternatively or in addition, Relaxin activity may be determined by measuring the activation of a known Relaxin target gene. For example, Relaxin activity may be determined by measuring the activation of the transcription of the known Relaxin target gene, VEGF, in THP-1 cells. Methods to determine activation of transcription of a gene are known in the art and include quantitative PCR analysis of the mRNA. The relative expression of VEGF mRNA can be measured by quantitative real-time PCR induction of VEGF transcripts following incubation of THP-1 cells with Relaxin as described in Xiao et al. (2013) Nat Commun. 4:1953.

Alternatively or in addition, Relaxin activity may be determined by measuring one or more downstream effects of Relaxin. For example, reduction of cardiac hypertrophy can be measured by echocardiography, left ventricular weight relative to body weight and/or tibia length according to standard methods. In another example, Relaxin activity may be determined by measuring fibrosis reduction by Masson's Trichrome stain. In another example, Relax in activity may be determined by measuring modulation of connective tissue metabolism, such as the inhibition of profibrotic factors (such as TGF-beta), inhibition of fibroblast proliferation and differentiation, and/or activation of MMP-mediated extracellular matrix degradation (Bathgate R A et al. (2013) Physiol Rev. 93 (1): 405-480).

In some embodiments, Relaxin activity is determined by measuring reversal of isoproterenol-induced cardiac hypertrophy (measured as heart weight relative to tibial length) and fibrosis (measured as collagen content relative to heart weight), e.g. as performed in Example 7 of WO2021/255127.

The activity of the heterodimeric fusions of the disclosure, e.g. HFUS1, may be determined in relation to a reference Relaxin protein. In some embodiments, the reference Relaxin protein is a recombinant protein. In particular embodiments, the reference Relaxin protein is a Relaxin protein having the Relaxin A chain and Relaxin B chain array of a mature Relaxin protein. Recombinant Relaxins having the Relaxin A chain and Relaxin B chain array of a mature Relaxin protein are commercially available. For example, recombinant human Relaxin-2, murine Relaxin-1 and INSL3 are available from R&D systems (catalogue numbers 6586-RN, 6637-RN and 4544-NS, respectively).

In some embodiments, the reference Relaxin protein has the same Relaxin A and B chains as the heterodimeric fusion of the disclosure or differs from the Relaxin A and B chains of the heterodimeric fusion of the disclosure by up to 10 amino acids, for example 1 or 2 amino acids. In some embodiments, the first amino acid of the B chain of the reference Relaxin-2 is D and this amino acid is deleted in the Relaxin B chain of the heterodimeric fusion of the disclosure.

The reference Relaxin protein may be selected from:
(i) recombinant human Relaxin-2 (referred to herein as RELAX0013); and
(ii) recombinant murine Relaxin-1 (referred to herein as RELAX0014); and
(iii) recombinant Fc-fused Relaxin-2 in which the Relaxin A and Relaxin B are fused in a single chain, and wherein Fc is a half-life extending Fc region (referred to herein as RELAX0010 and described in WO2018/138170); and
(iv) recombinant Fc-fused Relaxin-2 in which the Relaxin A and Relaxin B are fused in a single chain, and wherein Fc is a half-life extending Fc region (referred to herein as RELAX0009 and described in WO2018/138170); and
(v) recombinant Fc-fused Relaxin-2 in which the Relaxin A and Relaxin B are fused in a single chain (referred to herein as RELAX0126 and described in WO 2013/004607); and
(vi) recombinant Fc-fused Relaxin-2 in which the Relaxin A and Relaxin B are fused in a single chain (referred to herein as RELAX0127 and described in WO 2013/004607); and
(vii) recombinant Fc-fused Relaxin in which the Relaxin A and Relaxin B are fused in a single chain (referred to herein as RELAX0128 and described in WO 2013/004607).

In particular embodiments, the reference Relaxin protein is a Relaxin-2 protein having the Relaxin-2 chain A and Relaxin-2 B chain array of a mature Relaxin-2 protein as disclosed under UniProtKB/Swiss-Prot Accession Number P04090.1.

The heterodimeric fusions of the disclosure, e.g. HFUS1, may be considered to have Relaxin activity if they show at least a proportion of the activity of a reference Relaxin protein. For example, a fusion polypeptide may be considered to have Relaxin activity if it has at least about half of the activity of a reference Relaxin protein. A heterodimeric fusion of the disclosure may be considered to have Relaxin activity if the ratio of the activity of said fusion polypeptide over the activity of a reference Relaxin protein is between about 10⁻⁵ and about 1, between about 10⁻⁴ and about 1, between about 10⁻³ and about 1, between about 10⁻² and about 1, between about 1/50 and about 1, between about 1/20 and about 1, between about 1/15 and about 1, between about 1/10 and about 1, between about ⅕ and about 1, or between about ½ and about 1. Alternatively, a heterodimeric fusion of the disclosure may be considered to have Relaxin activity if the ratio of the activity of said fusion polypeptide over the activity of a reference Relaxin protein is between about 1 and about 10⁵, between about 1 and about 10⁴, between about 1 and about 10³, between about 1 about 100, between about 1 and about 50, between about 1 and about 20, between about 1 and about 15, between about 1 and about 10, between about 1 and about 5, or between about 1 and about 2.

In some embodiments, the Relaxin activity of the heterodimeric fusion, e.g. HFUS1, over the Relaxin activity of a reference Relaxin protein is between about 0.001 and about 10.

Relaxin activity may be determined as an EC50 value. As used herein the term “EC50” (half maximal effective concentration) refers to the effective concentration of a therapeutic compound which induces a response halfway between the baseline and maximum after a specified exposure time.

### Heterodimerisation Domains

The heterodimeric fusions, e.g. HFUS1, used in the formulation of the disclosure comprise a first heterodimerisation domain and a second heterodimerisation domain. In particular embodiments, the first and second heterodimerisation domains are derived from an immunoglobulin Fc region.

The term “Fc region” defines the C-terminal region of an immunoglobulin heavy chain, which may be generated by papain digestion of an intact antibody. The Fc region of an immunoglobulin generally comprises two constant domains, a CH2 domain and a CH3 domain, and optionally comprises a CH4 domain.

The first and second Fc regions may comprise the immunoglobulin domains CH2 and/or CH3. In particular embodiments, the first and second Fc regions comprise the immunoglobulin domains CH2 and CH3.

The Fc region may be derived from an immunoglobulin (e.g. IgG) from any species, particularly human (e.g. human IgG). In embodiments in which the Fc region is derived from IgG, the Fc region may be derived from an IgG of any subclass (e.g. IgG1, IgG2, IgG3, IgG4), particularly IgG1. In particular embodiments, the first and second Fc regions are derived from a human IgG1 immunoglobulin. In other embodiments, the first and second Fc regions are derived from a human IgG4 immunoglobulin.

In particular embodiments, the first and second Fc regions comprise heterodimerisation-promoting amino acid mutations and/or modifications. Such modifications may include the introduction of asymmetric complementary modifications into each of the first and second Fc regions, such that both chains are compatible with each other and thus able to form a heterodimer, but each chain is not able to dimerize with itself. Such modifications may encompass insertions, deletions, conservative and non-conservative substitutions and rearrangements. Incorporating such modifications provides a method for increasing the yield of heterodimers produced by recombinant cell culture over other unwanted end-products such as homodimers.

The first and second Fc regions may comprise any heterodimerisation-promoting amino acid mutations and/or modifications known in the art. A combination of modifications may be used to maximise the efficiency of assembly while minimising the impact on antibody stability.

In the “knob in hole” method, heterodimerisation may be promoted by the introduction of steric hindrance between contacting residues. A “protrusion” is generated by replacing one or more small amino acid side chains from the interface of one Fc region (“Fc Knob”) with larger side chains (e.g. tyrosine or tryptophan). Compensatory “cavities” of identical or similar size to the large side chain(s) are created on the interface of the other Fc region (“Fc Hole”) by replacing amino acid having large side chains with amino acids having smaller ones (e.g. alanine or valine). “Knob-in-holes” modifications are described in detail e.g. Ridgway J B et al. (1996) Protein Eng. 9 (7) 617-621; Merchant A M et al. (1998) Nat. Biotechnol. 16 (7): 677-681.

Other modifications which may be used to generate heterodimers include but are not limited to those which create favourable electrostatic interactions between the two Fc regions. For example, one or more positively charged amino acids may be introduced into one Fc region, and one or more negatively charged amino acids may be introduced into a corresponding position in the other Fc region. Alternatively or in addition, the Fc regions may be modified to include mutations that introduce cysteine residues capable of forming a disulphide bond. Alternatively or in addition, the Fc regions may comprise one or more modification(s) to the hydrophilic and hydrophobic residues at the interface between chains, in order make heterodimer formation more entropically and enthalpically favourable than homodimer formation.

Thus, in some embodiments, the heterodimerisation-promoting amino acid mutations and/or modifications create steric hindrance between contacting residues (e.g. by “knob-in-hole”), create favourable electrostatic interactions between the two Fc regions, introduce cysteine residues capable of forming a disulphide bond and/or modify the hydrophilic and hydrophobic residues at the interface between the two Fc regions.

In particular embodiments, the heterodimerisation-promoting amino acid mutations are “Fc Knob” and “Fc Hole” mutations. In particular embodiments, the “Fc Knob” and “Fc Hole” mutations are present in the CH3 domains.

In some embodiments, the first and second Fc regions are derived from a human IgG1 immunoglobulin and comprise “Fc X” and “Fc Y” with mutations in the CH3 domains, wherein the “Fc X” and “Fc Y” mutations are selected from the combinations set forth in Table 1 (or conservative substitutions thereof).

TABLE 1
“Fc X” and “Fc Y” mutations

| Combination | | |
|---|---|---|
| No. | Fc X mutation(s)* | Fc Y mutation(s)* |
| 1 | D399C | K392C |
| 2 | D399S | K392S |
| 3 | Y349C | S354C |
| 4 | Y349C | E356C |
| 5 | Y349C | E357C |
| 6 | L351C | S354C |
| 7 | T394C | V397C |
| 8 | T366W | T366S:L368A:Y407V |
| 9 | T366W:D399C | T366S:L368A:K392C:Y407V |
| 10 | T366W:K392C | T366S:0099C:L368A:Y407V |
| 11 | S354C:T366W | Y349C:T366S:L368A:Y407V |
| 12 | Y349C:T366W | S354C:T366S:L368A:Y407V |
| 13 | E356C:T366W | Y349C:T366S:L368A:Y407V |
| 14 | Y349C:T366W | E356C:T366S:L368A:Y41J7V |
| 15 | E357C:T366W | Y349C:T366S:L368A:Y407V |
| 16 | Y349C:T366W | E357C:T366S:L368A:Y407V |
| 17 | S364H/F405A | Y349T/T394F |
| 18 | T350V/L351Y/F405A/Y407V | T350V/T366L/K392L/T394W |
| 19 | K360D/D399M/Y407A | E345R/Q347R/T366V/K409V |
| 20 | K409D/K392D | D399K/E356K |
| 21 | K360E/K409W | Q347R/D399V/F405T |
| 22 | K360E/K409W/Y349C | Q347R/D399V/F405T/S354C |
| 23 | K370E/K409W | E357N/D399V/F405T |
| 24 | T366Y | Y407T |

*wherein the amino acid numbering is according to the EU index as in Kabat.

In particular embodiments the “Fc Y” is the “Fc Hole” with mutations Y349C, T366S, L368A and Y407V, or conservative substitutions thereof, and the “Fc X” is the “Fc Knob” with mutations S354C and T366W, or conservative substitutions thereof, wherein the amino acid numbering is according to the EU index as in Kabat.

The term “EU index as in Kabat” refers to the numbering system of the human IgG1 EU antibody described in Kabat E A et al. (1991) Sequences of Proteins of Immunological Interest, 5th ed. Public Health Service. National Institutes of Health. Bethesda, MD. All amino acid positions referenced in the present application refer to EU index positions.

In some embodiments, the first Fc region has “Fc Hole” mutations, and the second Fc region has “Fc Knob” mutations. In alternative embodiments, the first Fc region has “Fc Knob” mutations, and the second Fc region has “Fc Hole” mutations.

It will be understood that the Fc regions may further comprise other amino acid modifications relative to a wild-type Fc region. The Fc region may be modified to e.g. increase the affinity of the IgG molecule for the FcRn. WO 02/060919 discloses modified immunoglobulins comprising an Fc region having one or more amino acid modifications and is incorporated herein in its entirety by reference. Methods of making Fc regions with one or more amino acid modifications are known in the art.

In some embodiments, the first and/or second Fc region may comprise one or more amino acid modifications to reduce or abolish the effector function of the Fc region. In some embodiments, the amino acid modifications reduce or circumvent cytotoxicity, for example antibody-dependent cell-mediated cytotoxicity (ADCC) and complement-dependent cytotoxicity (CDC).

In some embodiments, the first and/or second Fc region may comprise one or more amino acid modifications to increase the half-life of the heterodimeric fusion, e.g. HFUS1.

In some embodiments, the first and/or second Fc region comprises at least one of the following combinations of amino acid mutations:
(i) M252Y, S254T and T256E, or conservative substitutions thereof;
(ii) L234F, L235Q and K322Q, or conservative substitutions thereof;
(iii) L234F, L235E and P331S, or conservative substitutions thereof;
(iv) M252Y, S254T, T256E, L234F, L235Q and K322Q, or conservative substitutions thereof; or
(v) M252Y, S254T, T256E, L234F, L235E and P331S, or conservative substitutions thereof,
wherein the amino acid numbering is according to the EU index as in Kabat.

In some embodiments, the first and/or second Fc region may comprise the amino acid mutations L234F, L235E and P331S, or conservative substitutions thereof, wherein the amino acid numbering is according to the EU index as in Kabat.

In some embodiments, the Fc region comprising “Fc Hole” mutations has the sequence set forth in SEQ ID NO: 3 or variants thereof, and the Fc region comprising “Fc Knob” mutations has the sequence set forth in SEQ ID NO:4 or variants thereof.

In some embodiments, the Fc regions comprise a SEQ ID NO: 3 variant having the amino acid mutation Y349C reverted to Y349 and a SEQ ID NO: 4 variant having the amino acid mutation S354C reverted to S354, such that the Fc regions are unable to form a stabilising disulphide bond.

In some embodiments, the Fc regions comprise a SEQ ID NO: 3 variant and/or SEQ ID NO: 4 variant, wherein the first five residues DKTHTCPPC (SEQ ID NO: 69) are modified. In some embodiments, this region is replaced with the sequence DKTHTACPPC (SEQ ID NO: 70). In alternative embodiments, this region is replaced with the sequence GGAGGACPPC (SEQ ID NO: 71). In alternative embodiments, this region is replaced with the sequence ACPPC (SEQ ID NO: 72).

In alternative embodiments, the first and second heterodimerisation domains are derived from an immunoglobulin Fab region. In some embodiments, the heterodimerisation domains comprise CH1 and CL regions. It has been found that Fab regions comprising L and Fd chains mediate efficient heterodimerisation (Schoonjans R et al. (2000) J. Immunol. 165 (12): 7050-7057). Thus, in alternative embodiments, the heterodimerisation domains comprise L and Fd chains. In some embodiments, the L and Fd chains heterodimerise to form a disulphide-bridge stabilised heterodimer.

In yet further alternative embodiments, the first and second heterodimerisation domains heterodimerise to form parallel coiled coils. Heterodimeric coiled coils are described e.g. in Aronsson et al. (2015) Sci. Rep. 5:14063. In some embodiments, the heterodimerisation domains comprise amino acid mutations and/or modifications to prevent formation of undesired folded assemblies and/or to promote formation of parallel coiled coils.

The first and second heterodimerisation domains (e.g. first and second Fc regions) may form a half-life extending moiety. Thus, in some embodiments the heterodimeric fusions of the disclosure, e.g. HFUS1, have an extended half-life compared to a reference Relaxin.

As used herein, the term “half-life” is used to refer to the time taken for the concentration of fusion protein in plasma to decline to 50% of its original level. The “half-life” of a protein in plasma may depend on different factors such as the size of the protein, its stability, its clearance rate, turnover rate, in vivo proteolytic degradation, the rate of absorption by the body or specific tissues, etc. Methods to determine the half-life of proteins are known in the art and are described in the Examples below.

As shown in WO2021/255127, heterodimeric fusions as described herein, e.g. HFUS1, having first and second heterodimerisation domains derived from an immunoglobulin Fc have a half-life of at least 5 hours in mouse models (see Example 6 of WO2021/255127). In comparison, the half-life of human Relaxin-2 following IV administration is about 0.09+/−0.04 hours, i.e. 5.4+/−2.4 minutes in humans (Chen S A et al. (1993) Pharm. Res. 10 (6): 834-838).

It will be recognised that an extended half-life is advantageous, as it permits the therapeutic proteins to be administered according to a safe and convenient dosing schedule, e.g. lower doses that can be administered less frequently. Moreover, the achievement of lower doses may provide further advantages such as the provision of an improved safety profile and/or the activation of multiple mechanisms of action in vivo.

### Connectors

One or both of the Relaxin A and B chains may be connected to their respective heterodimerisation domains by a connector polypeptide. In some embodiments, the Relaxin A chain is connected to the first heterodimerisation domain (e.g. first Fc region) via a connector polypeptide, and the Relaxin B chain is connected to the second heterodimerisation domain (e.g. second Fc region) via a connector polypeptide.

The connector polypeptide may be any suitable length, for example between about 6 and 40 amino acids in length, such as between about 6 and 21 amino acids in length. In some embodiments, the connector polypeptide is at least 6 amino acid residues in length, particularly at least 11 amino acids in length, particularly at least 16 amino acids in length. In some embodiments, the connector polypeptide is less than 40 amino acids in length. Connector polypeptides of different or the same lengths can be used for each arm of the heterodimeric fusions as described herein, e.g. HFUS1. In some embodiments, at least one connector polypeptide has a length of 21 amino acids. In particular embodiments, both connector polypeptides have a length of 21 amino acids. The connector polypeptides can have any amino acid sequence. Connector polypeptides of different or the same amino acid formulations can be used for each arm of the heterodimeric fusions as described herein, e.g. HFUS1.

In some embodiments, one or both connector polypeptides comprise proline and alanine repeats (PA)x (SEQ ID NO: 73). In particular embodiments, x is of between 3 and 15, optionally wherein the connector polypeptide has a length greater than 16 amino acids, optionally wherein the connector polypeptide is composed of the 21 amino acid sequence PAPAPAPAPAPAPAPAPAPAG (SEQ ID NO: 6).

In some embodiments, one or both connector polypeptides comprise glycine and serine repeats such as those described in Chen X et al. (2013) Adv. Drug. Deliv. Rev. 65 (10): 1357-1369. In some embodiments, one or both connector polypeptides comprise the motif (GGGGS)n (SEQ ID NO: 74), wherein n may be between 1 and 8, for instance wherein n is 4. In some embodiments, one or more connector polypeptide is composed of the 21 amino acid sequence GGGGSGGGGSGGGGSGGGGGS (SEQ ID NO: 5). In certain embodiments, both connector polypeptides are composed of the 21 amino acid sequence GGGGSGGGGSGGGGSGGGGGS (SEQ ID NO: 5).

In some embodiments, one connector polypeptide comprises proline and alanine repeats as described herein, and the other connector polypeptide comprises glycine and serine repeats as described herein.

Alternatively, one or both of the Relaxin A and B chains may be connected to their respective heterodimerisation domains by a synthetic connector polypeptide, such as a polyethylene glycol (PEG) polymer chain. Thus, the Relaxin A chain may be connected to the first heterodimerisation domain (e.g. first Fc region) via a synthetic connector, such as a polyethylene glycol (PEG) polymer chain, and the Relaxin B chain may be connected to the second heterodimerisation domain (e.g. second Fc region) via a synthetic connector, such as a polyethylene glycol (PEG) polymer chain, wherein the synthetic connector may be covalently or non-covalently attached to the heterodimerisation domain (e.g. Fc region). PEGylation, that is the process of attaching PEG polymer chains to a molecule, can be carried out according to methods known in the art.

### Stability

As shown in WO2021/255127, heterodimeric fusions as described herein, e.g. HFUS1, have unexpected superior physical and chemical stability. Thus, in some embodiments the heterodimeric fusions as described herein, e.g. HFUS1, have superior physical and/or chemical stability compared to a reference Relaxin protein.

Physical stability of Relaxin may be determined by measuring purity and aggregation, for example by HP-SEC as in Example 9 of WO2021/255127. Chemical stability of Relaxin may be determined by measuring fragmentation and modification of the molecule, for example by LC-MS as in Example 9 of WO2021/255127.

Surprisingly, as shown in WO2021/255127 heterodimeric fusions as described herein, e.g. HFUS1, have superior physical and chemical stability compared to recombinant Fc-fused Relaxin in which the Relaxin A and Relaxin B are fused in a single chain (as opposed to Relaxin A and B in separate fusion polypeptides). WO 2013/004607 describes recombinant single chain Relaxin fusion polypeptides fused to an immunoglobulin Fc region, for example the fusion polypeptides referred to herein as RELAX0127 and RELAX0128. Thus, in some embodiments, the heterodimeric fusions as described herein, e.g. HFUS1, have superior physical and/or chemical stability compared to RELAX0127 and RELAX0128.

The heterodimeric fusion, e.g. HFUS1, may comprise a half-life extending moiety in addition to the first and second heterodimerisation domains. In some embodiments, the half-life extending moiety is a proteinaceous half-life extending moiety. The proteinaceous half-life extending moiety may be selected from the group consisting of an Fc region of an immunoglobulin, albumin-binding domain and serum albumin. In further embodiments, the half-life extending moiety is a chemical entity that is not a protein or peptide, such as a polyethylene glycol (PEG) polymer chain.

The half-life extending moiety may be attached at the N-terminus or the C-terminus of the first or second heterodimerisation domain. In some embodiments, the half-life extending moiety is attached at the N-terminus of the first or second heterodimerisation domain. In other embodiments, the half-life extending moiety is attached at the C-terminus of the first or second heterodimerisation domain. Methods for attaching the half-life extending moiety to the heterodimeric fusion, e.g. HFUS1, are known in the art. For example, the half-life extending moiety may be attached by chemical conjugation or recombinant technology. The half-life extending moiety may be attached to the heterodimeric fusion, e.g. HFUS1, directly or through a connector (e.g. connector polypeptide). The use of a connector polypeptide may be particularly appropriate when the fusion polypeptide comprises a proteinaceous half-life extending moiety such as an Fc region.

### Exemplary Embodiments

The heterodimeric fusions used in the formulation of the disclosure, e.g. HFUS1, may have a variety of formats and/or sequences.

The term “fusion polypeptide” and “fusion polypeptides” may be used to refer to the first heterodimerisation domain fused to a Relaxin A chain, and/or the second heterodimerisation domain fused to a Relaxin B chain. The fusion polypeptides used in the formulation of the disclosure may be recombinant fusion polypeptides, i.e. which have been created by recombinant DNA technology.

In particular embodiments, the C-terminus of the first heterodimerisation domain (e.g. first Fc region) is connected to the N-terminus of the Relaxin A chain and the C-terminus of the second heterodimerisation domain (e.g. second Fc region) is connected to the N-terminus of the Relaxin B chain. In some embodiments, the Relaxin A chain polypeptide and/or the Relaxin B chain polypeptide have a free C-terminus.

In alternative embodiments, the N-terminus of the first heterodimerisation domain (e.g. first Fc region) is connected to the C-terminus of the Relaxin A chain and the N-terminus of the second heterodimerisation domain (e.g. second Fc region) is connected to the C-terminus of the Relaxin B chain. In some embodiments, the Relaxin A chain polypeptide and/or the Relaxin B chain polypeptide have a free N-terminus.

The heterodimeric fusion used in the formulation of the disclosure, e.g. HFUS1, may further comprise one or more Fabs. In some embodiments, the heterodimeric fusion comprises one Fab linked to the N-terminus of the first heterodimerisation domain (e.g. first Fc region) and a second Fab linked to the N-terminus of the second heterodimerisation domain (e.g. second Fc region).

The heterodimeric fusion used in the formulation of the disclosure, e.g. HFUS1, may further comprise a second Relaxin A chain polypeptide or variant thereof and a second Relaxin B chain polypeptide or variant thereof. In some embodiments, the second Relaxin A chain polypeptide or variant thereof is connected to the N-terminus of the first heterodimerisation domain (e.g. first Fc region) and the second Relaxin B chain polypeptide or variant thereof is connected to the N-terminus of the second heterodimerisation domain (e.g. second Fc region), optionally wherein the second Relaxin A chain is connected to the first heterodimerisation domain (e.g. first Fc region) via a connector (e.g. connector polypeptide) and the second Relaxin B chain is connected to the second heterodimerisation domain (e.g. second Fc region) via a connector (e.g. connector polypeptide).

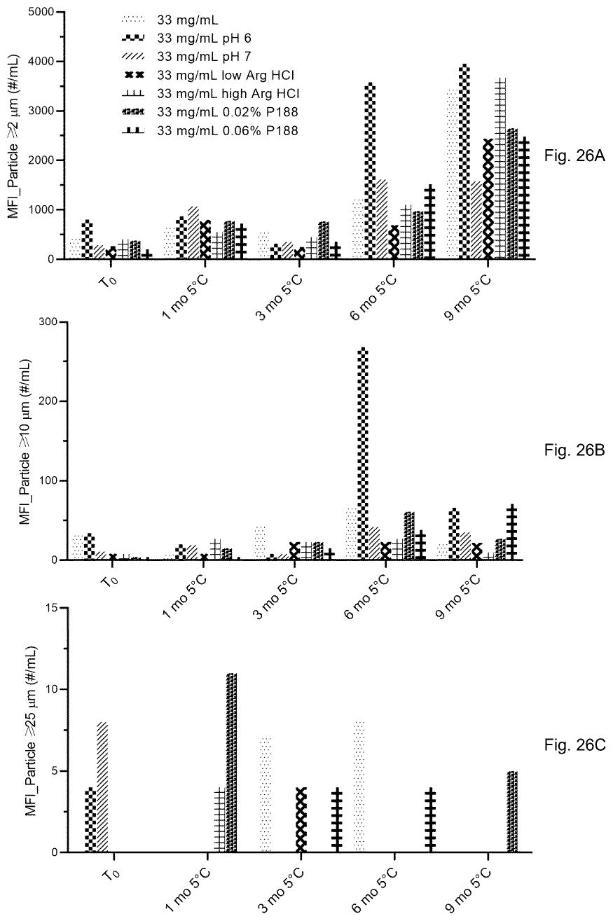

Thus, in some embodiments, the format of the heterodimeric fusion, e.g. HFUS1, is selected from:
(i) FcX-con-A/FcY-con-B (e.g. see FIG. 25);
(ii) FcX-con-B/FcY-con-A (e.g. see FIG. 25);
(iii) A-con-FcX/B-con-FcY (e.g. see FIG. 25);
(iv) B-con-FcX/A-con-FcY (e.g. see FIG. 25);
(v) Fab-FcX-con-A/Fab-FcY-con-B (e.g. see FIG. 25);
(vi) Fab-FcX-con-B/Fab-FcY-con-A;
(vii) A-con-FcX-con-A/B-con-FcY-con-B (e.g. see FIG. 25);
(viii) B-con-FcX-con-B/A-con-FcY-con-A;
(ix) FcX-con-B-L-A, and FcY, optionally FcY-con-B-L-A (e.g. see FIG. 25);
(x) FcY-con-B-L-A, and FcX, optionally FcX-con-B-L-A;
(xi) FcX-con-A-L-B, and FcY, optionally FcY-con-A-L-B; and
(xii) FcY-con-A-L-B, and FcX, optionally FcX-con-A-L-B,
wherein:
FcY is an immunoglobulin Fc region with “Fc Hole” amino acid mutations and/or modifications, optionally comprising a CH3 domain having the amino acid mutations Y349C:T366S:L368A:Y407V, or conservative substitutions thereof;
FcX is an Fc region with “Fc Knob” amino acid mutations and/or modifications, optionally comprising a CH3 domain having the amino acid mutations S354C:T366W, or conservative substitutions thereof; “con” is a connector polypeptide;
B is a Relaxin B chain or a variant thereof;
A is a Relaxin A chain or a variant thereof; and
L is a linker polypeptide, optionally with the amino acid sequence GGGSGGGSGG (SEQ ID NO: 60).

In another aspect, the heterodimeric fusion used in the formulation of the disclosure comprises
(i) X-B-L-A and Y, optionally Y-B-L-A; or
(ii) Y-B-L-A and X, optionally X-B-L-A,
wherein:
X and Y are heterodimerisation domains as described herein;
B is a Relaxin B chain or a variant thereof, e.g. a Relaxin-2 B chain or variant thereof;
A is a Relaxin A chain or a variant thereof, e.g. a Relaxin-2 A chain or variant thereof; and
L is a linker polypeptide, optionally with the amino acid sequence GGGSGGGSGG (SEQ ID NO: 60),
wherein X heterodimerises with Y, and wherein the heterodimeric fusion has Relaxin activity.

In yet another aspect, the heterodimeric fusion used in the formulation of the disclosure comprises
(i) X-A-L-B and Y, optionally Y-A-L-B or
(ii) Y-A-L-B and X, optionally X-A-L-B,
wherein:
   X and Y are heterodimerisation domains as described herein;
   A is a Relaxin A chain or a variant thereof, e.g. a Relaxin-2 A chain or variant thereof;
   B is a Relaxin B chain or a variant thereof, e.g. a Relaxin-2 B chain or variant thereof; and
   L is a linker polypeptide, optionally with the amino acid sequence GGGSGGGSGG (SEQ ID NO: 60),
wherein X heterodimerises with Y, and wherein the heterodimeric fusion has Relaxin activity.

In particular embodiments, the heterodimeric fusion comprises the fusion polypeptides Rlx011DD as set forth in SEQ ID NO: 11 and Rlx014DD as set forth in SEQ ID NO: 20. In particular embodiments, the heterodimeric fusion consists of the fusion polypeptides Rlx011DD as set forth in SEQ ID NO: 11 and Rix014DD as set forth in SEQ ID NO: 20 (termed “HFUS1” or “RELAX0023”). In alternative embodiments, the heterodimeric fusion comprises the fusion polypeptides Rlx013DD as set forth in SEQ ID NO: 17 and Rlx012DD as set forth in SEQ ID NO: 14.

In an aspect of the disclosure, the heterodimeric fusion used in the formulation of the disclosure, e.g. HFUS1, comprises a fusion polypeptide combination selected from the FcX and FcY combinations set forth in Table 2.

TABLE 2
Fusion polypeptide combinations in heterodimeric
fusions of the disclosure

| Heterodimeric | FcX (knob) fusion | FcY (hole) fusion |
|---|---|---|
| Fusion | polypeptide* | polypeptide* |
| RELAX0019 | Rlx011 | Rlx014 |
| RELAX0020 | Rlx013 | Rlx012 |
| RELAX0021 | Rlx011b | Rlx014b |
| RELAX0022 | Rlx012b | Rlx013b |
| HFUS1 (also termed | Rlx011DD | Rlx014DD |
| RELAX0023) | | |
| RELAX0024 | Rlx013DD | Rlx012DD |
| RELAX0034 | Rlx041H | Rlx014d |
| RELAX0039 | Rlx041M | Rlx014DD |
| RELAX0040 | Rlx041L | Rlx014DD |
| RELAX0041 | Rlx041H | Rlx014DD |
| RELAX0043 | Rlx041E | Rlx014DD |
| RELAX0046 | Rlx042R | Rlx014DD |
| RELAX0052 | Rlx044E | Rlx014DD |
| RELAX0053 | Rlx044H | Rlx014DD |
| RELAX0054 | Rlx028 | Rlx029 |
| RELAX0055 | Rlx030 | Rlx031 |
| RELAX0056 | Rlx026 | Rlx027 |
| RELAX0063 | Rlx052A | Rlx014DD |
| RELAX0069 | Rlx051M | Rlx014DD |
| RELAX0070 | Rlx051I | Rlx014DD |
| RELAX0071 | Rlx051Q | Rlx014DD |
| RELAX0072 | Rlx051A | Rlx014DD |
| RELAX0073 | Rlx051Y | Rlx014DD |
| RELAX0074 | Rlx051S | Rlx014DD |
| RELAX0075 | Rlx052I | Rlx014DD |
| RELAX0076 | Rlx052E | Rlx014DD |
| RELAX0081 | Rlx020 | Rlx021 |
| RELAX0082 | Rlx022 | Rlx023 |
| RELAX0083 | Rlx024 | Rlx025 |
| RELAX0084 | Rlx026 | Rlx014DD |
| RELAX0085 | Rlx011DD | Rlx027 |
| RELAX0086 | Rlx020 | Rlx014DD |
| RELAX0087 | Rlx011DD | Rlx021 |
| RELAX0088 | Rlx055 | FcH01 |
| RELAX0091 | Rlx062K | Rlx061H |
| RELAX0105 | Rlx020 | Rlx027 |
| RELAX0106 | Rlx022 | Rlx027 |
| RELAX0107 | Rlx024 | Rlx027 |
| RELAX0109 | Rlx020 | Rlx029 |
| RELAX0110 | Rlx022 | Rlx029 |
| RELAX0111 | Rlx024 | Rlx029 |
| RELAX0117 | Rlx076 | Rlx077 |
| RELAX0122 | Rlx055 | Rlx056 |
| RELAX0123 | Rlx011DD | Rlx014DDdel2aa |
| RELAX0124 | Rlx011DD | Rlx014DDdel3aa |
| RELAX0130** | R347Rlx011DD | R347Rlx014DD |

*The sequences of the fusion polypeptides listed are set forth in Table 3.
**In this particular embodiment the heterodimeric fusion is an IgG and comprises an additional polypeptide corresponding to the Light Chain set forth in SEQ ID NO: 54

In an aspect, there is provided a heterodimeric fusion comprising the fusion polypeptides set forth in SEQ ID NO: 11 and SEQ ID NO: 20.

In an alternative aspect, there is provided a heterodimeric fusion comprising the fusion polypeptides set forth in SEQ ID NO: 17 and SEQ ID NO: 14.

The fusion polypeptides used in the formulation of the disclosure may be produced by any method known in the art, and as described in WO2021/255127. In some embodiments, the fusion polypeptides used in the formulation of the disclosure are produced by recombinant expression of a nucleic acid molecule encoding a fusion polypeptide in a host cell.

Methods that are known to those skilled in the art can be used to construct expression vectors containing the nucleic acid molecules. Suitable vectors include, for example, plasmids, phagemids, phages or viral vectors.

Vectors containing the nucleic acid molecules may be transferred to a host cell by conventional techniques. Suitable host cells are known in the art. In some embodiments, the host cells are mammalian cells such as HEK293 cells or CHO cells.

The transfected cells may be cultured by conventional techniques to produce the fusion polypeptides used in the formulation of the disclosure.

Once a fusion polypeptide of the disclosure has been produced, for example by recombinant expression, it may be purified by any method known in the art. Exemplary protein purification techniques include chromatography (e.g. ion exchange, affinity and/or sizing column chromatography), centrifugation and differential solubility. WO2021/255127 provides isolated fusion polypeptides that have been separated from the cell culture, optionally by at least one purification step.

### Stable Pharmaceutical Formulation

The present disclosure provides a pharmaceutical formulation comprising a heterodimeric fusion, e.g. HFUS1, and a lipase-resistant surfactant, wherein the heterodimeric fusion comprises:
(i) a first heterodimerisation domain connected to at least one Relaxin A chain polypeptide or a variant thereof; and
(ii) a second heterodimerisation domain connected to at least one Relaxin B chain polypeptide or a variant thereof,
wherein the first heterodimerisation domain heterodimerises with the second heterodimerisation domain, and wherein the heterodimeric fusion has Relaxin activity.

In some embodiments, the pharmaceutical formulation further comprises a buffer. In some embodiments, the pharmaceutical formulation further comprises an excipient.

### Lipase-Resistant Surfactant

During the development of the pharmaceutical formulation of the disclosure, the present inventors identified the formation of visible particles in the HFUS1 formulations over time (see Example 3). Surprisingly, the inventors established that the presence of the surfactant PS80 was the cause of the particle formation. The inventors established that enzymatic hydrolysis of the ester bond of Polysorbate 80 (PS80) by a lipase (a host cell protein) present in the formulation (due to co-purification with HFUS1 during recombinant manufacture) was likely the cause of the degradation of PS80, which in turn resulted in the formation of impurities such as free fatty acids (FFA) which can serve as nucleus to trigger HFUS1 protein aggregation leading to particles. Additionally, degradation of PS80 over time can result in an effective reduction in the amount of PS80 in the formulation, adversely impacting the protective effect of the surfactant. The use of lipase-resistant surfactant, such as poloxamer 188 (P188) and D-α-Tocopherol polyethylene glycol succinate (TPGS), was able to mitigate the particle formation in the HFUS1 formulations.

“Surfactant” refers to a surface-active agent that lowers the surface tension of a liquid in which it is dissolved. Surfactants can be included in a pharmaceutical formulation for a variety of reasons including, for example, to prevent or control aggregation, particle formation or surface adsorption in liquid formulations or to prevent or control these phenomena during lyophilization or reconstitution of lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and to form micelles. Surfactants can be anionic, non-ionic, cationic, amphoteric, zwitterionic, and combinations thereof.

Surfactants are typically amphiphilic molecules that contain both hydrophilic and lipophilic groups. The hydrophile-lipophile balance (HLB) number can be used as a measure of the ratio between these groups and can have a value between 0-60, which defines the affinity of a surfactant for water or oil. Molecules with a HLB number greater than 10 have an affinity for water (hydrophilic) and molecules with a HLB number less than 10 have an affinity for oil (lipophilic). Non-ionic surfactants have HLB numbers ranging from 0-20.

The critical micelle concentration (CMC) is the concentration at and above which the surfactant forms micelles. Below the CMC, the surface tension decreases with increasing surfactant concentration. Above the CMC, additional surfactant added to the system forms micelles.

Lipase are a group of enzymes which can hydrolyse triglycerides into their component fatty acid and glycerol. Lipases include: lipoprotein lipase, Lipase 9, Phospholipase 2, Phospholipase 2A, pharyngeal lipase, hepatic lipase, pancreatic lipase, endothelial lipase, bile salt-dependent lipase, lysosomal lipase, hormone-sensitive lipase, gastric lipase, and lingual lipase.

In some embodiments, the lipase-resistant surfactant cannot be enzymatically hydrolysed by lipoprotein lipase, Lipase 9, Phospholipase 2, Phospholipase 2A, pharyngeal lipase, hepatic lipase, pancreatic lipase, endothelial lipase, bile salt-dependent lipase, lysosomal lipase, hormone-sensitive lipase, gastric lipase, or lingual lipase. In some embodiments, the lipase-resistant surfactant cannot be enzymatically hydrolysed by lipoprotein lipase.

In some embodiments, the lipase-resistant surfactant does not comprise an ester bond capable of being enzymatically hydrolysed by lipoprotein lipase, Lipase 9, Phospholipase 2, Phospholipase 2A, pharyngeal lipase, hepatic lipase, pancreatic lipase, endothelial lipase, bile salt-dependent lipase, lysosomal lipase, hormone-sensitive lipase, gastric lipase, or lingual lipase. In some embodiments, the lipase-resistant surfactant does not comprise an ester bond capable of being enzymatically hydrolysed by lipoprotein lipase.

In some embodiments, the lipase-resistant surfactant is a water-soluble non-ionic triblock copolymer formed by polyethylene oxide (PEO) and polypropylene oxide (PPO) blocks. In particular embodiments, the water-soluble nonionic triblock copolymer is poloxamer 188 (P188).

Poloxamer 188 (P188; CAS no. 9003-11-6) or Pluronic F68 is a nonionic triblock copolymer with a defined number of repeats of PEO and PPO with a molecular weight of approximately 7680-9510 Da. P188 has amphiphilic properties due to the presence of two hydrophilic side-chains (PPO) attached to a hydrophobic center core (PEO). Poloxamer has a HLB number of 29. See, Chen et al. (2022). Poloxamer 188 (P188), A Potential Polymeric Protective Agent for Central Nervous System Disorders: A Systematic Review. Curr Neuropharmacol. 20 (4): 799-808. P188 has a melting point from about 51° C. to about 53° C. P188 has a CMC of about 24 mg/ml to about 32 mg/ml at 37° C. See, Moghimi et al. (2004). Causative factors behind poloxamer 188 (Pluronic F68, Flocor™)-induced complement activation in human sera. A protective role against poloxamer-mediated complement activation by elevated serum lipoprotein levels. Biochimica et Biophysica Acta 1689; 103-113.

Alternatively, the lipase-resistant surfactant is D-α-Tocopherol polyethylene glycol succinate (TPGS). Formulations comprising TPGS have been described in WO 2022/101826.

As described in WO 2022/101826, TPGS (also referred to as tocophersolan) is a water-soluble synthetic derivative of natural a-tocopherol (vitamin E), formed by covalently joining tocopherol succinate, an ester formed through esterification of tocopherol and succinic acid, to a polyethylene glycol (PEG) moiety via an esterification reaction. The general structure of TPGS is:

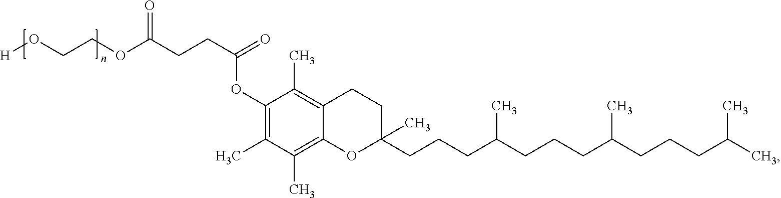

TPGS has amphiphilic properties due to the presence of a polar hydrophilic head (polyethylene glycol) and a lipophilic tail (phytyl chain of d-α-tocopherol). The TPGS surfactant can include PEG moieties with a variety of molecular weights. In one aspect, the PEG moiety of TPGS has a molecular weight of about 1000 Da and the TPGS molecule is referred to D-α-tocopheryl polyethylene glycol 1000 succinate (TPGS 1000). As used herein, the term TPGS includes TPGS 1000. TPGS 1000 has a HLB number of 13.2. See, Wu and Hopkins. (1999). Characteristics of D-alpha-tocopheryl PEG1000 succinate for applications as an absorption enhancer in drug delivery systems. Pharm Tech. 23:52-60. TPGS has a melting point from about 37° C. to about 41° C., or about 38° C., is stable at a pH from about 4.5 to about 7.5 and has a solubility in water of about 20% at 20° C. Vitamin E TPGS is a highly stable form of Vitamin E. See, PMC Isochem. (2015). Vitamin E TPGS:NF and Food Grade. Available at pmcisochem.fr/page/info-center. TPGS is generally regarded as safe (GRAS) and has been approved by the Federal Drug Administration (FDA) as an inactive ingredient for oral and topical formulations. TPGS 1000 has a CMC of 0.02% (w/w) at 37° C. See, Wu and Hopkins. (1999). Characteristics of D-alpha-tocopheryl PEG1000 succinate for applications as an absorption enhancer in drug delivery systems. Pharm Tech. 23:52-60.

TPGS formulations typically contain a mixture of monomers and dimers, where a monomer includes a single vitamin E molecule covalently joined to a water-soluble moiety, such as a polyethylene glycol (PEG), through a linker, in which the water-soluble moiety, e.g., PEG, has a free, unreacted, terminal reactive group, e.g., a free terminal hydroxyl group. A dimer includes two vitamin E molecules covalently joined to a water-soluble moiety, such as a polyethylene glycol (PEG), through one or more linkers, where both ends of the water-soluble moiety, e.g., both terminal hydroxyl groups of a PEG moiety, have reacted with a linker that is joined to a vitamin E molecule so that there are no free terminal reactive groups, e.g., hydroxyl groups. The monomers and dimers are formed during the esterification reaction. In some embodiments, the TPGS formulations includes at least about 70%, about 75%, about 80%, about 85%, about 86%, about 87%, about 88%, about 89% or about 90% TPGS monomer and less than about 30%, about 25%, about 20%, about 15%, about 14%, about 13%, about 12%, about 11% or about 10% TPGS dimer. In some embodiments, the TPGS formulation includes at least about 85% TPGS monomer and less than about 15% TPGS dimer.

In yet further embodiments, the lipase-resistant surfactant is selected from P188, TPGS, Kolliphor HS15, Kolliphor EL, Kolliphor RH40, PEG 300, PEG400, Brij 58 and Brij 35.

In some embodiments of any aspect of the disclosure, the concentration of the lipase-resistant surfactant is from 0.001% (w/v) to 1% (w/v), optionally 0.001% (w/v) to 0.5% (w/v). In some embodiments, the concentration of the lipase-resistant surfactant is from 0.005% (w/v) to 1% (w/v), optionally 0.005% (w/v) to 0.2% (w/v). In some embodiments, the concentration of the lipase-resistant surfactant is from 0.01% (w/v) to 0.15% (w/v). In some embodiments, the concentration of the lipase-resistant surfactant is from 0.01% (w/v) to 0.1% (w/v). In particular embodiments, the concentration of the lipase-resistant surfactant is from 0.02% (w/v) to 0.06% (w/v), optionally 0.02% (w/v), 0.03% (w/v), 0.04% (w/v), 0.05% (w/v) or 0.06% (w/v). In particular embodiments, the concentration of the lipase-resistant surfactant is 0.04% (w/v).

In some embodiments, the lipase-resistant surfactant is poloxamer 188 (P188). Thus, in some embodiments, the formulation of the disclosure comprises from about 0.01% (w/v) to about 0.1% (w/v) poloxamer 188 (P188). In some embodiments, the formulation of the disclosure comprises from about 0.02% (w/v) to about 0.06% (w/v) poloxamer 188 (P188). In some embodiments, the formulation of the disclosure comprises about 0.02% (w/v), about 0.03% (w/v), about 0.04% (w/v), about 0.05% (w/v), or about 0.06% (w/v) poloxamer 188 (P188). In particular embodiments, the formulation of the disclosure comprises from about 0.04% (w/v) poloxamer 188 (P188).

### Buffer and pH

During the development and optimization of the pharmaceutical formulation of the disclosure comprising a heterodimeric fusion as described herein, e.g. HFUS1, the present inventors have established that proteases are likely the cause of the fragmentation and clipping of HFUS1. This enzymatic activity is more likely to happen at lower pH where the proteases are most effective in cleaving the molecule. Therefore, the inventors established that the pH of the formulation can be important to minimise the chemical degradation impact such as fragmentation and amino acid (AA) clipping of the molecule, as demonstrated in the pH optimisation study (see Example 2).

In some embodiments of any aspects of the disclosure, the formulation comprises a buffer at a pH from about 3 to about 10, optionally about 5 to about 8. In some embodiments, the formulation comprises a buffer at a pH from about 5.5 to about 7.5. At the lower pH values, whilst protease activity is higher, the beneficial effects of the ionic excipient (e.g. arginine-HCl) and the lipase-resistant surfactant (e.g. P188) may still be obtained.

In some embodiments, the formulation has a pH in the range of about 6 to about 7. In some embodiments, the formulation has a pH of about 6.0, about 6.1, about 6.2, about 6.3, about 6.4, about 6.5, about 6.6, about 6.7, about 6.8, about 6.9 or about 7.0. In some embodiments, the formulation has a pH of about 6. In some embodiments, the formulation has a pH of about 7. In particular embodiments, the formulation has a pH of about 6.5.

As used herein, “buffer” refers to acid-base conjugate components that resist changes in pH, as known in the art.

In some embodiments of any aspects of the disclosure, the concentration of the buffer is from about 0.1 mM to about 100 mM, optionally about 5 mM, about 10 mM, about 15 nM, about 20 mM, about 25 mM, about 30 mM, about 35 mM, about 40 mM, about 45 mM, or about 50 mM. In some embodiments, the concentration of the buffer is from about 1 mM to about 50 mM, from about 10 to about 50 mM, or from about 10 to about 30 mM. In particular embodiments, the concentration of the buffer is from about 10 mM to about 30 mM.

In some embodiments, the concentration of the buffer is about 0.1 mM, about 0.5 mM, about 1 mM, about 5 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 35 mM, about 40 mM, about 45 mM, about 50 mM, about 75 mM or about 100 mM. In some embodiments, the concentration of the buffer is about 10 mM. In some embodiments, the concentration of the buffer is about 15 mM. In some embodiments, the concentration of the buffer is about 25 mM. In some embodiments, the concentration of the buffer is about 30 mM. In particular embodiments, the concentration of the buffer is about 15 mM, about 16 mM, about 17 mM, about 18 mM, about 19 mM, about 20 mM, about 21 mM, about 22 mM, about 23 mM, about 24 mM or about 25 mM. In particular embodiments, the concentration of the buffer is about 20 mM.

In some embodiments, the buffer is selected from acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof. In particular embodiments, the buffer is selected from histidine, citrate, acetate, phosphate, tris, succinate and mixtures thereof. In particular embodiments, the buffer is selected from a citrate buffer and a histidine buffer. In some embodiments, the buffer is a citrate buffer. In some embodiments, the buffer is a histidine buffer.

In particular embodiments, the buffer is a histidine, histidine hydrochloride or histidine/histidine hydrochloride buffer. In particular embodiments, the buffer is a histidine/histidine hydrochloride buffer (i.e. a combination of histidine and histidine hydrochloride). In one embodiment, the buffer is L-histidine/L-histidine hydrochloride monohydrate.

In some embodiments, the formulation of the disclosure comprises from about 10 mM to about 50 mM histidine/histidine hydrochloride buffer. In some embodiments, the formulation of the disclosure comprises from about 10 mM to about 30 mM histidine/histidine hydrochloride buffer. In some embodiments, the formulation of the disclosure comprises from about 15 mM to about 25 mM histidine/histidine hydrochloride buffer. In some embodiments, the formulation of the disclosure comprises from about 17 mM to about 23 mM histidine/histidine hydrochloride buffer. In some embodiments, the formulation of the disclosure comprises about 10 mM histidine/histidine hydrochloride buffer. In some embodiments, the formulation of the disclosure comprises about 15 mM histidine/histidine hydrochloride buffer. In some embodiments, the formulation of the disclosure comprises about 25 mM histidine/histidine hydrochloride buffer. In some embodiments, the formulation of the disclosure comprises about 30 mM histidine/histidine hydrochloride buffer. In particular embodiments, the formulation of the disclosure comprises about 20 mM histidine/histidine hydrochloride buffer.

### Excipient

During the development and optimisation of the pharmaceutical formulation of the disclosure comprising a heterodimeric fusion as described herein, e.g. HFUS1, the present inventors have demonstrated that the heterodimeric fusion, e.g. HFUS1, tends to self-associate (see Example 1). High levels of self-association of molecules can lead to the formation of soluble aggregates which can become precursors of insoluble, large-size aggregates, and particles eventually, significantly impacting the stability profile of the molecule. The optimisation of the pH, buffer and excipient used in the pharmaceutical formulation of the disclosure helped to reduce aggregation. Overall, the histidine-arginine HCl system was identified to provide the highest colloidal and conformational stability to the heterodimeric fusion, e.g. HFUS1 (see Example 2).

Thus, in some embodiments of any aspects of the disclosure, the formulation additionally comprises an excipient to reduce protein aggregation.

The pharmaceutical formulations of the disclosure may comprise one or more excipient(s). Pharmaceutically acceptable excipients are known in the art, see for instance Remington's Pharmaceutical Sciences (by Joseph P. Remington, 18th ed., Mack Publishing Co., Easton, PA), which is incorporated herein in its entirety.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

In some embodiments, the concentration of the excipient is from about 10 mM to about 500 mM, optionally 50 mM to 500 mM. In some embodiments, the concentration of the excipient is from 100 mM to 300 mM. In some embodiments, the concentration of the excipient is from 100 mM to 150 mM, from 150 mM to 200 mM, from 200 mM to 250 mM or from 250 mM to 300 mM. In particular embodiments, the concentration of the excipient is from 140 mM to 240 mM. In some embodiments, the concentration of the excipient is 140 mM. In some embodiments, the concentration of the excipient is 150 mM. In some embodiments, the concentration of the excipient is 160 mM. In some embodiments, the concentration of the excipient is 170 mM. In some embodiments, the concentration of the excipient is 180 mM. In some embodiments, the concentration of the excipient is 190 mM. In some embodiments, the concentration of the excipient is 200 mM. In some embodiments, the concentration of the excipient is 210 mM. In some embodiments, the concentration of the excipient is 220 mM. In some embodiments, the concentration of the excipient is 230 mM. In some embodiments, the concentration of the excipient is 240 mM. In particular embodiments, the concentration of the excipient is 190 mM.

In one aspect, the pharmaceutical formulation includes from about 1 mg/ml to about 50 mg/ml, about 5 mg/ml to about 25 mg/ml, about 10 mg/ml to about 20 mg/ml, or about 1 mg/ml to about 10 mg/ml of at least one amino acid as an excipient. In one aspect, the pharmaceutical formulation includes from about 1 mg/ml, about 5 mg/ml, about 10 mg/ml, about 15 mg/ml, about 20 mg/ml or about 25 mg/ml and up to about 30 mg/ml, about 35 mg/ml, about 40 mg/ml, about 45 mg/ml or about 50 mg/ml of at least one amino acid as an excipient. In one aspect, the pharmaceutical formulation includes about 1 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml or about 50 mg/ml of at least one amino acid as an excipient.

In some embodiments, the excipient is an ionic excipient. In some embodiments, the ionic excipient is an amino acid salt. “Amino acid salt” refers to a cationic or anionic form of an amino acid in combination with a counter ion with an opposite charge. In some embodiments, the amino acid salt is a pharmacologically acceptable salt. In some embodiments, the amino acid salt is an inorganic salt. In some embodiments, the amino acid salt is an organic salt. In some embodiments, the amino acid salt includes a sodium salt, potassium salt, calcium salt, magnesium salt, ammonium salt, hydrochloride salt, sulfate salt, nitrate salt, or phosphate salt. In some embodiments, the amino acid salt includes an organic acid salt such as acetate, citrate, maleate, malate, or oxalate salt. In some embodiments, the formulation includes an amino acid salt selected from a salt form of arginine, cysteine, glycine, lysine, ornithine, proline, alanine, glutamine, glutamic acid, histidine, valine or a combination thereof. In some embodiments, the amino acid salt includes arginine, lysine or histidine. In some embodiments, the amino acid salt is selected from an arginine salt or a lysine salt. In some embodiments, the ionic excipient is selected from arginine HCl or lysine HCl. In particular embodiments, the ionic excipient is arginine HCl.

In some embodiments, the formulation of the disclosure comprises from about 100 mM to about 300 mM arginine HCl. In particular embodiments, the formulation of the disclosure comprises from about 140 mM to about 240 mM arginine HCl. In some embodiments, the formulation of the disclosure comprises about 140 mM, about 150 mM, about 160 mM, about 170 mM, about 180 mM, about 190 mM, about 200 mM, about 210 mM, about 220 mM, about 230 mM, or about 240 mM arginine HCl. In particular embodiments, the formulation of the disclosure comprises from about 180 mM to about 200 mM arginine HCl. In particular embodiments, the formulation of the disclosure comprises about 190 mM arginine HCl.

### Additional Component

In some embodiments of any aspects of the disclosure, the formulation further comprises a sugar. The presence of a sugar can improve tonicity of the formulation. In particular embodiments, the concentration of the sugar is sufficient such that the formulation is isotonic or near isotonic. In some embodiments, the sugar is selected from sucrose, trehalose, fructose, glucose, mannose, melibiose, melezitose, raffinose, mannotriose, stachyose, sorbose, xylose, lactose, maltose, maltulose, iso-maltulose, lactulose, pullulan, dextrin, cyclodextrins, soluble starch, hydroxyethyl starch, water-soluble glucans, polyols such as trihydric or higher molecular weight sugar alcohols (e.g. glycerin, dextran, erythritol, glycerol, arabitol, xylitol, sorbitol, and mannitol) and mixtures thereof. In particular embodiments, the sugar is sucrose.

In some embodiments, the concentration of the sugar is from 1 mg/ml to about 300 mg/ml, about 10 mg/ml to about 200 mg/ml, about 50 mg/ml to about 100 mg/ml, or about 80 mg/ml to about 90 mg/ml. In some embodiments, the concentration of the sugar is about 30 mg/ml to about 90 mg/ml.

### Concentration of Heterodimeric Fusion

The present inventors evaluated the impact of heterodimeric fusion, e.g. HFUS1, concentration on stability of the pharmaceutical formulation. The heterodimeric fusion, e.g. HFUS1, showed good stability in formulations with varied heterodimeric fusion, e.g. HFUS1, concentrations (see Examples 4 to 6).

Thus, in some embodiments of any aspects of the disclosure, the concentration of heterodimeric fusion, e.g. HFUS1, in the formulation is from 0.1 to 100 mg/mL, optionally 0.2 to 50 mg/mL. In some embodiments, the concentration of heterodimeric fusion, e.g. HFUS1, in the formulation is from 0.25 mg/mL to 50 mg/mL. In some embodiments, the concentration of heterodimeric fusion, e.g. HFUS1, in the formulation is from 25 mg/ml to 35 mg/mL. In some embodiments, the concentration of heterodimeric fusion, e.g. HFUS1, in the formulation is 0.25 mg/mL, 0.5 mg/mL, 1 mg/mL, 1.1 mg/mL, 2 mg/ml, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL 19 mg/mL, 20 mg/mL, 21 mg/mL, 22 mg/mL, 23 mg/mL, 24 mg/mL, 25 mg/mL, 26 mg/mL, 27 mg/mL, 28 mg/mL 29, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42, mg/mL, 43 mg/mL, 44 mg/mL, 45 mg/mL, 46 mg/mL, 47 mg/mL, 48 mg/mL, 49 mg/mL or 50 mg/mL. In particular embodiments, the concentration of heterodimeric fusion, e.g. HFUS1, in the formulation is 0.25 mg/mL, 1 mg/mL, 5 mg/ml or 50 mg/mL. In some embodiments, the concentration of heterodimeric fusion, e.g. HFUS1, in the formulation is 0.25 mg/mL. In some embodiments, the concentration of heterodimeric fusion, e.g. HFUS1, in the formulation is 1 mg/mL. In some embodiments, the concentration of heterodimeric fusion, e.g. HFUS1, in the formulation is 1.1 mg/mL. In some embodiments, the concentration of heterodimeric fusion, e.g. HFUS1, in the formulation is 5 mg/mL. In some embodiments, the concentration of heterodimeric fusion, e.g. HFUS1, in the formulation is 30 mg/mL. In some embodiments, the concentration of heterodimeric fusion, e.g. HFUS1, in the formulation is 33 mg/mL. In some embodiments, the concentration of heterodimeric fusion, e.g. HFUS1, in the formulation is 50 mg/mL.

In some exemplary embodiments, the formulation comprises 0.2 mg/ml to 50 mg/mL of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 50 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 50 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.0.

In some embodiments, the formulation comprises 50 mg/mL of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 7.0.

In some embodiments, the formulation comprises 50 mg/ml of heterodimeric fusion (e.g. HFUS1), 20 mM histidine/histidine hydrochloride buffer, 140 mM arginine HCl, 0.04% (w/v) poloxamer 188 (P188), and wherein the formulation has a pH of 6.5.

In some embodiments, the formulation comprises 50 mg/ml of heterodimeric fusion (e.g. HFUS1), 20 mM histidine/histidine hydrochloride buffer, 240 mM arginine HCl, 0.04% (w/v) poloxamer 188 (P188), and wherein the formulation has a pH of 6.5.

In some embodiments, the formulation comprises 50 mg/ml of heterodimeric fusion (e.g. HFUS1), 20 mM histidine/histidine hydrochloride buffer, 190 mM arginine HCl, 0.02% (w/v) poloxamer 188 (P188), and wherein the formulation has a pH of 6.5.

In some embodiments, the formulation comprises 50 mg/ml of heterodimeric fusion (e.g. HFUS1), 20 mM histidine/histidine hydrochloride buffer, 190 mM arginine HCl, 0.06% (w/v) poloxamer 188 (P188), and wherein the formulation has a pH of 6.5.

In some embodiments, the formulation comprises 5 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 1.1 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 1 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 1 mg/ml of heterodimeric fusion (e.g. HFUS1), 20 mM histidine/histidine hydrochloride buffer, 140 mM arginine HCl, 0.04% (w/v) poloxamer 188 (P188), and wherein the formulation has a pH of 7.0.

In some embodiments, the formulation comprises 0.25 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 0.25 mg/ml of heterodimeric fusion (e.g. HFUS1), 20 mM histidine/histidine hydrochloride buffer, 140 mM arginine HCl, 0.04% (w/v) poloxamer 188 (P188), and wherein the formulation has a pH of 7.0.

In some embodiments, the formulation comprises 33 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.0.

In some embodiments, the formulation comprises 33 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 33 mg/mL of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 7.0.

In some embodiments, the formulation comprises 33 mg/ml of heterodimeric fusion (e.g. HFUS1), 20 mM histidine/histidine hydrochloride buffer, 150 mM arginine HCl, 0.04% (w/v) poloxamer 188 (P188), and wherein the formulation has a pH of 6.5.

In some embodiments, the formulation comprises 33 mg/ml of heterodimeric fusion (e.g. HFUS1), 20 mM histidine/histidine hydrochloride buffer, 230 mM arginine HCl, 0.04% (w/v) poloxamer 188 (P188), and wherein the formulation has a pH of 6.5.

In some embodiments, the formulation comprises 33 mg/ml of heterodimeric fusion (e.g. HFUS1), 20 mM histidine/histidine hydrochloride buffer, 190 mM arginine HCl, 0.02% (w/v) poloxamer 188 (P188), and wherein the formulation has a pH of 6.5.

In some embodiments, the formulation comprises 33 mg/ml of heterodimeric fusion (e.g. HFUS1), 20 mM histidine/histidine hydrochloride buffer, 190 mM arginine HCl, 0.06% (w/v) poloxamer 188 (P188), and wherein the formulation has a pH of 6.5.

In some embodiments, the formulation comprises 30 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.0.

In some embodiments, the formulation comprises 30 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 6.5.

In some embodiments, the formulation comprises 30 mg/ml of heterodimeric fusion (e.g. HFUS1), 20 mM histidine/histidine hydrochloride buffer, 190 mM arginine HCl, 0.04% (w/v) poloxamer 188 (P188), and wherein the formulation has a pH of 7.0.

In some embodiments, the formulation comprises 30 mg/ml of heterodimeric fusion (e.g. HFUS1), 20 mM histidine/histidine hydrochloride buffer, 150 mM arginine HCl, 0.04% (w/v) poloxamer 188 (P188), and wherein the formulation has a pH of 6.5.

In some embodiments, the formulation comprises 30 mg/ml of heterodimeric fusion (e.g. HFUS1), 20 mM histidine/histidine hydrochloride buffer, 230 mM arginine HCl, 0.04% (w/v) poloxamer 188 (P188), and wherein the formulation has a pH of 6.5.

In some embodiments, the formulation comprises 30 mg/ml of heterodimeric fusion (e.g. HFUS1), 20 mM histidine/histidine hydrochloride buffer, 190 mM arginine HCl, 0.02% (w/v) poloxamer 188 (P188), and wherein the formulation has a pH of 6.5.

In some embodiments, the formulation comprises 30 mg/ml of heterodimeric fusion (e.g. HFUS1), 20 mM histidine/histidine hydrochloride buffer, 190 mM arginine HCl, 0.06% (w/v) poloxamer 188 (P188), and wherein the formulation has a pH of 6.5.

### Stability

The present inventors have described some purposely designed studies to identify the root cause of instabilities with the heterodimeric fusion, e.g. HFUS1, used in the formulation of the disclosure, (see Example 1) and formulation development and optimization work to identify a stable liquid formulation for the molecule to meet its drug product shelf-life requirement (see Examples 2 to 6).

A “stable” formulation refers to a formulation in which the heterodimeric fusion, e.g. HFUS1, retains its physical stability, chemical stability or biological activity during storage. “Chemical stability” can be assessed by detecting and quantifying chemically altered forms of the heterodimeric fusion, e.g. HFUS1, including, for example, deamidation, including, for example, asparagine (Asn) deamidation; isomerization, including, for example, aspartate (Asp) isomerization; oxidation, including for example methionine (Met) oxidation; clipping/hydrolysis/fragmentation, including, for example, antibody hinge region fragmentation; succinimide formation; racemization; beta-elimination; glycation; adduct formation; disulfide scrambling; N-terminal extension; C-terminal processing; and glycosylation differences. “Physical stability” can be assessed by detecting and quantifying physically altered forms of the heterodimeric fusion, e.g. HFUS1, including, but not limited to, physically altered forms due to denaturation, aggregation, precipitation or particle formation, and surface adsorption.

Stability can be evaluated qualitatively or quantitatively in a variety of different ways, including, but not limited to, evaluation of aggregate formation (for example using size exclusion chromatography, by measuring turbidity, or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE; peptide map analysis; or by evaluating biological activity, for example, via an in vitro, in vivo or in situ assay indicative of heterodimeric fusion, e.g. HFUS1, activity.

A heterodimeric fusion, e.g. HFUS1, is “stable” in a pharmaceutical formulation, if the physical stability, chemical stability or biological activity of the heterodimeric fusion, e.g. HFUS1, at a given time is within about 0.1%, about 0.5%, about 1%, about 5%, about 10%, about 15%, about 20% or about 25% (within a standard of error) of the physical stability, chemical stability or biological activity of the heterodimeric fusion, e.g. HFUS1, exhibited at an initial time point, for example, at the time the pharmaceutical formulation was prepared.

Generally, stability is determined with respect to a selected temperature and a selected time-period. In some embodiments, the formulation is stable at a temperature of about 5° C.±10° C., about 5° C.±5° C. for at least about 2 weeks, about 1 month, about 2 months or about 3 months, about 6 months, about 9 months, about 12 months, about 24 months or about 36 months. In some embodiments, the formulation is stable at a temperature of about 25° C.±10° C., about 25° C.±5° C. for at least about 2 weeks, about 1 month, about 2 months or about 3 months, about 6 months, about 9 months, about 12 months, about 24 months or about 36 months. In some embodiments, the formulation is stable at a temperature of about 40° C.±10° C., about 40° C.±5° C. for at least about 2 weeks, about 1 month, about 2 months or about 3 months, about 6 months, about 9 months, about 12 months, about 24 months or about 36 months.

In some embodiments, the formulation is stable at a temperature from between about 2° C. to about 8° C., or at about 2° C., about 4° C., about 5° C., about 6° C. or about 8° C. for at least about 2 weeks, about 1 month, about 3 months, or about 6 months and up to about 9 months, about 12 months, about 24 months or about 36 months. In some embodiments, the formulation is stable at a temperature from between about 22° C. to about 28° C., or at about 22° C., about 24° C., about 25° C., about 26° C. or about 28° C. for at least about 2 weeks, about 1 month, about 3 months, or about 6 months and up to about 9 months, about 12 months, about 24 months or about 36 months. In some embodiments, the formulation is stable at a temperature from between about 37° C. to about 43° C., or at about 37° C., about 39° C., about 40° C., about 41° C. or about 43° C. for at least about 2 weeks, about 1 month, about 3 months, or about 6 months and up to about 9 months, about 12 months, about 24 months or about 36 months.

In some embodiments, the formulation is stable at 40° C. for up to about 3 months. In some embodiments, the formulation is stable at 40° C. for up to about 6 months. In some embodiments, the formulation is stable at 25° C. for up to about 6 months. In some embodiments, the formulation is stable at 25° C. for up to about 12 months. In some embodiments, the formulation is stable at 25° C. for up to about 24 months. In some embodiments, the formulation is stable at 5° C. for up to about 6 months. In some embodiments, the formulation is stable at 5° C. for up to about 12 months. In some embodiments, the formulation is stable at 5° C. for up to about 24 months. In some embodiments, the formulation is stable at 5° C. for up to about 36 months.

In some embodiments, the heterodimeric fusion, e.g. HFUS1, in the pharmaceutical formulation is chemically stable. In some embodiments, there is an increase of less than about 25%, about 20%, about 15%, about 10% or about 5% in chemically altered forms of the heterodimeric fusion, e.g. HFUS1, in the pharmaceutical formulation when stored for at least about 2 weeks, about 1 month, about 2 months, about 3 months, about 6 months, about 9 months, about 12 months, about 24 months or about 36 months at a temperature of about 40° C., about 25° C., about 2° C. to about 8° C. or about 5° C. In some embodiments, there is less than about 25%, about 20%, about 15%, about 10% or about 5% increase in one or more chemically altered forms of the heterodimeric fusion, e.g. HFUS1, including, for example, chemically altered forms due to deamidation, including, for example, asparagine (Asn) deamidation; oxidation, including for example methionine, cysteine, histidine, tyrosine, tryptophan or phenylalanine oxidation; intra- and inter-residue cyclization (aspartic and glutamic acid, asparagine, glutamine, N-terminal dipeptidyl motifs); clipping/hydrolysis/fragmentation; β-elimination; glycation; and disulfide scrambling.

In some embodiments, the heterodimeric fusion, e.g. HFUS1, in the pharmaceutical formulation is physically stable. In some embodiments, there is an increase of less than about 25%, about 20%, about 15%, about 10% or about 5% in physically altered forms of the heterodimeric fusion, e.g. HFUS1, in the pharmaceutical formulation when stored for at least about 2 weeks, about 1 month, about 2 months, about 3 months or about 6 months and up to about 9 months, about 12 months, about 24 months or about 36 months at a temperature of about 40° C., about 25° C., about 2° C. to about 8° C. or about 5° C. In one aspect, there is less than about 25%, about 20%, about 15%, about 10% or about 5% increase in one or more physically altered forms of the heterodimeric fusion, e.g. HFUS1, including, for example, physically altered forms due to denaturation, aggregation, precipitation, or particulate formation.

In some embodiments, the heterodimeric fusion, e.g. HFUS1, in the pharmaceutical formulation is biologically stable. In some embodiment, there is a decrease of less than about 25%, about 20%, about 15%, about 10% or about 5% in a biological activity of the heterodimeric fusion, e.g. HFUS1, in the pharmaceutical formulation when stored for at least about 2 weeks, about 1 month, about 2 months, about 3 months or about 6 months and up to about 9 months, about 12 months, about 24 months or about 36 months at a temperature of about 40° C., about 25° C., about 2° C. to about 8° C. or about 5° C.

In some embodiments, the concentration of lipase-resistant surfactant in the pharmaceutical formulation remains stable during the duration of storage. In some embodiments, there is a decrease of less than about 25%, about 20%, about 15%, about 10% or about 5% of the concentration of lipase-resistant surfactant in the pharmaceutical formulation when stored for at least about 2 weeks, about 1 month, about 2 months, about 3 months, about 6 months, about 9 months, about 12 months, about 24 months or about 36 months at a temperature of about 40° C., about 25° C., about 2° C. to about 8° C. or about 5° C.

In some embodiments, the stability of a pharmaceutical formulation is evaluated by measuring the amount of particle impurities. Particle impurities can include visible, subvisible and submicron impurities. Visible impurities have a diameter of greater than about 100 μm or about 150 μm and can be detected by visual inspection. Sub-visible particles generally range in size from about 1 μm to about 100 μm or about 150 μm. Submicron particles have a diameter of less than about 1 μm. Sub-visible particles generally pose the greatest risk when present in pharmaceutical formulations, particularly when present in pharmaceutical formulations for parenteral administration, including subcutaneous, intravenous or intramuscular administration, due, in some cases, to the ability of sub-visible particles to elicit an adverse immunogenic response. Sub-visible particles with a diameter of greater than or equal to about 10 μm or greater than or equal to about 5 μm can block blood vessels in the lungs following vascular infusion. Methods for detecting and quantifying particulate impurities are known and include Dynamic Light Scattering (DLS) or Static Light Scattering (SLS), Nanoparticle Tracking Analysis (NTA), light microscopy, Electrical Sensing Zone (ESZ), Flow-Imaging Technology, Resonant Mass measurement, Electron Microscopy, Fourier Transform Infrared (FTIR) Microscopy, and Raman Microscopy. In some embodiments, sub-visible particles are detected using Micro Flow Imaging (MFI). In some embodiments, sub-visible particles are detected using a light obscuration method, for example a High Accuracy Products (HIAC) system, for example a HIAC system model 9703 equipped with a HRLD150 sensor.

In some embodiments of any aspect of the disclosure, the pharmaceutical formulation comprises less than about 10,000, about 6000, about 5,000, about 1,000, about 750, about 600, about 500, about 250, about 150, about 100, or about 50 particles/mL greater than about 2 μm, about 5 μm, about 10 μm, about 15 μm, about 20 μm or about 25 μm diameter. In some embodiments, the pharmaceutical formulation comprises less than about 10,000, about 6000, about 5,000, about 1,000, about 750, about 600, about 500, about 250, about 150, about 100, or about 50 particles/mL greater than 2 μm, 5 μm or 10 μm diameter. In some embodiments, the pharmaceutical formulation comprises less than about 10,000, about 6000, about 5,000, about 1,000, about 750, about 600, about 500, about 250, about 150, about 100, or about 50 particles/mL greater than 2 μm diameter. In some embodiments, the pharmaceutical formulation comprises less than about 10,000, about 6000, about 5,000, about 1,000, about 750, about 600, about 500, about 250, about 150, about 100, or about 50 particles/mL greater than 10 μm diameter.

The pharmaceutical formulation may comprise subvisible particles counts within the USP limits (particle size of ≥10 μm and ≥25 μm not exceeding 6000 and 600 respectively; Pharmacopeia US. 2014. USP 787 and 788). Thus, in particular embodiments, the pharmaceutical formulation comprises less than about 6000 particles/mL greater than about 10 μm diameter. In particular embodiments, the pharmaceutical formulation comprises less than about 600 particles/mL greater than about 25 μm diameter.

In some embodiments, the pharmaceutical formulation comprises less than about 6,000 or 5,000 particles/mL greater than about 2 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 6,000 or 5,000 particles/mL greater than about 2 μm in diameter when stored at a temperature of about 40° C.±10° C. for up to about 3 months. In some embodiments, the pharmaceutical formulation comprises less than about 6,000 or 5,000 particles/mL greater than about 2 μm in diameter when stored at a temperature of about 40° C.±10° C. for up to about 6 months. In some embodiments, the pharmaceutical formulation comprises less than about 6,000 or 5,000 particles/mL greater than about 2 μm in diameter when stored at a temperature of about 30° C.±10° C. for up to about 3 months. In some embodiments, the pharmaceutical formulation comprises less than about 6,000 or 5,000 particles/mL greater than about 2 μm in diameter when stored at a temperature of about 30° C.±10° C. for up to about 6 months. In some embodiments, the pharmaceutical formulation comprises less than about 6,000 or 5,000 particles/mL greater than about 2 μm in diameter when stored at a temperature of about 25° C.±10° C. for up to about 6 months. In some embodiments, the pharmaceutical formulation comprises less than about 6,000 or 5,000 particles/mL greater than about 2 μm in diameter when stored at a temperature of about 25° C.±5° C. for up to about 12 months. In some embodiments, the pharmaceutical formulation comprises less than about 6,000 or 5,000 particles/mL greater than about 2 μm in diameter when stored at a temperature of about 20° C.±10° C. for up to about 6 months. In some embodiments, the pharmaceutical formulation comprises less than about 6,000 or 5,000 particles/mL greater than about 2 μm in diameter when stored at a temperature of about 20° C.±5° C. for up to about 12 months. In some embodiments, the pharmaceutical formulation comprises less than about 6,000 or 5,000 particles/mL greater than about 2 μm in diameter when stored at a temperature of about 5° C.±3° C. for up to about 12 months. In some embodiments, the pharmaceutical formulation comprises less than about 6,000 or 5,000 particles/mL greater than about 2 μm in diameter when stored at a temperature of about 5° C.±3° C. for up to about 24 months. In some embodiments, the pharmaceutical formulation comprises less than about 6,000 or 5,000 particles/mL greater than about 2 μm in diameter when stored at a temperature of about 5° C.±3° C. for up to about 36 months.

In some embodiments, the pharmaceutical formulation comprises less than about 1,000 particles/mL greater than about 2 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 1,000 particles/mL greater than about 2 μm in diameter when stored at a temperature of about 40° C.±10° C. for up to about 3 months. In some embodiments, the pharmaceutical formulation comprises less than about 1,000 particles/mL greater than about 2 μm in diameter when stored at a temperature of about 40° C.±10° C. for up to about 6 months. In some embodiments, the pharmaceutical formulation comprises less than about 1,000 particles/mL greater than about 2 μm in diameter when stored at a temperature of about 30° C.±10° C. for up to about 3 months. In some embodiments, the pharmaceutical formulation comprises less than about 1,000 particles/mL greater than about 2 μm in diameter when stored at a temperature of about 30° C.±10° C. for up to about 6 months. In some embodiments, the pharmaceutical formulation comprises less than about 1,000 particles/mL greater than about 2 μm in diameter when stored at a temperature of about 25° C.±10° C. for up to about 6 months. In some embodiments, the pharmaceutical formulation comprises less than about 1,000 particles/mL greater than about 2 μm in diameter when stored at a temperature of about 25° C.±5° C. for up to about 12 months. In some embodiments, the pharmaceutical formulation comprises less than about 1,000 particles/mL greater than about 2 μm in diameter when stored at a temperature of about 20° C.±10° C. for up to about 6 months. In some embodiments, the pharmaceutical formulation comprises less than about 1,000 particles/mL greater than about 2 μm in diameter when stored at a temperature of about 20° C.±5° C. for up to about 12 months. In some embodiments, the pharmaceutical formulation comprises less than about 1,000 particles/mL greater than about 2 μm in diameter when stored at a temperature of about 5° C.±3° C. for up to about 12 months. In some embodiments, the pharmaceutical formulation comprises less than about 1,000 particles/mL greater than about 2 μm in diameter when stored at a temperature of about 5° C.±3° C. for up to about 24 months. In some embodiments, the pharmaceutical formulation comprises less than about 1,000 particles/mL greater than about 2 μm in diameter when stored at a temperature of about 5° C.±3° C. for up to about 36 months.

In some embodiments, the pharmaceutical formulation comprises less than about 6,000 or 5,000 particles/mL greater than about 5 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 6,000 or 5,000 particles/mL greater than about 5 μm in diameter when stored at a temperature of about 40° C.±10° C. for up to about 3 months. In some embodiments, the pharmaceutical formulation comprises less than about 6,000 or 5,000 particles/mL greater than about 5 μm in diameter when stored at a temperature of about 40° C.±10° C. for up to about 6 months. In some embodiments, the pharmaceutical formulation comprises less than about 6,000 or 5,000 particles/mL greater than about 5 μm in diameter when stored at a temperature of about 30° C.±10° C. for up to about 3 months. In some embodiments, the pharmaceutical formulation comprises less than about 6,000 or 5,000 particles/mL greater than about 5 μm in diameter when stored at a temperature of about 30° C.±10° C. for up to about 6 months. In some embodiments, the pharmaceutical formulation comprises less than about 6, 000 or 5,000 particles/mL greater than about 5 μm in diameter when stored at a temperature of about 25° C.±10° C. for up to about 6 months. In some embodiments, the pharmaceutical formulation comprises less than about 6,000 or 5,000 particles/mL greater than about 5 μm in diameter when stored at a temperature of about 25° C.±5° C. for up to about 12 months. In some embodiments, the pharmaceutical formulation comprises less than about 6,000 or 5,000 particles/mL greater than about 5 μm in diameter when stored at a temperature of about 20° C.±10° C. for up to about 6 months. In some embodiments, the pharmaceutical formulation comprises less than about 6,000 or 5,000 particles/mL greater than about 5 μm in diameter when stored at a temperature of about 20° C.±5° C. for up to about 12 months. In some embodiments, the pharmaceutical formulation comprises less than about 6,000 or 5,000 particles/mL greater than about 5 μm in diameter when stored at a temperature of about 5° C.±3° C. for up to about 12 months. In some embodiments, the pharmaceutical formulation comprises less than about 6,000 or 5,000 particles/mL greater than about 5 μm in diameter when stored at a temperature of about 5° C.±3° C. for up to about 24 months. In some embodiments, the pharmaceutical formulation comprises less than about 6,000 or 5,000 particles/mL greater than about 5 μm in diameter when stored at a temperature of about 5° C.±3° C. for up to about 36 months.

In some embodiments, the pharmaceutical formulation comprises less than about 1,000 particles/mL greater than about 5 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 1,000 particles/mL greater than about 5 μm in diameter when stored at a temperature of about 40° C.±10° C. for up to about 3 months. In some embodiments, the pharmaceutical formulation comprises less than about 1,000 particles/mL greater than about 5 μm in diameter when stored at a temperature of about 40° C.±10° C. for up to about 6 months. In some embodiments, the pharmaceutical formulation comprises less than about 1,000 particles/mL greater than about 5 μm in diameter when stored at a temperature of about 30° C.±10° C. for up to about 3 months. In some embodiments, the pharmaceutical formulation comprises less than about 1,000 particles/mL greater than about 5 μm in diameter when stored at a temperature of about 30° C.±10° C. for up to about 6 months. In some embodiments, the pharmaceutical formulation comprises less than about 1,000 particles/mL greater than about 5 μm in diameter when stored at a temperature of about 25° C.±10° C. for up to about 6 months. In some embodiments, the pharmaceutical formulation comprises less than about 1,000 particles/mL greater than about 5 μm in diameter when stored at a temperature of about 25° C.±5° C. for up to about 12 months. In some embodiments, the pharmaceutical formulation comprises less than about 1,000 particles/mL greater than about 5 μm in diameter when stored at a temperature of about 20° C.±10° C. for up to about 6 months. In some embodiments, the pharmaceutical formulation comprises less than about 1,000 particles/mL greater than about 5 μm in diameter when stored at a temperature of about 20° C.±5° C. for up to about 12 months. In some embodiments, the pharmaceutical formulation comprises less than about 1,000 particles/mL greater than about 5 μm in diameter when stored at a temperature of about 5° C.±3° C. for up to about 12 months. In some embodiments, the pharmaceutical formulation comprises less than about 1,000 particles/mL greater than about 5 μm in diameter when stored at a temperature of about 5° C.±3° C. for up to about 24 months. In some embodiments, the pharmaceutical formulation comprises less than about 1,000 particles/mL greater than about 5 μm in diameter when stored at a temperature of about 5° C.±3° C. for up to about 36 months.

In some embodiments, the pharmaceutical formulation comprises less than about 6,000 or 5,000 particles/mL greater than about 10 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 6,000 or 5,000 particles/mL greater than about 10 μm in diameter when stored at a temperature of about 40° C.±10° C. for up to about 3 months. In some embodiments, the pharmaceutical formulation comprises less than about 6,000 or 5,000 particles/mL greater than about 10 μm in diameter when stored at a temperature of about 40° C.±10° C. for up to about 6 months. In some embodiments, the pharmaceutical formulation comprises less than about 6,000 or 5,000 particles/mL greater than about 10 μm in diameter when stored at a temperature of about 30° C.±10° C. for up to about 3 months. In some embodiments, the pharmaceutical formulation comprises less than about 6,000 or 5,000 particles/mL greater than about 10 μm in diameter when stored at a temperature of about 30° C.±10° C. for up to about 6 months. In some embodiments, the pharmaceutical formulation comprises less than about 6,000 or 5,000 particles/mL greater than about 10 μm in diameter when stored at a temperature of about 25° C.±10° C. for up to about 6 months. In some embodiments, the pharmaceutical formulation comprises less than about 6,000 or 5,000 particles/mL greater than about 10 μm in diameter when stored at a temperature of about 25° C.±5° C. for up to about 12 months. In some embodiments, the pharmaceutical formulation comprises less than about 6,000 or 5,000 particles/mL greater than about 10 μm in diameter when stored at a temperature of about 20° C.±10° C. for up to about 6 months. In some embodiments, the pharmaceutical formulation comprises less than about 6,000 or 5,000 particles/mL greater than about 10 μm in diameter when stored at a temperature of about 20° C.±5° C. for up to about 12 months. In some embodiments, the pharmaceutical formulation comprises less than about 6,000 or 5,000 particles/mL greater than about 10 μm in diameter when stored at a temperature of about 5° C.±3° C. for up to about 12 months. In some embodiments, the pharmaceutical formulation comprises less than about 6,000 or 5,000 particles/mL greater than about 10 μm in diameter when stored at a temperature of about 5° C.±3° C. for up to about 24 months. In some embodiments, the pharmaceutical formulation comprises less than about 6,000 or 5,000 particles/mL greater than about 10 μm in diameter when stored at a temperature of about 5° C.±3° C. for up to about 36 months.

In some embodiments, the pharmaceutical formulation comprises less than about 1,000 particles/mL greater than about 10 μm diameter when stored for at least about 2 weeks, about 1 month, about 2 months, about 3 months, about 6 months, about 9 months, about 12 months, about 24 months or about 36 months at a temperature of about 40° C.±10° C., about 30° C.±10° C., about 25° C.±10° C., about 20° C.±10° C. or about 5° C.±3° C. In some embodiments, the pharmaceutical formulation comprises less than about 1,000 particles/mL greater than about 10 μm in diameter when stored at a temperature of about 40° C.±10° C. for up to about 3 months. In some embodiments, the pharmaceutical formulation comprises less than about 1,000 particles/mL greater than about 10 μm in diameter when stored at a temperature of about 40° C.±10° C. for up to about 6 months. In some embodiments, the pharmaceutical formulation comprises less than about 1,000 particles/mL greater than about 10 μm in diameter when stored at a temperature of about 30° C.±10° C. for up to about 3 months. In some embodiments, the pharmaceutical formulation comprises less than about 1,000 particles/mL greater than about 10 μm in diameter when stored at a temperature of about 30° C.±10° C. for up to about 6 months. In some embodiments, the pharmaceutical formulation comprises less than about 1,000 particles/mL greater than about 10 μm in diameter when stored at a temperature of about 25° C.±10° C. for up to about 6 months. In some embodiments, the pharmaceutical formulation comprises less than about 1,000 particles/mL greater than about 10 μm in diameter when stored at a temperature of about 25° C.±5° C. for up to about 12 months. In some embodiments, the pharmaceutical formulation comprises less than about 1,000 particles/mL greater than about 10 μm in diameter when stored at a temperature of about 20° C.±10° C. for up to about 6 months. In some embodiments, the pharmaceutical formulation comprises less than about 1,000 particles/mL greater than about 10 μm in diameter when stored at a temperature of about 20° C.±5° C. for up to about 12 months. In some embodiments, the pharmaceutical formulation comprises less than about 1,000 particles/mL greater than about 10 μm in diameter when stored at a temperature of about 5° C.±3° C. for up to about 12 months. In some embodiments, the pharmaceutical formulation comprises less than about 1,000 particles/mL greater than about 10 μm in diameter when stored at a temperature of about 5° C.±3° C. for up to about 24 months. In some embodiments, the pharmaceutical formulation comprises less than about 1,000 particles/mL greater than about 10 μm in diameter when stored at a temperature of about 5° C.±3° C. for up to about 36 months.

### Therapeutic Methods

The present disclosure encompasses therapies which involve administering the pharmaceutical formulation of the disclosure to an animal, in particular a mammal, for instance a human, for preventing, treating, or ameliorating symptoms associated with a disease, disorder, or infection.

Accordingly, the pharmaceutical formulation of the disclosure may be used in therapy, for example for treating a disease or disorder. Also provided is a method of treating a disease or disorder comprising administering to a subject or patient in need thereof a therapeutically effective amount of the fusion polypeptides as described herein, e.g. HFUS1. The use or method may comprise administering a therapeutically effective schedule that has less frequent doses of the fusion polypeptides as described herein, e.g. HFUS1, than the therapeutically effective dosing schedule of a wild-type Relaxin molecule.

The heterodimeric fusions described herein have been described as useful for treating various disorders, including heart failure and heart failure with pulmonary hypertension (see WO2023/111112, incorporated herein by reference). Thus, it will be understood that the pharmaceutical formulation of the disclosure may be used in the treatment of cardiovascular diseases, for example for the treatment of heart failure, and more specifically for the treatment of heart failure with pulmonary hypertension.

The pharmaceutical formulation of the disclosure may also be used in the treatment of kidney disease, lung disease and fibrotic disorders, for example fibrotic disorders of the kidney, heart, lung and liver, and in wound healing (Sherwood O D (2004) Endocrine Reviews 25 (2): 205-234). The fusion polypeptides as described herein, e.g. HFUS1, may also be used in the reversal of insulin resistance in diabetic patients (Bonner J S et al. (2013) Diabetes 62 (9): 3251-3260). The pharmaceutical formulation of the disclosure may also be used in various forms of pulmonary hypertension. The pharmaceutical formulation of the disclosure may also be used in disorders that are a result of or a cause of arterial stiffness, reduced arterial elasticity, reduced arterial compliance and distensibility including hypertension, kidney disease, peripheral arterial disease, carotid and cerebrovascular disease (i.e. stroke and dementia), diabetes, microvascular disease resulting in end organ damage, coronary artery disease, and heart failure.

The present disclosure encompasses methods of treating a subject with heart failure, particularly heart failure with pulmonary hypertension, by administering a pharmaceutical formulation as described herein, as well as uses of said pharmaceutical formulations for use in said methods. In particular, the subject may be an animal, particularly a mammal, more particularly a human.

The use or method may comprise administering a therapeutically effective schedule that has less frequent doses of the heterodimeric fusions/fusion polypeptides as described herein, e.g. HFUS1, than the therapeutically effective dosing schedule of a wild-type Relaxin molecule.

As used herein, the term “heart failure” includes acute heart failure, chronic heart failure (CHF) and acute decompensated heart failure (ADHF). The term “heart failure” may also include more specific diagnoses such as heart failure with preserved ejection fraction (HFpEF), heart failure with mid-range ejection fraction or heart failure with reduced ejection fraction (HFrEF). This may also include heart failure due to hypertrophic cardiomyopathy or dilated cardiomyopathy.

As used herein, the term “pulmonary hypertension” may be defined as a subject with a mean Pulmonary Arterial Pressure of about 20 mmHg or greater, optionally 25 mmHg or greater, typically when the subject is at rest. It may also be defined as a mean Pulmonary Arterial Pressure of about 30 mmHg or greater, typically when the subject is or has recently been exercising. Thus, the subject may have a mean Pulmonary Arterial Pressure in the range of about 20 mmHg to about 30 mmHg, optionally about 25 mmHg to about 30 mmHg, or greater. Alternatively or additionally, the subject may have:
a. a Right Ventricular Systolic Pressure of about 40 mmHg or greater;
b. a pulmonary artery wedge pressure (PAWP) greater than 15 mmHg; and/or
c. a Pulmonary Vascular Resistance of:
   i. less than 3.0 wood units; or
   ii. 3.0 or more wood units.

Thus, in some cases, the pulmonary hypertension may be classified as Group 2 pulmonary hypertension, as defined by the World Health Organisation. This may also be termed as “Heart Failure with Pulmonary Hypertension due to Left Heart Disease”. In other cases, the pulmonary hypertension may be classified as Group 1 pulmonary arterial hypertension, as defined by the World Health Organisation (see Ryan et al., 2012, Pulm. Circ. 2 (1): 107-121).

Parameters of pulmonary hypertension and heart failure may be measured or estimated using techniques known in the art. For instance, these include echocardiography, pulmonary artery catheter and implantable monitoring device. In certain embodiments, the subject may have been fitted with a blood pressure monitoring device, optionally a pulmonary artery pressure monitoring device, as are known in the art. In particular embodiments, the pulmonary artery pressure monitoring device is a CardioMEMS pressure monitoring device. Typically, the device is fitted prior to treatment with a heterodimeric fusion as described herein, e.g. HFUS1. Alternatively, the subject is fitted with the device during or after the period of treatment.

As used herein, the term “heart failure with pulmonary hypertension” refers to the subset of heart failure subjects who simultaneously suffer from pulmonary hypertension (HF+PH subjects).

“Treatment” refers to the amelioration and/or elimination of one or more symptoms or causes of the target disease. In some embodiments, this may involve modulating the levels of one or more biological markers or functions to within a non-diseased range (as compared against a healthy cohort). For instance, the pharmaceutical formulations of the disclosure may reduce Pulmonary Vascular Resistance (PVR) in a subject. For example, PVR may be reduced after treatment by at least 1% to 10%, 1% to 20%, 1% to 30%, 1% to 40% or 1% to 50% or greater as compared to baseline PVR (prior to administration to the subject of the heterodimeric fusion as described herein, e.g. HFUS1,). Thus, the pharmaceutical formulations of the disclosure may reduce PVR in a subject by at least 1%, 2%, 3%, 4%, 5%, 6%, 7%, 8%, 9%, 10%, 20%, 30%, 40%, 50% or greater, as compared to baseline PVR (prior to administration to the subject of the pharmaceutical formulation of the disclosure). In addition, or alternatively, the pharmaceutical formulations of the disclosure may reduce mean Pulmonary Artery Pressure (mPAP) in a subject. For example, mPAP may be reduced by at least 1 mmHg to 15 mmHg or greater. Thus, the pharmaceutical formulations of the disclosure may reduce mean Pulmonary Artery Pressure in a subject by at least 1 mmHg, at least 2 mmHg, at least 3 mmHg, at least 4 mmHg, at least 5 mmHg, at least 6 mmHg, at least 7 mmHg, at least 8 mmHg, at least 9 mmHg, at least 10 mmHg, at least 11 mmHg, at least 12 mmHg, at least 13 mmHg, at least 14 mmHg or at least 15 mmHg or greater. Equally, the pharmaceutical formulations of the disclosure may reduce estimated Pulmonary Artery Diastolic Pressure (ePAD) in a subject. For example, ePAD may be reduced by at least 1 mmHg to 15 mmHg or greater. Thus, the pharmaceutical formulations of the disclosure may reduce estimated Pulmonary Artery Diastolic Pressure in a subject by at least 1 mmHg, at least 2 mmHg, at least 3 mmHg, at least 4 mmHg, at least 5 mmHg, at least 6 mmHg, at least 7 mmHg, at least 8 mmHg, at least 9 mmHg, at least 10 mmHg, at least 11 mmHg, at least 12 mmHg, at least 13 mmHg, at least 14 mmHg or at least 15 mmHg or greater. In addition, or alternatively, the pharmaceutical formulations of the disclosure may increase percentage ejection fraction (EF %) in a subject, as a measure of cardiac output. For example, EF % may increase by at least 1% to 10%, 1% to 20%, 1% to 30%, 1% to 40% or 1% to 50% or greater. Thus, the pharmaceutical formulations of the disclosure may increase percentage ejection fraction (EF %) in a subject by at least 1%, 2%, 3%, 4%, 5%, 6%, 7%, 8%, 9%, 10%, 20%, 30%, 40%, 50% or greater. In addition, or alternatively, the pharmaceutical formulations of the disclosure may, in a subject:
(a) increase stroke volume (SV) of the heart;
(b) decrease systemic vascular resistance (SVR) and/or increase estimated glomerular filtration rate (eGFR);
(c) increase ejection fraction; and/or
(d) increase cardiac output;
as compared to baseline levels pre-administration. A combined decrease in SVR and increase in eGFR is indicative of improved organ perfusion.

Thus, the pharmaceutical formulations of the disclosure may, in a subject:
(a) reduce PVR;
(b) reduce mPAP;
(c) reduce ePAD;
(d) increase stroke volume (SV) of the heart;
(e) decrease systemic vascular resistance (SVR) and/or increase estimated glomerular filtration rate (eGFR);
(f) increase ejection fraction; and/or
(g) increase cardiac output;
as compared to baseline levels pre-administration. A combined decrease in SVR and increase in eGFR is indicative of improved organ perfusion. The change in one or more or all of these parameters may each result after 1-24 weeks of treatment. In some embodiments, the change in one or more or all of these parameters results after 24 weeks of treatment.

In particular embodiments, a reduction in mPAP as described herein may cause an improvement in dyspnea, as described in Solomonica A, et al. (2013) Circ Heart Fail. 6:53-60.

The pharmaceutical formulations of the disclosure are suitable for parenteral administration to a subject or patient. In some embodiments the subject or patient is a mammal, in particular a human.

Wild-type human Relaxin-2 has a half-life of minutes in vivo. As a consequence, it has to be administered by continuous intravenous infusion in hospitalized patients and presents severe side effects including blood pressure drop. In contrast, it will be understood that embodiments of the pharmaceutical formulations of the disclosure may be administered by injection, such as by intravenous, subcutaneous or intramuscular injection, to a subject or patient. In some embodiments, the pharmaceutical formulations are administered by subcutaneous injection. Administration by injection, such as by subcutaneous injection, offers the advantage of better comfort for the subject or patient and the opportunity to administer to a subject or patient outside of a hospital setting. In some embodiments the pharmaceutical formulation is administered by self-administration.

In some embodiments, the fusion polypeptides (thus including heterodimeric fusions) used in the formulation of the disclosure, e.g. HFUS1, have an increased half-life compared to wild-type Relaxin, which permits lower overall exposure based on molar concentration. For example, the fusion polypeptides (thus including heterodimeric fusions) used in the formulation of the disclosure, e.g. HFUS1, may be administered less frequently than wild-type Relaxin, thus providing a more convenient dosing schedule.

### Article of Manufacture

In one aspect, an article of manufacture is provided. In some embodiments, the article of manufacture includes a device or container that contains a pharmaceutical formulation that includes a heterodimeric fusion having Relaxin activity as defined herein, e.g. HFUS1, and a lipase-resistant surfactant as described herein. In an embodiment, the article of manufacture includes a device or container that contains a pharmaceutical formulation that includes a heterodimeric fusion having Relaxin activity as defined herein, e.g. HFUS1, and a lipase-resistant surfactant selected from poloxamer 188 (P188) and D-α-Tocopherol polyethylene glycol succinate (TPGS). In an embodiment, the article of manufacture includes a device or container that contains a pharmaceutical formulation that includes a heterodimeric fusion having Relaxin activity as defined herein, e.g. HFUS1, and poloxamer 188 (P188). In some embodiments, the container or device is a syringe, for example, a pre-filled syringe; an auto injector; bottle; vial; or test tube. In particular embodiments, the container or device is a syringe, optionally a pre-filled syringe. Accordingly, in particular embodiments, the article of manufacture includes a syringe, e.g. a pre-filled syringe, that contains a pharmaceutical formulation that includes a heterodimeric fusion having Relaxin activity as defined herein, e.g. HFUS1, and poloxamer 188 (P188). In some embodiments, the article of manufacture includes a device or container that contains the pharmaceutical formulation and a label on, or associated with, the device or container that provides directions for use. In some embodiments, the article of manufacture further includes other materials desirable from a commercial or user standpoint, including buffers, diluents, filters, needles, syringes, or package inserts with instructions for use.

In one aspect, a kit comprising the pharmaceutical formulation of the disclosure is provided. The kit may comprise a package containing the pharmaceutical formulations of the disclosure and instructions. In some embodiments, the kit includes at least one device or container that contains a pharmaceutical formulation that includes a heterodimeric fusion having Relaxin activity as defined herein, e.g. HFUS1, and a lipase-resistant surfactant as described herein. In some embodiments, the at least one device or container is selected from a syringe, for example, a pre-filled syringe; an auto injector; bottle; vial; or test tube. In particular embodiments, the at least one device or container is a syringe, for example, a pre-filled syringe.

The present disclosure provides a kit comprising the pharmaceutical formulations of the disclosure. The kit may comprise a package containing the pharmaceutical formulations of the disclosure and instructions for administration of the formulation.

In some embodiments, the kit includes at least one device or container that contains the pharmaceutical formulation of the disclosure and an injection device. In some embodiments, the injection device is adapted for intravenous, intramuscular or subcutaneous administration. In an embodiment, the injection device is adapted for subcutaneous administration. In particular embodiments, the injection device is a syringe, e.g. a pre-filled syringe, that contains a pharmaceutical formulation that includes a heterodimeric fusion having Relaxin activity as defined herein, e.g. HFUS1, and a lipase-resistant surfactant as defined herein, e.g. poloxamer 188 (P188).

In some embodiments, the pharmaceutical formulations of the disclosure are formulated in single dose vials or a container closure system (e.g. pre-filled syringe). Optionally associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kit includes instructions.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

Other examples and variations are within the scope of the disclosure, as set out in the appended claims. All documents cited herein are each entirely incorporated by reference herein, including all data, tables, FIGS. and text presented in the cited documents.

### SEQUENCES

TABLE 3
Sequence Listing. The upper hinge region is in Italics, Relaxin A is underlined,
Relaxin B is double underlined, the FC region is bold.

| SEQ ID NO: | Construct | Amino acid sequence |
|---|---|---|
| 1 | Relaxin A | QLYSALANKCCHVGCTKRSLARFC |
| 2 | Relaxin B | SWMEEVIKLCGRELVRAQIAICGMSTWS |
| 3 | FcH01 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPG |
| 4 | FcK01 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPG |
| 5 | Con01 | GGGGSGGGGSGGGGSGGGGGS |
| 6 | Con02 | PAPAPAPAPAPAPAPAPAPAG |
| 7 | RELAX0009 | SWMEEVIKLCGRELVRAQIAICGMSTWSGGGGSGGGGSG |
| | | GGGSQLYSALANKCCHVGCTKRSLARFCAAAGGGGSGG |
| | | GGSGGGGSGGGGSACPPCPAPEFEGGPSVFLFPPKPKD |
| | | TLMISRTPEVTCVVVDVSHEDPEVKFNWYVDGVEVHNAK |
| | | TKPREEQYNSTYRVVSVLTVLHQDWLNGKEYKCKVSNK |
| | | ALPASIEKTISKAKGQPREPQVYTLPPSREEMTKNQVSLT |
| | | CLVKGFYPSDIAVEWESNGQPENNYKTTPPVLDSDGSFF |
| | | LYSKLTVDKSRWQQGNVFSCSVMHEALHNHYTQKSLSL |
| | | SPG |
| 8 | RELAX0010 | DKTHTCPPCPAPELLGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPAPIEKTISK |
| | | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGGSGG |
| | | GGSGGGGSQLYSALANKCCHVGCTKRSLARFCGGGGSG |
| | | GGGSGGGGSSWMEEVIKLCGRELVRAQIAICGMSTWS |
| 9 | Rlx011 | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGGG |
| | | SGGGGSGGGGSGGGGGSQLYSALANKCCHVGCTKRSLA |
| | | RFC |
| 10 | Rlx011b | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPSREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGGG |
| | | SGGGGSGGGGSGGGGGSQLYSALANKCCHVGCTKRSLA |
| | | RFC |
| 11 | Rlx011DD | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLARFC |
| 12 | Rlx012 | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVCTLPPSREEMTKNQVSLSCAVKGFYP |
| | | SDIAVEWESNGQPENNYKTTPPVLDSDGSFFLVSKLTVD |
| | | KSRWQQGNVFSCSVMHEALHNHYTQKSLSLSPGGGGG |
| | | SGGGGSGGGGSGGGGGSQLYSALANKCCHVGCTKRSLA |
| | | RFC |
| 13 | Rlx012b | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGGGGGS |
| | | GGGGSGGGGSGGGGGSQLYSALANKCCHVGCTKRSLA |
| | | RFC |
| 14 | Rlx012DD | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGM |
| | | STWS |
| 15 | Rlx013 | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPCREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGGG |
| | | SGGGGSGGGGSGGGGGSSWMEEVIKLCGRELVRAQIAIC |
| | | GMSTWS |
| 16 | Rlx013b | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPSREEMTKNQVSLWCLVKGFYP |
| | | SDIAVEWESNGQPENNYKTTPPVLDSDGSFFLYSKLTVD |
| | | KSRWQQGNVFSCSVMHEALHNHYTQKSLSLSPGGGGG |
| | | SGGGGSGGGGSGGGGGSSWMEEVIKLCGRELVRAQIAIC |
| | | GMSTWS |
| 17 | Rlx013DD | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGSGGGGGSQLYSALANKCCHVGCTKRSLARFC |
| 18 | Rlx014 | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVCTLPPSREEMTKNQVSLSCAVKGFYP |
| | | SDIAVEWESNGQPENNYKTTPPVLDSDGSFFLVSKLTVD |
| | | KSRWQQGNVFSCSVMHEALHNHYTQKSLSLSPGGGGG |
| | | SGGGGSGGGGSGGGGGSSWMEEVIKLCGRELVRAQIAIC |
| | | GMSTWS |
| 19 | Rlx014b | GGAGGACPPCPAPEFEGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALPASIEKTI |
| | | SKAKGQPREPQVYTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGGGGGS |
| | | GGGGSGGGGSGGGGGSSWMEEVIKLCGRELVRAQIAIC |
| | | GMSTWS |
| 20 | Rlx014DD | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGM |
| | | STWS |
| 21 | Rlx020 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGGSQLYSALANKCCHVGCTKRSLARFC |
| 22 | Rlx021 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGGSSWMEEVIKLCGRELVRAQIAICGMSTWS |
| 23 | Rlx022 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGGSQLYSALANKCCHVGCTKRSLARFC |
| 24 | Rlx023 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGGSSWMEEVIKLCGRELVRAQIAICGMSTWS |
| 25 | Rlx024 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGGSQ |
| | | LYSALANKCCHVGCTKRSLARFC |
| 26 | Rlx025 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGGS |
| | | SWMEEVIKLCGRELVRAQIAICGMSTWS |
| 27 | Rlx026 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGAPAPAPAP |
| | | APAPAPAPAPAGSQLYSALANKCCHVGCTKRSLARFC |
| 28 | Rlx027 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGAPAPAPAP |
| | | APAPAPAPAPAGSSWMEEVIKLCGRELVRAQIAICGMSTWS |
| 29 | Rlx028 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGAAPAPAPA |
| | | PAPAPAGSQLYSALANKCCHVGCTKRSLARFC |
| 30 | Rlx029 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGAAPAPAPA |
| | | PAPAPAGSSWMEEVIKLCGRELVRAQIAICGMSTWS |
| 31 | Rlx030 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGAPAPAPAP |
| | | AGSQLYSALANKCCHVGCTKRSLARFC |
| 32 | Rlx031 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGAPAPAPAP |
| | | AGSSWMEEVIKLCGRELVRAQIAICGMSTWS |
| 33 | Rlx041E | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANECCHVGCTKRSLA |
| | | RFC |
| 34 | Rlx041H | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANHCCHVGCTKRSLA |
| | | RFC |
| 35 | Rlx041L | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANLCCHVGCTKRSLAR |
| | | FC |
| 36 | Rlx041M | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANMCCHVGCTKRSLA |
| | | RFC |
| 37 | Rlx044E | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANKCCHVGCTKESLAR |
| | | FC |
| 38 | Rlx044H | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANKCCHVGCTKHSLA |
| | | RFC |
| 39 | Rlx051A | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAAFC |
| 40 | Rlx0511 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAIFC |
| 41 | Rlx051M | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAMFC |
| 42 | Rlx051Q | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAQFC |
| 43 | Rlx051S | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLASFC |
| 44 | Rlx052E | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAREC |
| 45 | Rlx0521 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLARIC |
| 46 | Rlx055 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGM |
| | | STWSGGGSGGGSGQLYSALANKCCHVGCTKRSLARFC |
| 47 | Rlx056 | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGM |
| | | STWSGGGSGGGSGQLYSALANKCCHVGCTKRSLARFC |
| 48 | Rlx061H | SWMEEVIKLCGRELVRAQIAICGMSTWSAAAGGGGSGGG |
| | | GSGGGGSGGGGSACPPCPAPEFEGGPSVFLFPPKPKDT |
| | | LMISRTPEVTCVVVDVSHEDPEVKFNWYVDGVEVHNAKT |
| | | KPREEQYNSTYRVVSVLTVLHQDWLNGKEYKCKVSNKA |
| | | LPASIEKTISKAKGQPREPQVCTLPPSREEMTKNQVSLSC |
| | | AVKGFYPSDIAVEWESNGQPENNYKTTPPVLDSDGSFFL |
| | | VSKLTVDKSRWQQGNVFSCSVMHEALHNHYTQKSLSLS |
| | | PG |
| 49 | Rlx062K | QLYSALANKCCHVGCTKRSLARFCAAAGGGGSGGGGSG |
| | | GGGSGGGGSACPPCPAPEFEGGPSVFLFPPKPKDTLMIS |
| | | RTPEVTCVVVDVSHEDPEVKFNWYVDGVEVHNAKTKPRE |
| | | EQYNSTYRVVSVLTVLHQDWLNGKEYKCKVSNKALPASI |
| | | EKTISKAKGQPREPQVYTLPPCREEMTKNQVSLWCLVKG |
| | | FYPSDIAVEWESNGQPENNYKTTPPVLDSDGSFFLYSKLT |
| | | VDKSRWQQGNVFSCSVMHEALHNHYTQKSLSLSPG |
| 50 | Rlx076 | QLYSALANKCCHVGCTKRSLARFCAAAGGGGSGGGGSG |
| | | GGGSGGGGSACPPCPAPEFEGGPSVFLFPPKPKDTLMIS |
| | | RTPEVTCVVVDVSHEDPEVKFNWYVDGVEVHNAKTKPRE |
| | | EQYNSTYRVVSVLTVLHQDWLNGKEYKCKVSNKALPASI |
| | | EKTISKAKGQPREPQVYTLPPCREEMTKNQVSLWCLVKG |
| | | FYPSDIAVEWESNGQPENNYKTTPPVLDSDGSFFLYSKLT |
| | | VDKSRWQQGNVFSCSVMHEALHNHYTQKSLSLSPGGGG |
| | | GSGGGGSGGGGSGGGGGSQLYSALANKCCHVGCTKRS |
| | | LARFC |
| 51 | Rlx077 | SWMEEVIKLCGRELVRAQIAICGMSTWSAAAGGGGSGGG |
| | | GSGGGGSGGGGSACPPCPAPEFEGGPSVFLFPPKPKDT |
| | | LMISRTPEVTCVVVDVSHEDPEVKFNWYVDGVEVHNAKT |
| | | KPREEQYNSTYRVVSVLTVLHQDWLNGKEYKCKVSNKA |
| | | LPASIEKTISKAKGQPREPQVCTLPPSREEMTKNQVSLSC |
| | | AVKGFYPSDIAVEWESNGQPENNYKTTPPVLDSDGSFFL |
| | | VSKLTVDKSRWQQGNVFSCSVMHEALHNHYTQKSLSLS |
| | | PGGGGGSGGGGSGGGGSGGGGGSSWMEEVIKLCGREL |
| | | VRAQIAICGMSTWS |
| 52 | Rlx014DDdel2aa | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGM |
| | | ST |
| 53 | Rlx014DDdel3aa | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVCTLPPSREEMTKNQVSLSCAVKGFYPSDI |
| | | AVEWESNGQPENNYKTTPPVLDSDGSFFLVSKLTVDKSR |
| | | WQQGNVFSCSVMHEALHNHYTQKSLSLSPGGGGGSGG |
| | | GGSGGGGSGGGGGSSWMEEVIKLCGRELVRAQIAICGMS |
| 54 | R347L | ELVLTQPASVSGSPGQSITISCTGTSSDVGGYNYVSWYQQ |
| | | HPGKAPKLMIYDVSKRPSGVSNRFSGSKSGNTASLTISGQ |
| | | AEDEADYYCSSYTSSSTLVFGGGTKLTVLGQPKAAPSVTL |
| | | FPPSSEELQANKATLVCLISDFYPGAVTVAWKADSSPVAG |
| | | VETTTPSKQSNNKYAASSYLSLTPEQWKSHRSYSCQTHE |
| | | GSTVEKTVAPTECS |
| 55 | R347Rlx011DD | EVQLLESGGGLVQPGGSLRLSCTTSGFTFNTYAMSWVRQ |
| | | APGKGLEWLSGINNNGRTAFYADSVKGRFTISRDNSKNTL |
| | | YLQINSLRADDTAVYFCAKDVRFIAVPGDSWGQGTLVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPEFEG |
| | | GPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPASIEKTISKAKGQPREPQVYTLPPC |
| | | REEMTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSPGGGGGSGGGGSGGGGSGGGGGSQ |
| | | LYSALANKCCHVGCTKRSLARFC |
| 56 | R347Rx014DD | EVQLLESGGGLVQPGGSLRLSCTTSGFTFNTYAMSWVRQ |
| | | APGKGLEWLSGINNNGRTAFYADSVKGRFTISRDNSKNTL |
| | | YLQINSLRADDTAVYFCAKDVRFIAVPGDSWGQGTLVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPEFEG |
| | | GPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPASIEKTISKAKGQPREPQVCTLPPS |
| | | REEMTKNQVSLSCAVKGFYPSDIAVEWESNGQPENNYKT |
| | | TPPVLDSDGSFFLVSKLTVDKSRWQQGNVFSCSVMHEAL |
| | | HNHYTQKSLSLSPGGGGGSGGGGSGGGGSGGGGGS |
| | | SWMEEVIKLCGRELVRAQIAICGMSTWS |
| 57 | RELAX0126 | DKTHTCPPCPAPELLGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPAPIEKTISK |
| | | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGSPQLYS |
| | | ALANKCCHVGCTKRSLARFCGGGSGGGSGSWMEEVIKL |
| | | CGRELVRAQIAICGMSTWS |
| 58 | RELAX0127 | DKTHTCPPCPAPELLGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPAPIEKTISK |
| | | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGSGGSPQ |
| | | LYSALANKCCHVGCTKRSLARFCGGGSGGGSGSWMEEVI |
| | | KLCGRELVRAQIAICGMSTWS |
| 59 | RELAX0128 | DKTHTCPPCPAPELLGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPAPIEKTISK |
| | | AKGQPREPQVYTLPPSRDELTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGSGGSGG |
| | | SPQLYSALANKCCHVGCTKRSLARFCGGGSGGGSGSWM |
| | | EEVIKLCGRELVRAQIAICGMSTWS |
| 60 | Linker 01 | GGGSGGGSGG |
| 61 | Rlx052A | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLARAC |
| 62 | RELAX0013 B | DSWMEEVIKLCGRELVRAQIAICGMSTWS |
| | chain | |
| 63 | RELAX0013 A | QLYSALANKCCHVGCTKRSLARFC |
| | chain | |
| 64 | RELAX0014 B | MRVSEEWMDGFIRMCGREYARELIKICGASVGR |
| | chain | |
| 65 | RELAX0014 A | ESGGLMSQQCCHVGCSRRSIAKLYC |
| | chain | |
| 66 | Rlx042R | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPCREEMTKNQVSLWCLVKGFYPS |
| | | DIAVEWESNGQPENNYKTTPPVLDSDGSFFLYSKLTVDK |
| | | SRWQQGNVFSCSVMHEALHNHYTQKSLSLSPGGGGGS |
| | | GGGGSGGGGSGGGGGSQLYSALANKCCRVGCTKRSLA |
| | | RFC |
| 67 | Rlx014d | DKTHTACPPCPAPEFEGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVCTLPPSREEMTKNQVSLSCAVKGFYPS |
| | | DIAVEWESNGQPENNYKTTPPVLDSDGSFFLVSKLTVDK |
| | | SRWQQGNVFSCSVMHEALHNHYTQKSLSLSPGGGGGS |
| | | GGGGSGGGGSGGGGGSSWMEEVIKLCGRELVRAQIAIC |
| | | GMSTWS |
| 68 | Rlx051Y | DKTHTCPPCPAPEFEGGPSVFLFPPKPKDTLMISRTPEVT |
| | | CVVVDVSHEDPEVKFNWYVDGVEVHNAKTKPREEQYNS |
| | | TYRVVSVLTVLHQDWLNGKEYKCKVSNKALPASIEKTISK |
| | | AKGQPREPQVYTLPPCREEMTKNQVSLWCLVKGFYPSDI |
| | | AVEWESNGQPENNYKTTPPVLDSDGSFFLYSKLTVDKSR |
| | | WQQGNVFSCSVMHEALHNHYTQKSLSLSPGGGGGSGG |
| | | GGSGGGGSGGGGGSQLYSALANKCCHVGCTKRSLAYFC |

### EXAMPLES

### Materials and Methods

### Materials

All the materials used for the study were of USP or multi-compendial grade. All the solutions and buffers were prepared using USP or HPLC water and were filtered through 0.2 μm PVDF filters (Millipore, Millex GV, SLG033RB) before further use. HFUS1 samples for formulation screening and stability studies were prepared under sterile aseptic conditions in a Biosafety Cabinet (BSC). Bulk material was stored at 5° C. All formulations were filtered through 0.22 μm PVDF filter units and filled into 2R glass vials or pre-filled syringes (PFS) and stored in incubators set at 5, 25 and 40° C. for stability timepoints.

### Methods

### Protein Concentration Determination

HFUS1 protein concentrations were determined by measuring absorbance at 280 nm with Trinean HT-A280 using a procedure adapted from SOP DV-050465. A measured extinction coefficient of 1.47 (mg/mL) 1 cm 1 was used to calculate protein concentrations.

### Purity Determination by Size Exclusion Chromatography

Size exclusion chromatography was conducted in Agilent Technologies 1200/1100 HPLC system with temperature controlled autosampler at 8° C., using a TSKgel G3000SWXL, 5 μm, 250 Å, column (7.8×300 mm) and Guard Column for 7.8 mm TSKgel columns from Tosoh Bioscience and Agilent ChemStation software. The UV lamp was turned on 30 minutes before analysis and the system was primed with mobile phase (0.1 M Sodium Phosphate Dibasic Anhydrous, 0.1 M Sodium Sulphate pH 6.8) at 1.0 ml/min for at least 30 minutes monitoring the pressure to a maximum of 130 bar. Samples were diluted to 10 mg/mL in PBS and filtered using Ultrafree-MC Centrifugal filters with PVDF membrane and 0.45 μm pore size (Merck). All samples were run at 280 nm with a column flow of 1.0 ml/min and a stop time of 20 min, injection volume of 25 μl and column temperature of 20° C.

### Isoelectric Point Determination

Capillary isoelectric focusing (cIEF) was used to determine the isoelectric point of HFUS1 using ProteinSimple's Maurice, per the manufacturer's protocols (Bio-Techne®).

### Visual Appearance Assessment

Before performing the visual testing of formulations, all samples and standards were allowed to equilibrate at room temperature. The outside of the glass vials was wiped with alcohol to remove any particle or residue on the glass surface. Visual inspection was performed manually in a white and black background light cabinet (Apollo II Liquid Viewer, Adelphi Manufacturing Co Ltd.) with a Lux range of 3000-3400. Cabinet lights were turned on 20 minutes before use to ensure appropriate light intensity.

Determination of visible particles was performed by comparing samples to particle standards with increasing concentrations of Barium Sulphate. Particles standards are numbered 0 to 7, with 7 being the highest number of particles, and served as a guideline when determining visible particles in samples. Samples were swirled 10-20 times to ensure homogeneity and were compared to standards against the black background at approximately halfway up from the panel and directly below the light at the top of the panel.

### Sub-Visible Particle Analysis

Sub-visible particle counting was performed as indicated in the text using either MFI or HIAC.

For MFI, a MFI 5200 series model was used with a 100 μm 1.6 mm silane coated flow cell (Protein simple, United States). Before use, the system was cleaned with approximately 10 mL of non-ionized ultra-pure water followed by 1 mL of 10% Decon and rinsed with 10 mL non-ionized ultrapure water using the System Flush Preview mode. The cell was allowed to dry to check if the silane coating was intact and ensure that no particles were attached to its surface. If the cell was dirty the cleaning procedure was performed again. To assess the system cleanliness a water background run was performed by priming the cell with 250 μl of water at 1 ml/min and analysing approximately 800 μl of the water sample with optimized illumination. The live images were monitored to check for large particles or air bubbles entering the system.

For HIAC, a High Accuracy Products (HIAC) system model 9703 equipped with a HRLD150 sensor was used. Samples were diluted to 25 mg/mL before analysis and degassed for 30 min before measurements were taken. For each sample, the particle counts of the representative buffer were acquired to assess the contribution of the dilution buffer to total particle counts. Four measurements were taken for each sample.

### Differential Scanning Calorimetry (DSC) Measurement of Unfolding Temperature

DSC experiments were performed in an automated MicroCal VP-Capillary DSC system from Malvern Panalytical (United Kingdom). Samples were diluted to 5 mg/ml using the corresponding formulation buffer and filtered with 0.22 μm PVD centrifugal filter units. Lysozyme (3 mg/ml) was used as a standard at the beginning and end of every run to assess the system suitability. The scans were run from 25° C. to 100° C. at a scan rate of 95° C./hr with a filtering period of 24 s and cleaning cycle between protein sample sets. For each sample the matching formulation buffer was used as reference and analysed under identical conditions. Data analysis was performed with Origin v 7.0552 software. Buffer blank scans were subtracted from the thermograms and normalized to the appropriate protein concentration. Data was fitted using the Non-2-state model with cursor initialization of each transition to determine the thermodynamic parameters.

### Dynamic Light Scattering (DLS) Measurement

DLS measurements were made using a Wyatt DynaPro PlateReader II (Wyatt, Santa Barbara, CA) with a laser wavelength of 820.17 nm. Three independent samples were prepared for each sample and 30 μl loaded into wells on a 384 well black non-treated polystyrene plate (Thermo Scientific Nunc, UK) over a protein concentration range of 4-20 mg/ml. For each well, ten DLS measurements of 5 s each were acquired at 20° C. and the data were discarded if the percent polydispersity was >15%. Cumulants analysis was performed using the Wyatt Dynamics Software to directly measure the protein self-diffusion coefficient (D) in the three samples for each condition, which were then averaged. Since D scales with protein concentration (c) according to the equation: D=D₀(1+k_{D}.C), where D₀ is the protein diffusion coefficient at infinite dilution, the protein-protein interaction parameter, k_{D}, can be determined from a plot of D vs c.

### Measurement of Amino Acid Clipping by Reversed-Phase High-Performance Liquid Chromatography (RP-HPLC)

Reverse Phase-High Performance Liquid Chromatography was used to measure major product peaks and b-chain clipping whereby separation is achieved by differential affinity with hydrophobic chains of the packing material in a column. Test sample concentration was adjusted and mixed with a denaturing buffer in the presence of a reducing agent and incubated at a designated temperature for a specific amount of time. The sample was then injected in a reverse phase column and eluted with a gradient of increasing organic solvent. Peaks eluted according to their hydrophobicity.

More specifically, an Agilent 1260 Infinity series or equivalent instrument was used for the Reverse Phase-High Performance Liquid Chromatography method. The column used was the Phenomenex, Aeris WIDEPORE 3.6 μm XB-C8 HPLC column, 4.6×150 mm. 100 μg of each sample was denatured (8M Guanidine, 130 mM Tris (hydroxymethyl)aminomethane, 1 mM EDTA pH 7.6) in the presence of a reducing agent (1M DTT), then mixed and incubated at 37° C. for 45 minutes±5 minutes. The final concentration of each sample was 0.5 mg/mL. For each sample, 10 μL was injected onto the column and eluted with a 40-minute gradient of increasing organic solvent (Mobile Phase A: 95% Water, 5% Acetonitrile, 0.1% TFA and Mobile Phase B: 80% Acetonitrile: 20% Water, 0.1% TFA). Peaks eluted according to their hydrophobicity, with detection carried out at 220 nm.

### Potency Assay to Assess the Binding of the HFUS1 to the RXFP1 Receptor

THP1 cells which endogenously express RXFP1 receptor were engineered to express CRE-NanoLuc upon RXFP1 receptor activation. In the assay, HFUS1 binds to RXFP1 receptors on the surface of THP1-CRE NanoLuc cells. This leads to the production of cyclic adenosine monophosphate (CAMP), which signals through CAMP response element (CRE) to drive expression of a Nanoluciferase enzyme. Expression of the luciferase enzyme is then measured by addition of a chemiluminescent substrate. EC50 values representing the concentration of HFUS1 at which half-maximal luciferase expression is observed are generated using a four parameter semilogistical curve fit for the HFUS1 Reference Standard (HFUS1 formulated in 20 mM Histidine/Histidine HCL, 190 mM arginine HCl, 0.02% (w/v) PS80 at pH 6.5) and test samples. The relative potency of each HFUS1 sample was assessed by dividing the EC50 value of the Reference Standard by the EC50 value of each sample and multiplying by 100%.

### Example 1: Preliminary Stability Assessment of HFUS1

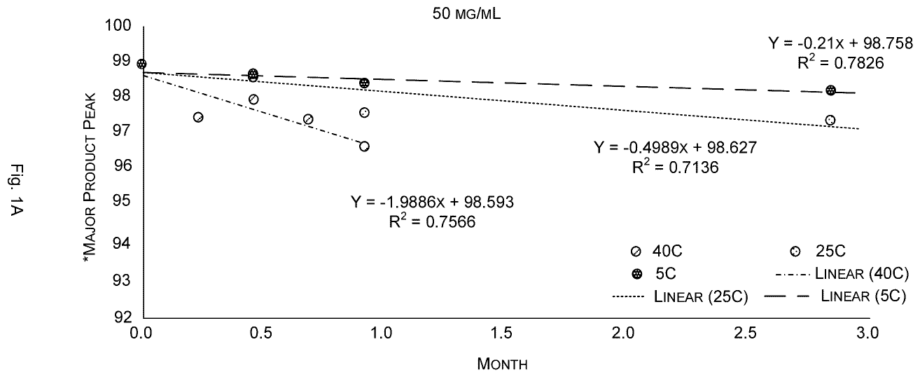

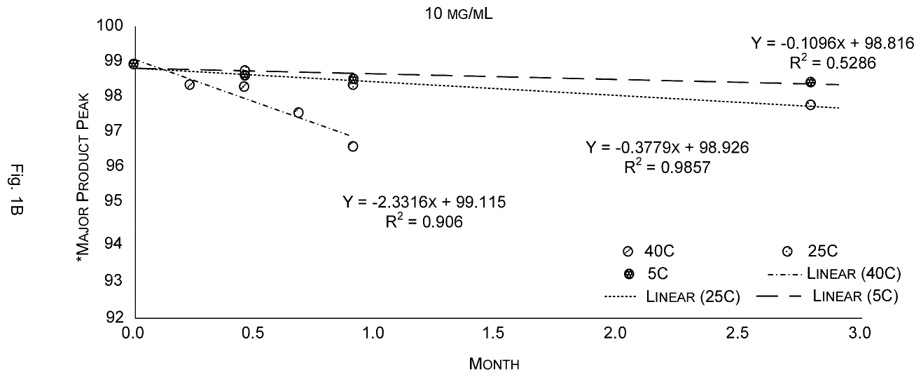

The liquid stability of HFUS1 was first evaluated in a histidine buffer-based formulation containing sucrose and polysorbate (PS) 80 at an HFUS1 concentration of 10 and 50 mg/mL. The purity loss of the molecule during storage at 5, 25, and 40° C. was measured by HPSEC and shown in FIG. 1. The major product peak (MMP) % rate losses at different temperatures are taken from the slope of the linear regression of the data points: a) 50 mg/mL, −0.2% for 5° C., −0.5% for 25° C. and −2.0% for 40° C.; and b) 10 mg/mL, −0.1% for 5° C., −0.4% for 25° C. and −2.3% for 40° C.

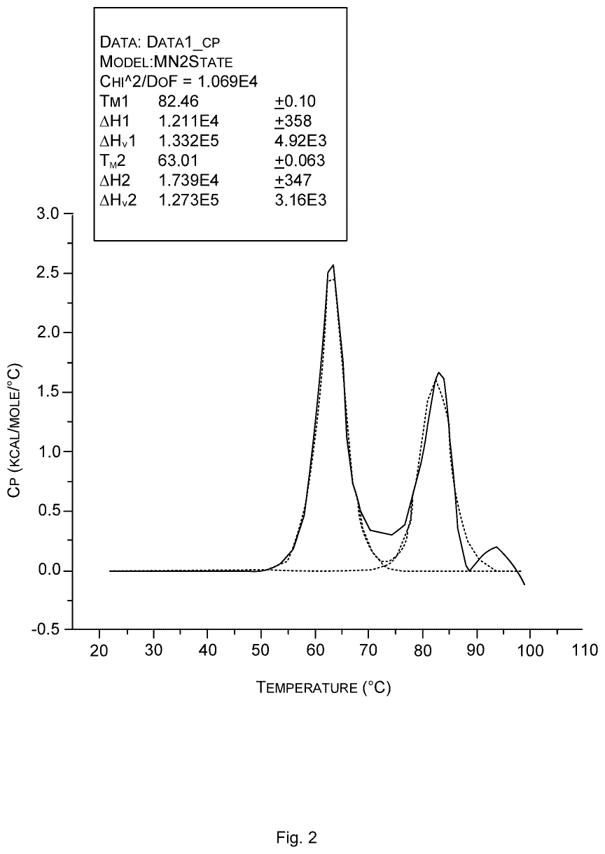

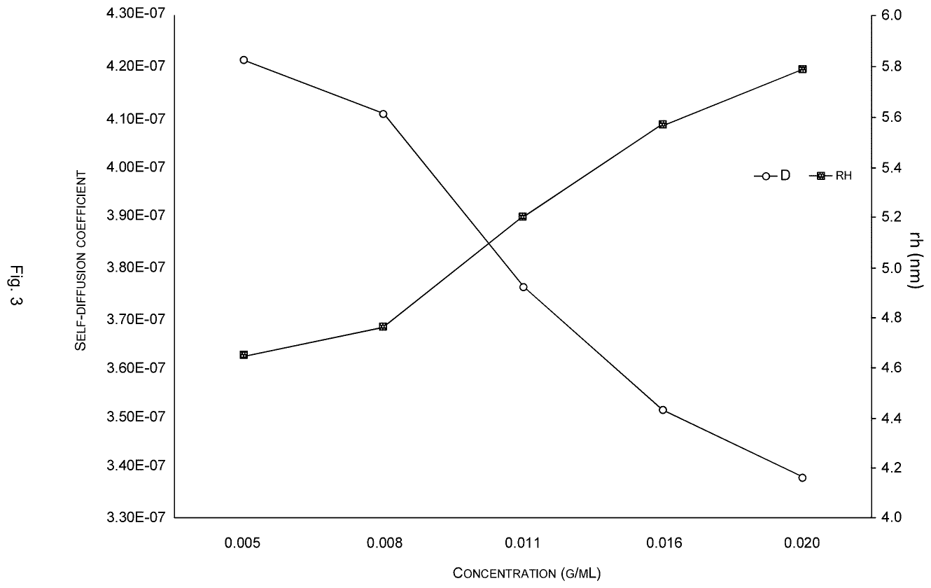

The conformational stability of the molecule was assessed by measuring the unfolding temperature (Tₘ) of the molecule in the histidine/sucrose formulation buffer. The DSC trace is shown in FIG. 2. HFUS1 has two unfolding events. The Tₘ onset was around 57° C. with the first unfolding is at 63° C., and the second unfolding was at 82° C.

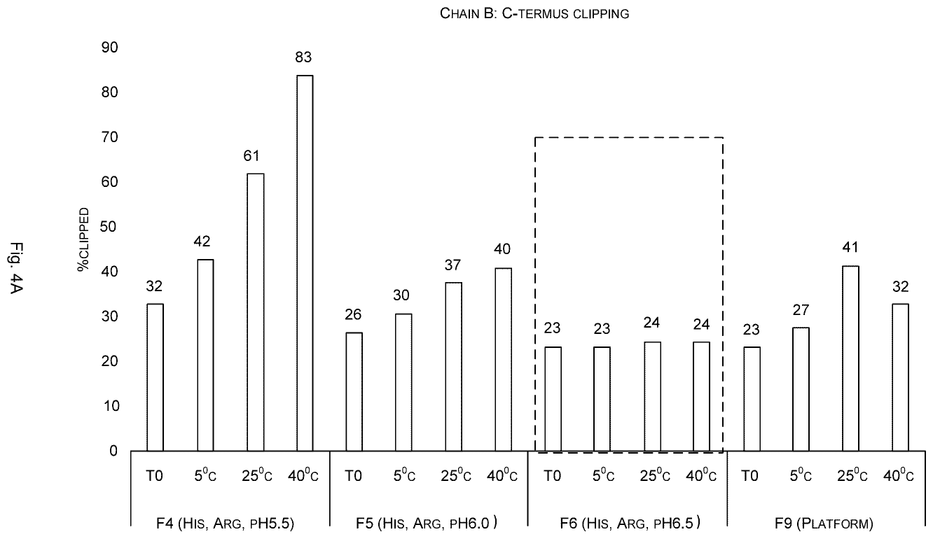

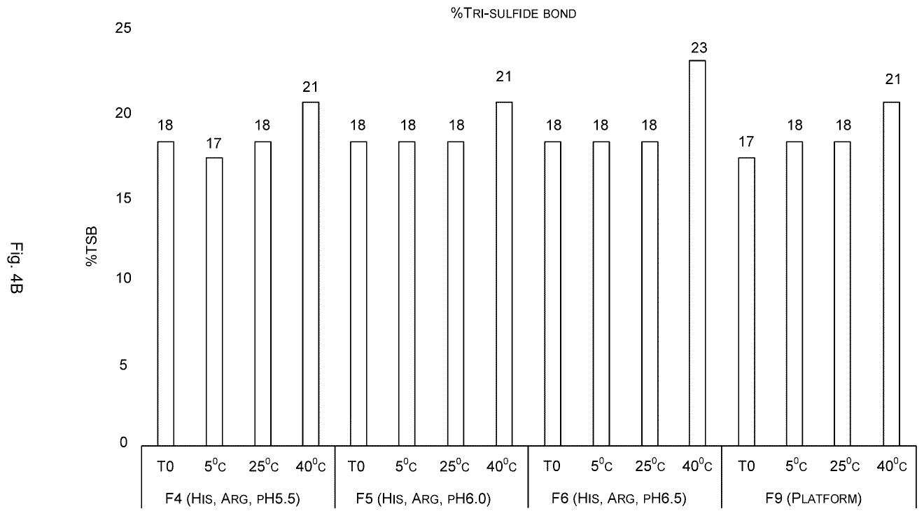

The protein-protein interaction parameter (k_{D}) of HFUS1 was evaluated by DLS using the method described in the method section. As a rule of thumb, a negative k_{D} value indicates a higher tendency of attractive force between HFUS1 molecules, while a positive value indicates a repulsive protein interaction. The self-diffusion coefficient and hydrodynamic radius of HFUS1 are shown in FIG. 3. The k_{D} of the molecule was calculated as −16 mL/g indicating a tendency of molecular self-association.

Meanwhile, no phase separation or changes in visual appearance were observed with HFUS1 on storage. In general, HFUS1 showed an acceptable stability profile during the assessment. However, despite low degradation rates at 25 and 40° C., from limited timepoint of data (up to 3 months), the molecule showed a relatively high aggregation rate by HPSEC at 5° C., i.e. −0.21% per month at 50 mg/mL. If this rate continues, it translates to around 2.5% aggregation or monomer loss per year, making the molecule challenging to meet shelf-life requirements. This high aggregation rate at 5° C. was likely due to the self-association tendency of the molecule in the formulation, as reflected by the negative k_{D}. If a molecule is prone to self-association, as the association continues, the molecules may form soluble aggregates (normally detectable by HPSEC) which can become precursors of insoluble, large-size aggregates, and particles eventually, significantly impacting the stability profile of the molecule. This data signalled that formulation optimisation was required to reduce this self-association and aggregation tendency.

### Example 2: Phase 1 Formulation Optimisation and Development

Optimisation of HFUS1 formulation was conducted. This was focused on pH and buffer species screening as well as the use of alternative excipient to sucrose as a stabilising agent. A pH range of 4.5 to 6.5 was evaluated and a citrate or histidine buffer system was used to cover this range. Meanwhile arginine HCl or lysine HCl was used to replace sucrose in the formulation. PS80 remained as the surfactant. HFUS1 was used at a concentration of 50 mg/mL. DLS was used to measure the interaction parameter k_{D}, DSC for onset of the unfolding temperature, HPSEC for purity and visual assessment for particle formation levels. A summary of the results is shown in Table 4. Results generated from the preliminary study for the histidine-sucrose formulation was included for comparison purpose. Additionally the histidine-sucrose formulation was also repeated in this study to serve as a control, considering that new HFUS1 material was used in this optimisation study.

TABLE 4
Summary of DLS, DSC, HPSEC and visual data of HFUS1 formulations.

| | | | | HPSEC 40 C. | | | HPSEC 2-8° C. | | | | | |
|---|---|---|---|---|---|---|---|---|---|---|---|---|
| | | | | rate/month | | | rate/month | | | | | |
| | Condition (all | DLS | DSC | (1 month) | | | (6 months) | | | Visual 5° C. | | |
| | with 0.02% | K_{D} | Tₒₙₛₑₜ | % | % | % | % | % | % | (6 months) | | |
| Formulation | PS80) | value | (° C.) | mon | agg | frag | mon | agg | frag | Particle | Opal. | Colour |
| 1 | pH 4.5 + Citrate + | −6.8^{a} | 39.9 ^{e} | Phase separated/ | | | −0.09 ⁱ | −0.02 ^{a} | 0.11 ⁱ | 1 ^{a} | <II ^{a} | Y4 ^{a} |
| | Arg HCl | | | precipitation ^{e} | | | | | | | | |
| 2 | pH 5.0 + Citrate + | −6.5^{a} | 45.3 ⁱ | −26.53 ^{e} | 19.58 ^{e} | 6.95 ^{e} | −0.04 ^{a} | −0.01 ^{a} | 0.07 ^{a} | 3 ⁱ | <II ^{a} | Y4 ^{a} |
| | Arg HCl | | | | | | | | | | | |
| 3 | pH 5.5 + | −11.5 ⁱ | 48.1 ⁱ | −6.03 | 3.19 ⁱ | 2.84 ⁱ | −0.01 ^{a} | 0.01 ^{a} | 0 ^{a} | 4 ^{e} | <II ^{a} | Y4 ^{a} |
| | Citrate + Arg | | | | | | | | | | | |
| | HCl | | | | | | | | | | | |
| 4 | pH 5.5 + | −5.3 ^{a} | 46.7 ⁱ | −7.27 ^{e} | 3.62 ⁱ | 3.64 ⁱ | −0.03 ^{a} | 0.03 ^{a} | 0 ^{a} | 2 ^{a} | II ⁱ | Y4 ^{a} |
| | Histidine + Arg | | | | | | | | | | | |
| | HCl | | | | | | | | | | | |
| 5 | pH 6.0 + | −9.3 ⁱ | 50.1 ⁱ | −4.17 ^{a} | 2.9 ^{a} | 1.26 ^{a} | −0.05 ^{a} | 0.05 ^{a} | 0 ^{a} | 1 ^{a} | <II ^{a} | Y4 ^{a} |
| | Histidine + Arg | | | | | | | | | | | |
| | HCl | | | | | | | | | | | |
| 6* | pH 6.5 + | −6.2 ^{a} | 50.9 ⁱ | −2.9 ^{a} | 2.9 ^{a} | 0 ^{a} | −0.08 ^{a} | 0.08 ^{a} | 0 ^{a} | 1 ^{a} | <II ^{a} | Y4 ^{a} |
| | Histidine + Arg | | | | | | | | | | | |
| | HCl | | | | | | | | | | | |
| 7 | pH 5.5 + Citrate + | −12.6 ⁱ | 50.5 ⁱ | −5.85 ⁱ | 3.99 ⁱ | 1.86 ⁱ | −0.04 ^{a} | 0.04 ^{a} | 0 ^{a} | <3 ⁱ | <II ^{a} | Y4 ^{a} |
| | Sucrose | | | | | | | | | | | |
| 8 | pH 5.5 + | −10.9 ⁱ | 50.4 ⁱ | −4.67 ^{a} | 2.56 ^{a} | 2.11 ⁱ | −0.02 ^{a} | 0.02 ^{a} | 0 ^{a} | <4 ^{e} | II ⁱ | Y4 ^{a} |
| | Citrate + Lys HCl | | | | | | | | | | | |
| 9 Control | pH 6.0 + | −15.2 ^{e} | 52.1 ⁱ | −5.82 ^{e} | 4.81 ⁱ | 1.01 ^{a} | −0.10 ⁱ | 0.10 ⁱ | 0 ^{a} | 3 ⁱ | II ⁱ | Y4 ^{a} |
| | Histidine + | | | | | | | | | | | |
| | Sucrose | | | | | | | | | | | |
| Developability | pH 6.0 + | −16 ^{e} | 56.9 ^{e} | −1.99 ^{a} | 1.99 ^{a} | 0 ^{a} | −0.07 | 0.07 | 0.00 | — | — | — |
| | Histidine + | | | | | | | | | | | |
| | Sucrose | | | | | | | | | | | |

Abbreviation: mon—monomer, agg—aggregation, frag—fragmentation, and opal—opalescence
^{a} acceptably within target threshold;
ⁱ near max tolerated threshold;
^{e} exceeds tolerated threshold

In the repeated study (histidine-sucrose formulation, control), HFUS1 showed similar Ko as the one measured in the developability study. However, it has a lower Tₒₙₛₑₜ and higher aggregation at 40° C. This difference is likely caused by the variability of the material used for this study. In the developability study, material from transient expression was used while in the optimisation study, Chinese Hamster Ovary (CHO) cells were used. This indicates that process variability is likely to impact the product quality of the molecule and a robust formulation is needed to meet the stability requirement.

As shown in Table 4, at the low pH end (pH 4.5 or pH 5.0), significant purity loss was seen by HPSEC after storage at the stressed condition (40° C.) for 1 month. Formulation 2 showed high level of aggregation and fragmentation while formulation 1 resulted in severe phase separation and precipitation. The conformational stability of the molecule is also lower at lower pHs, since lower unfolding onset temperatures were seen for formulations 1, 2, 3 and 4. Buffer species and excipients appear to have an impact to the stability of the molecule. Several variables were investigated at the same pH. At pH 5.5, there are formulations containing citrate or histidine buffer system, and also formulations containing Arginine HCl, Lysine HCl, and sucrose. Sucrose and Lysine HCl appeared to offer slightly higher Tₒₙₛₑₜ, which means a better conformational stability, while histidine system offered better colloidal stability to the molecule represented by a higher K_{D} value: i.e. formulation 4 (histidine) has a higher Ko than formulation 3 (citrate). Arginine HCl did not show improved K_{D} over sucrose at pH 5.5, for example formulation 3 (arginine HCl) has a K_{D} of −11.5 which is similar to −12.6 of formulation 7 (sucrose). However, at pH 6.0, arginine HCl (formulation 5) showed some improvement compared to sucrose (formulation 9), with a higher K_{D} and lower aggregation after 40° C. storage. Overall, formulation 6 shows the best stability profile with the highest Ko, and lowest purity loss. This is likely a combined effect of pH, buffer system and the use of arginine HCl.

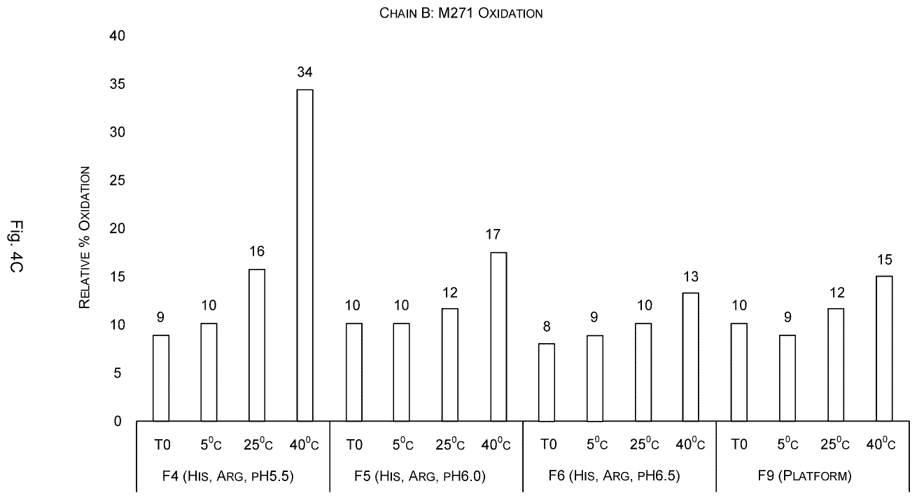

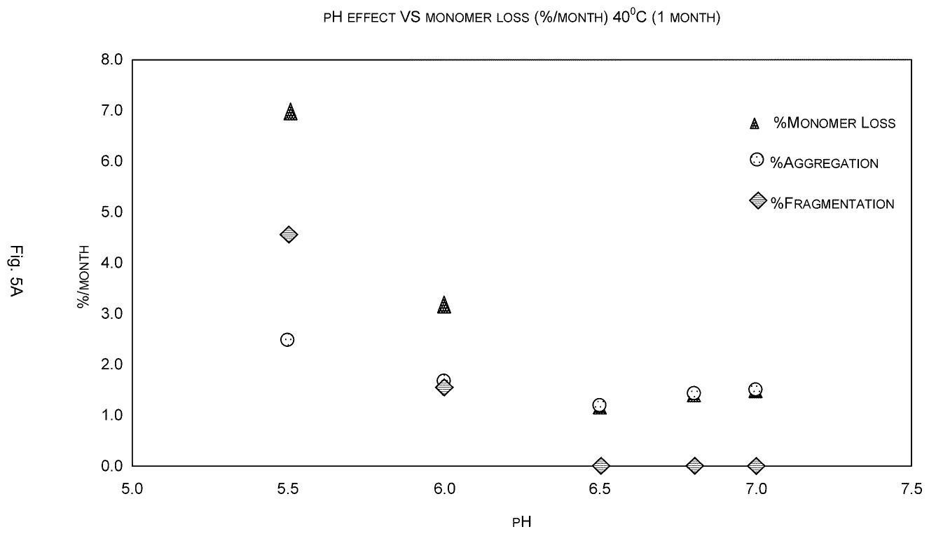

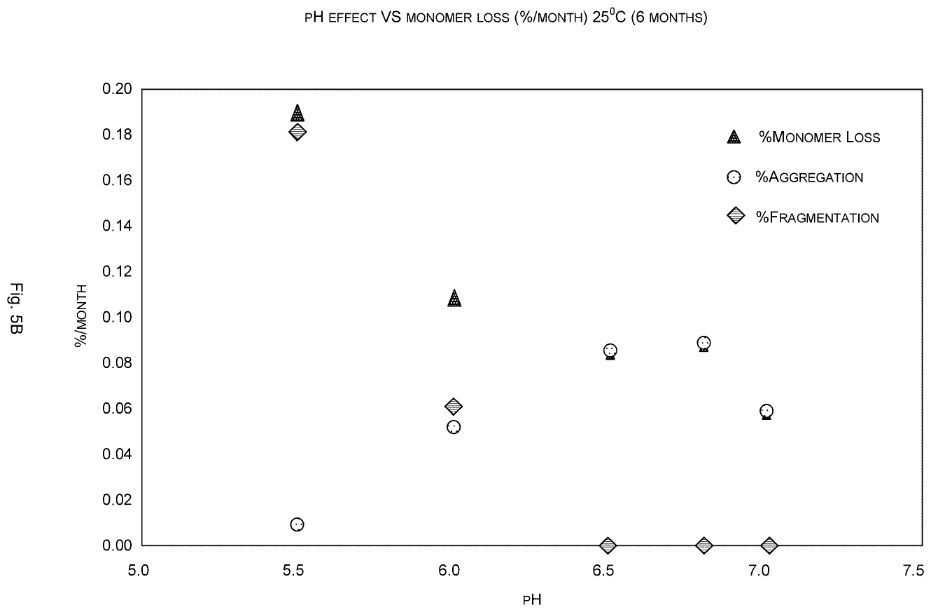

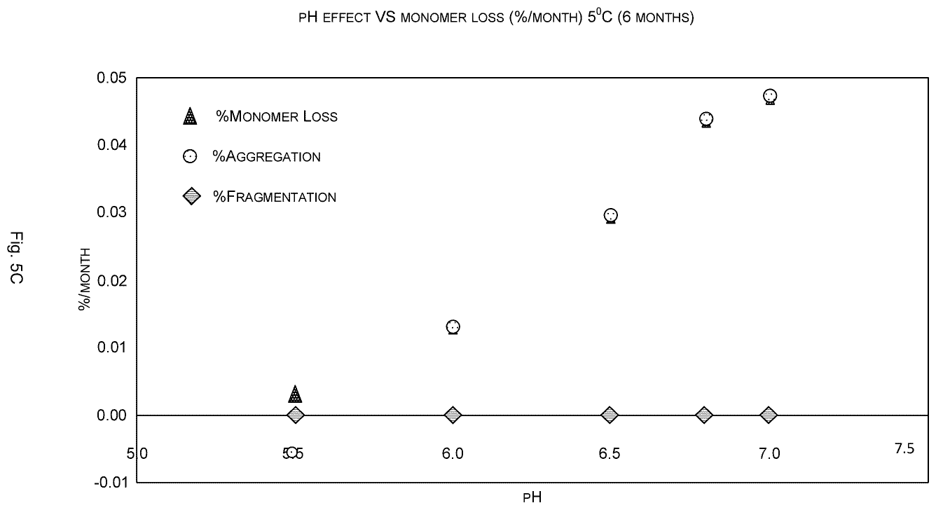

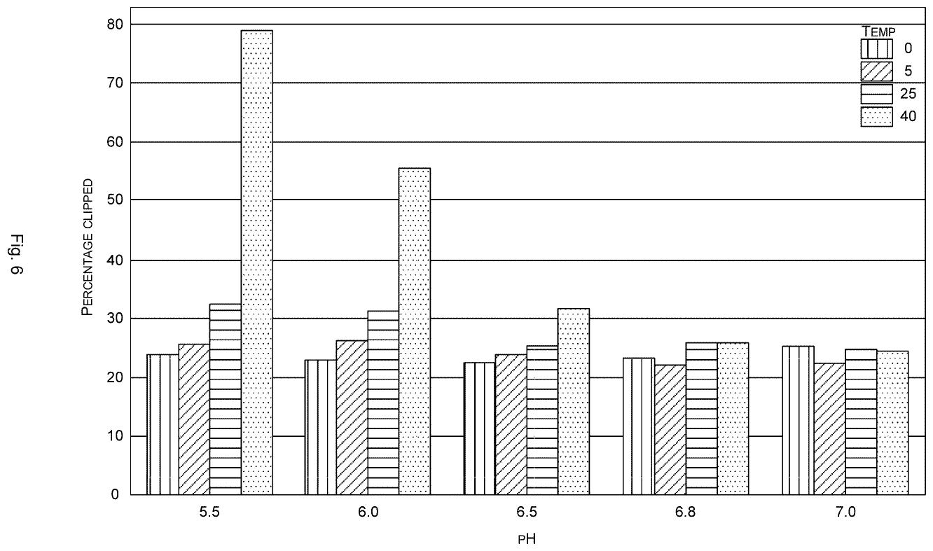

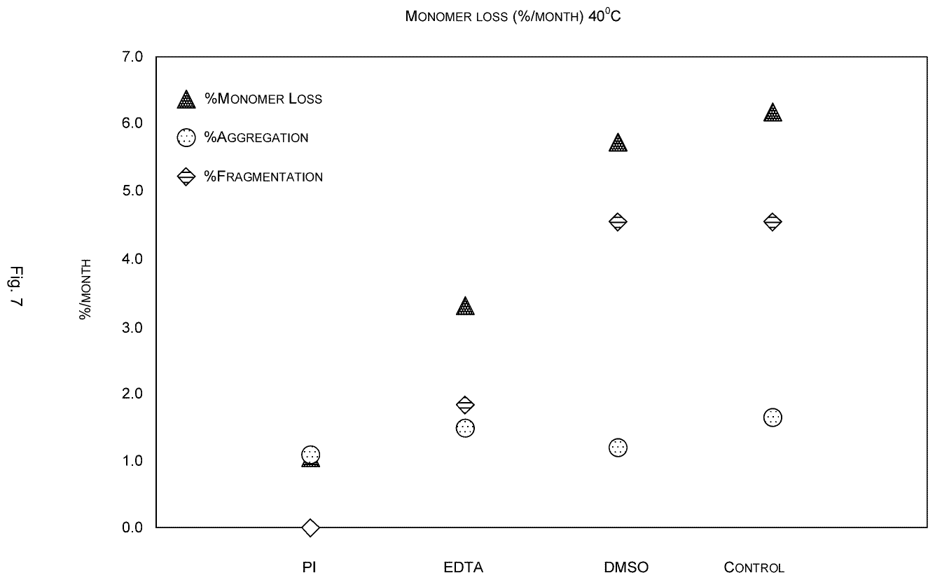

Chemical stability of the molecule in the formulations were further assessed by mass spectrometry (MS), with a focus on the histidine-arginine HCl formulation system, to evaluate impact of pH on the molecule. Histidine-sucrose formulation was included as a control for comparison. The attributes assessed were: 1) clipping of amino-acid (AA) at the C-terminus of chain B of relaxin; 2) change of % tri-sulphide bond; and 3) Methionine 271 (M271) oxidation at chain B of relaxin. The results are summarised in FIG. 4.

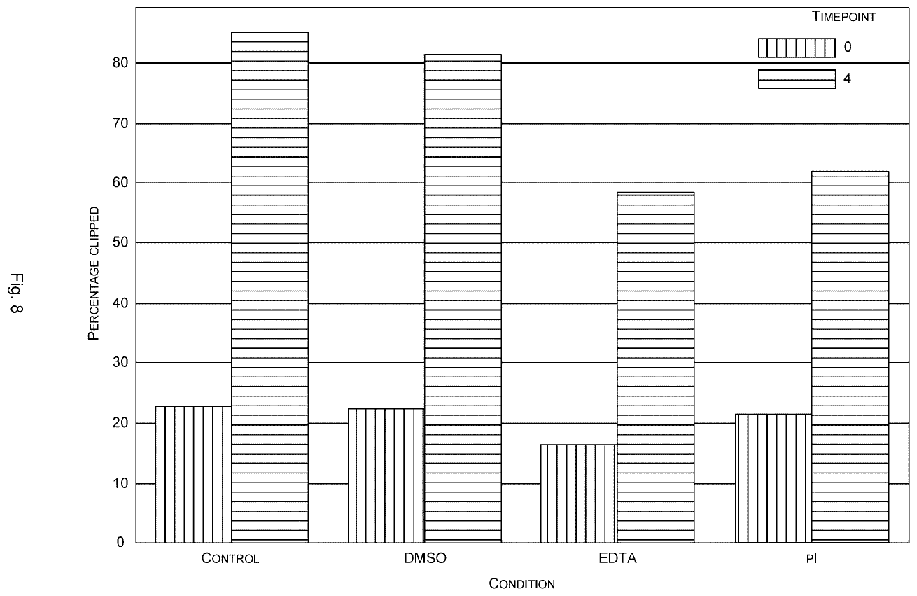

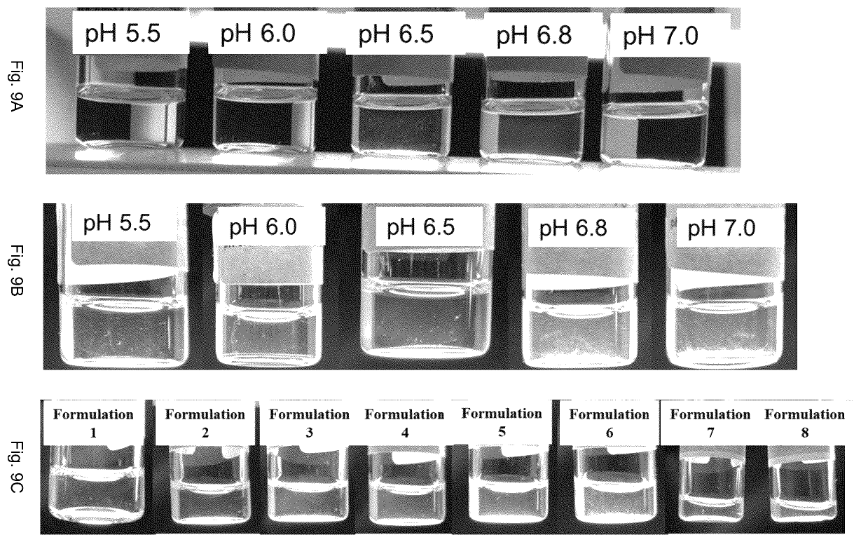

No apparent change in % tri-sulphide bond was seen across the pH tested. However, pH 5.5 showed the highest clipping compared to pH 6.0 and pH 6.5 with the latter appearing the best. pH 5.5 also showed higher oxidation compared to higher pH, though the difference was less obvious compared to clipping. To further evaluate the histidine-arginine HCl formulation system, pH optimisation study was conducted. Histidine-arginine HCl formulation formulations were prepared at a pH of 5.5, 6.0, 6.5, 6.8 and 7.0. The focus was placed on HPSEC on HFUS1 purity loss and data is shown in FIG. 5.

Fragmentation of HFUS1 appeared to be high at low pH. At both 25 and 40° C., pH 5.5 showed the highest fragmentation of the molecule, followed by pH 6.0. At both temperatures, fragmentation dropped to 0 from pH 6.5 and onwards. No fragmentation was seen at 5° C. at all pH tested. As for aggregation, different profiles were observed at different temperatures. At 40° C., highest aggregation was seen at pH 5.5. As the pH increased, a V-shape was observed with pH 6.5 showing the slowest aggregation rate, while at 5° C., as the pH increased, the aggregate rate also increased, albeit all at a low level, a clear trend was seen. 5° C. condition is most representative to real time storage of the drug product and in the histidine-arginine HCl system, all formulations offer improved stability compared to histidine-sucrose. Even at pH 7.0 with the highest aggregation rate, a 0.05% monthly rate represents around 0.6% drop per year. The clipping of the molecule in this range of pH was also assessed by mass spectroscopy with data summarised in FIG. 6. Similar to previous finding that pH 5.5 showed the highest clipping compared to higher pH formulations. pH 6.8 and pH 7.0 showed low clipping and this finding is similar to the fragmentation of the molecule as measured by HPSEC.

It is hypothesised the high fragmentation rates or clipping seen at low pH (5.5 and 6.0) could be due to protease activity. The molecule can be susceptible to certain enzymes co-eluted during purification which as a result, lead to this chemical degradation of the molecule. To verify this hypothesis, a protease inhibitory study was conducted. The formulation with histidine-arginine HCl at pH 5.5 was selected since it resulted the highest fragmentation and clipping. The HFUS1 material was divided into four portions. The first one was assessed as it is. The second one was spiked with a small level of protease inhibitor (PI) cocktail. The protease inhibitor cocktail was dissolved in DMSO, hence the third portion was spiked in DMSO as control. The last portion was spiked with EDTA. The samples were stored at 40° C. for 4 weeks and the materials were then evaluated by HPSEC for purity and MS for AA clipping. The data is summarised in FIG. 7 and FIG. 8.

The sample with PI or EDTA spiked showed an apparent reduction of fragmentation and AA clipping, while the control and DMSO spiked samples did not show obvious change and both with high level of fragmentation and clipping. This data addressed the hypothesis that proteases are likely the cause of the fragmentation and clipping of the molecule. This enzymatic activity is likely higher at lower pH where the proteases are most effective in cleaving the molecule. Therefore, it is important that the pH of the formulation is maintained at a higher range as demonstrated in the pH optimisation study. In this formulation optimisation study, histidine-arginine HCl system was identified to provide the highest colloidal and conformational stability to HFUS1. The self-association tendency of the molecule was reduced and lower aggregation was seen in this formulation. An optimal pH range was also established to minimise the chemical degradation impact such as fragmentation and AA clipping to the molecule.

### Example 3: Particle Formation Challenges of HFUS1 and Mitigation by Surfactant Screening

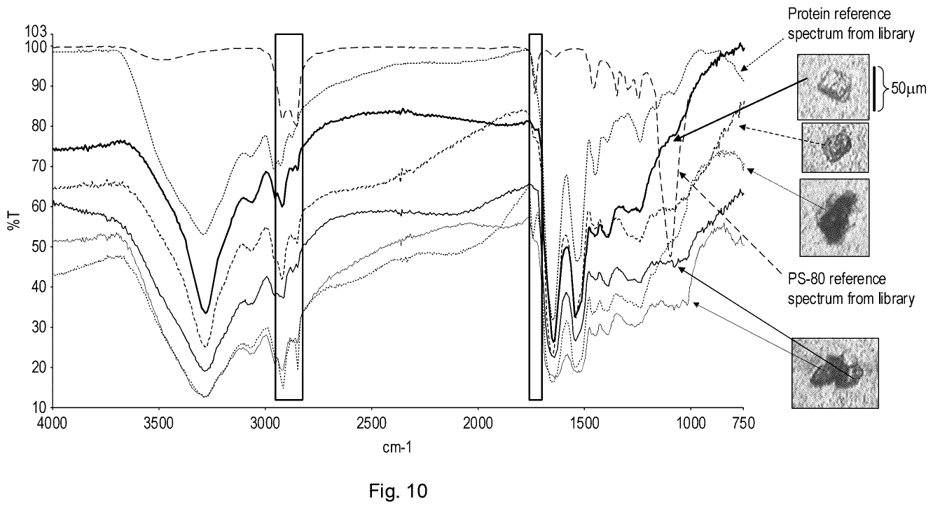

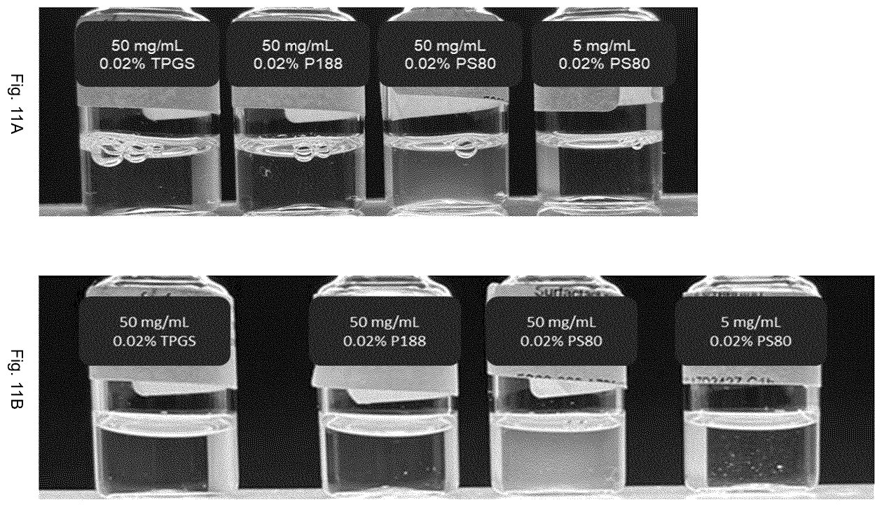

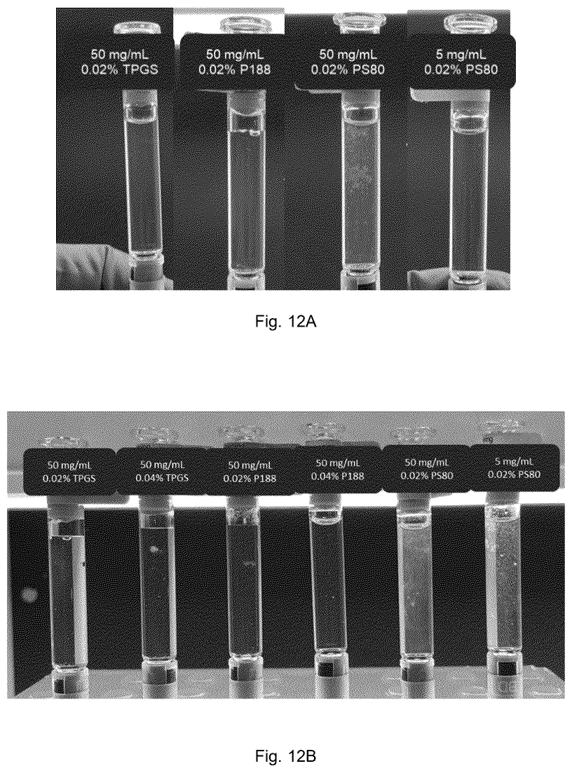

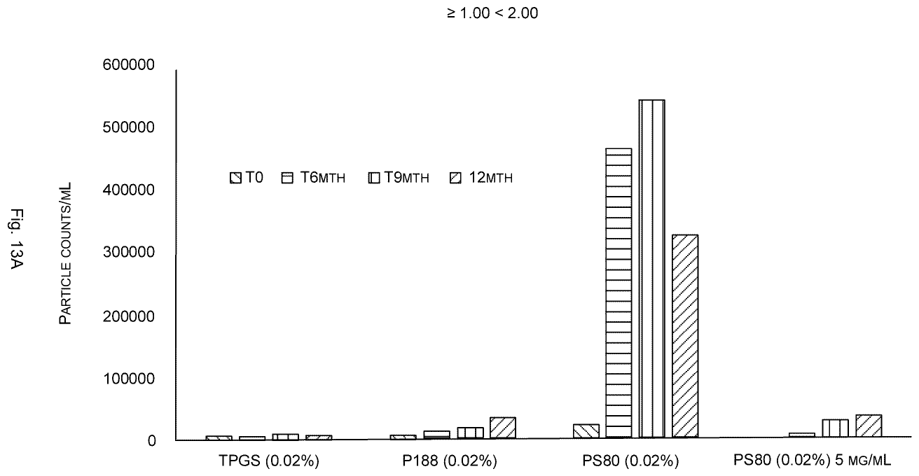

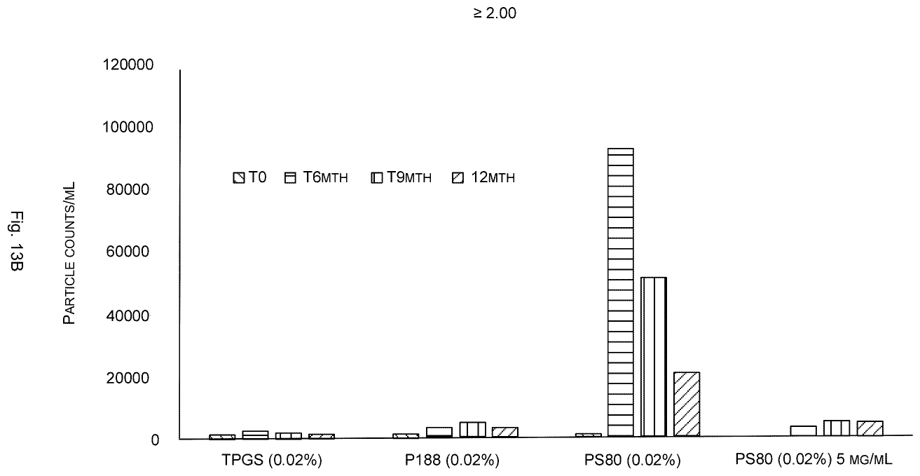

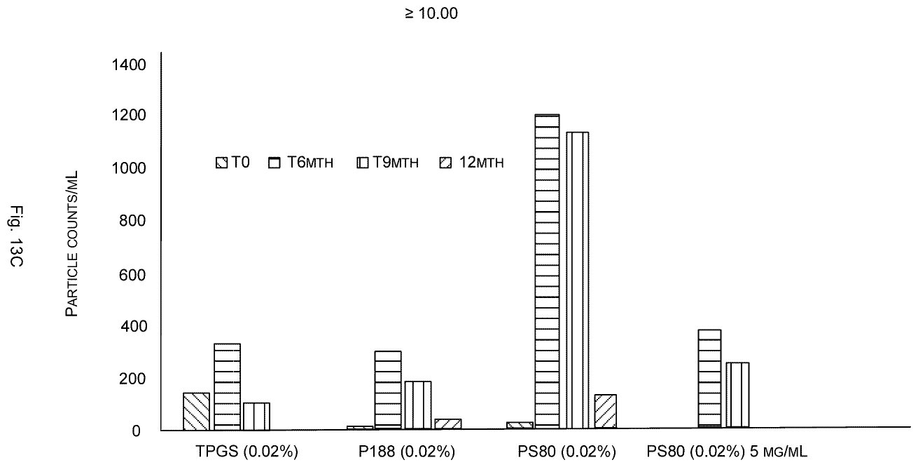

During long term stability storage at 5° C., the formulations from the optimisation studies started to show visible particles. The onset of particles varied from one condition to another. With pH 6.5 and 6.8 formulations started to show particles at 6 months and eventually all other formulations showed particles after longer storage as shown in FIG. 9. Considering that these formulations have various pH, buffer species, and excipients, it was not likely that any of this was the root cause for the particle formation. Therefore, it was suspected that this particle formation could be likely related to the present of PS80, which is a common factor in all the formulations. In the meantime, particle investigation was conducted by using Fourier transform infrared (FTIR) to identify the nature of the particles. FTIR traces are shown in FIG. 10. The particles showed signatures at 3000-2800 cm⁻¹ which is a CH region similar to proteins, and around 1740 cm⁻¹ which is an ester bond region similar to PS80. Though this data is not conclusive, it is clear that the particles are proteinaceous in nature. It is worth noting that in another stability study with a new batch of HFUS1 material (for GLP toxicology study with lead clone), similar particle formation was observed for the histidine-arginine HCl formulation. This indicates that the particles are not just specific to a certain batch of HFUS1 material and they appeared in all HFUS1 materials tested so far.

The host cell protein (HCP) level of the HFUS1 drug substance material was measured. HCPs are process-related protein impurities that are co-purified with the HFUS1 molecule which were not able to be removed by the purification process. The HCP levels of HFUS1 materials are around 100 to 650 ng/mg which are of a high level. Through mass spectrometry-based proteomic identification study, the species of the HCPs were analysed. The HCPs identified included lipoprotein lipase (LPL) which is known to degrade polysorbate, as well as proteases or peptidases which can degrade proteins or clip terminal amino acids. Without wishing to be bound by theory, this led to several hypotheses for the cause of the particle formation which were: 1) LPL degrades polysorbate 80 in the formulation which resulted in impurities such as free fatty acids (FFA). As free fatty acids are insoluble in water, these therefore precipitated out as particles; 2) the free fatty acid impurities could serve as a nucleus to trigger HFUS1 protein aggregation leading to particles and in the meantime, due to degradation, the PS80 level decreased, therefore losing its protection/surfactant effect to prevent particle formation during storage; 3) proteases could degrade HFUS1 and the degraded HFUS1 have exposed patches which are prone for interactions leading to aggregation; and 4) some proteases or HCPs could also interact with HFUS1 causing aggregation to happen.

Considering that the particles identified by FTIR were proteinaceous in nature, it was not likely that they were insoluble FFA only. On the other hand, HFUS1 in the optimised histidine-arginine-HCl formulation (e.g. Formulation 6 of Table 4) did not show abnormal fragmentation or AA clipping, therefore, it was also not likely that the particles were caused by protease degrading HFUS1. In this case, hypotheses 2 and 4 were the most likely mechanism for the HFUS1 particle formation. In order to confirm whether the presence of PS80 play a role in the particle formation, new stability studies were set up replacing PS80 in the histidine-arginine HCl formulation with alternative surfactants, such as poloxamer 188 (P188) and D-α-Tocopherol polyethylene glycol succinate (TPGS). Two different HFUS1 concentrations were tested, i.e. 50 and 5 mg/mL. The formulations were stored in vials and pre-filled syringes (PFS) at 5° C. and examined for particle formation at different timepoints (FIG. 11 and FIG. 12). After 6 months of storage, the 50 mg/ml formulation with PS80 started to show higher opalescence trace of visual particles. The level of particle increased after 9 months and after 12 months, high level of visible particles was observed. Though the 5 mg/mL PS80 formulation did not show particle formation until 9 months' timepoint, visible particles were observed at 12 months. By comparison, formulations containing TPGS and P188 did not show any visible particles after 12 months of storage.

MFI was also used to inspect the sub-visible particles in the HFUS1 formulations. As shown in FIG. 13, the particle counts/mL of the 50 mg/mL formulation with PS80 increased significantly after 6 months, while formulations with TPGS and P188 at 50 mg/mL did not show any significant increase.

The level of PS80 and P188 in the HFUS1 formulations after storage was also tested by HPLC equipped with an evaporative light scattering detector (ELSD). The data is summarised in Table 5. The ELSD data of PS80 formulations was obtained after 5 months while P188 data was after 11 months. The PS80 level dropped below the limit of quantification for the 50 mg/mL HFUS1 formulations, while the 5 mg/mL formulations also showed a decrease of PS80 level. This indicated that PS80 had been degraded in the formulation and, without wishing to be bound by theory, this was likely caused by the LPL present which cleaved the ester bond of PS80 via enzymatic hydrolysis. This also explains why the formulation with lower HFUS1 concentration demonstrated slower PS80 degradation over time. This was likely due to a lower level of co-purifying LPL in the formulation. Conversely, formulations containing P188 in place of PS80 did not show any change in the surfactant level. P188 is a class of water-soluble nonionic triblock copolymers formed by polyethylene oxide (PEO) and polypropylene oxide (PPO) blocks. The PEO and PPO blocks are linked together by an ether bond which is not susceptible to enzymatic hydrolysis by LPL.

TABLE 5
Polysorbate 80 and poloxamer 188 level in the HFUS1 formulations after storage,
as detected by HPLC-ELSD. The limit of quantification (LOQ) is 0.004%.

| | PS80 | | | | | | P188 | |
|---|---|---|---|---|---|---|---|---|
| | 50 mg/mL HFUS1 | | | 5 mg/mL HFUS1 | | | 50 mg/mL HFUS1 | |
| | | 5° C. | 25° C. | | 5° C. | 25° C. | | 5° C. |
| | | (5 | (5 | | (5 | (5 | | (11 |
| | Initial | months) | months) | Initial | months) | months) | Initial | months) |
| Vial | 0.014% | <LOQ | — | 0.020% | 0.015% | 0.011% | 0.023% | 0.023% |
| PFS | 0.014% | <LOQ | <LOQ | 0.020% | 0.015% | 0.011% | 0.023% | 0.023% |

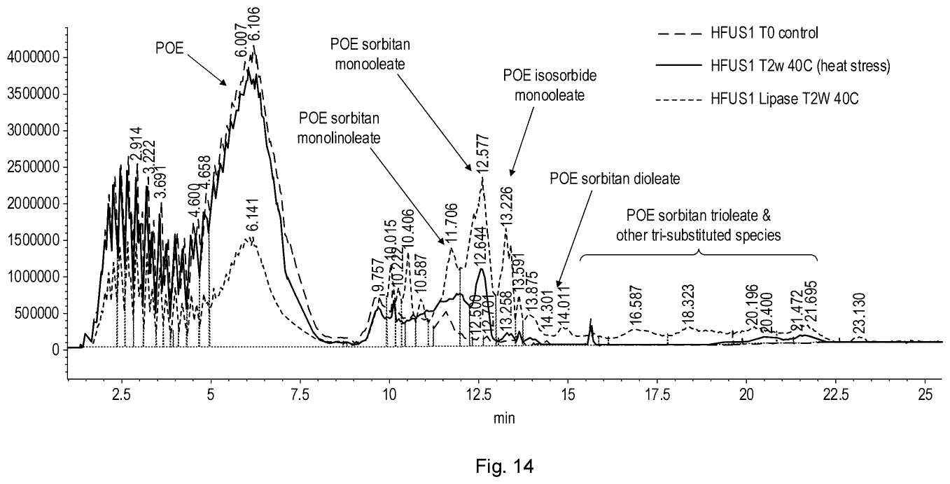

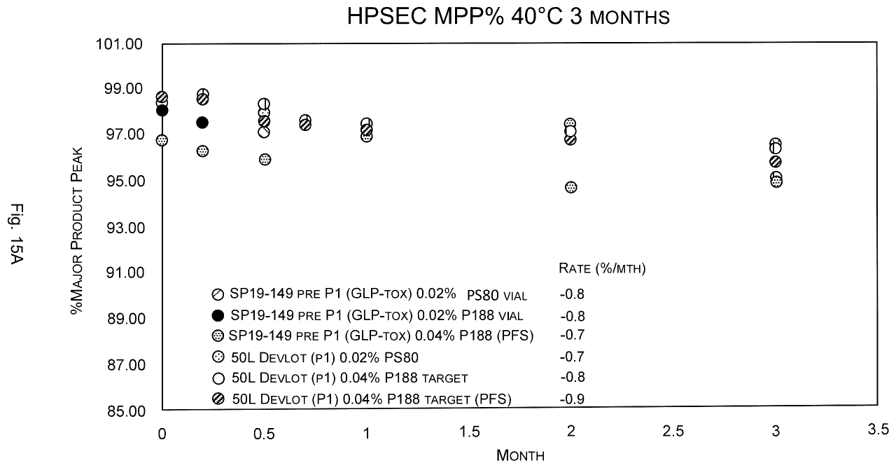

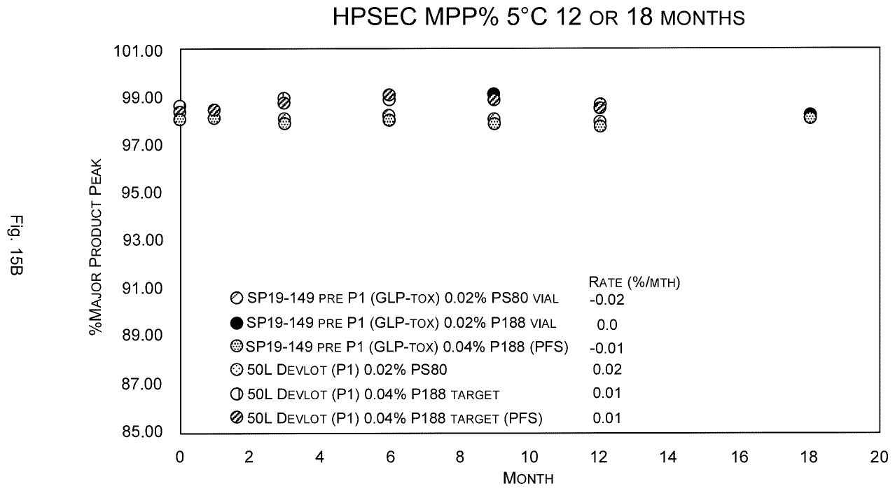

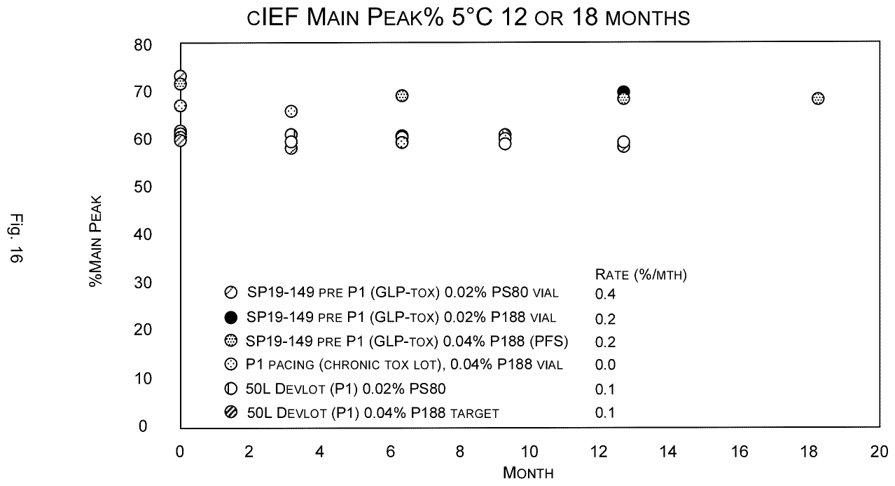

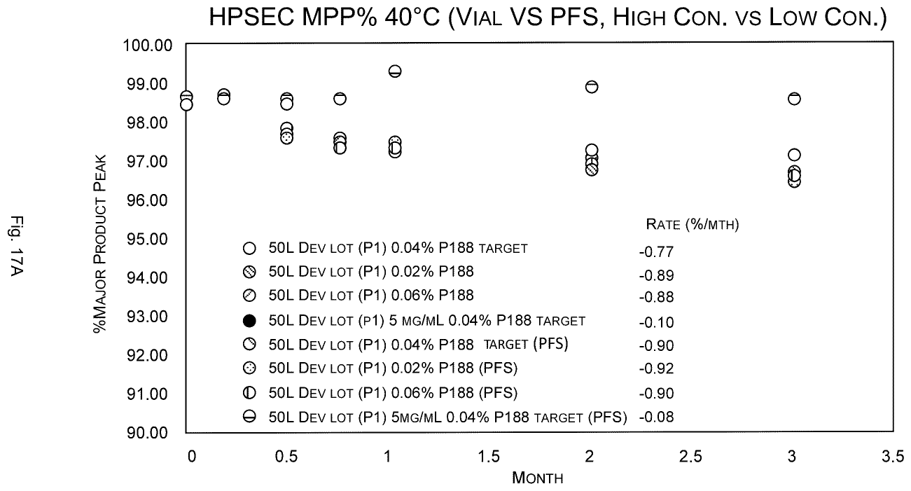

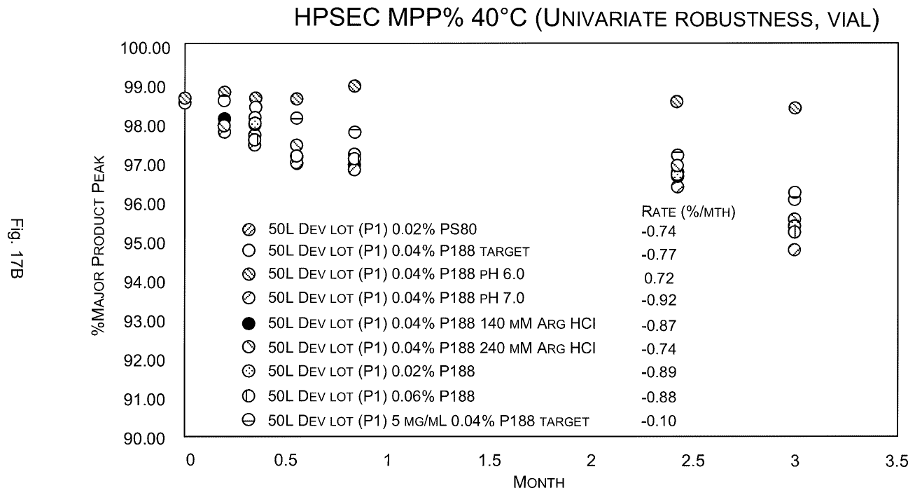

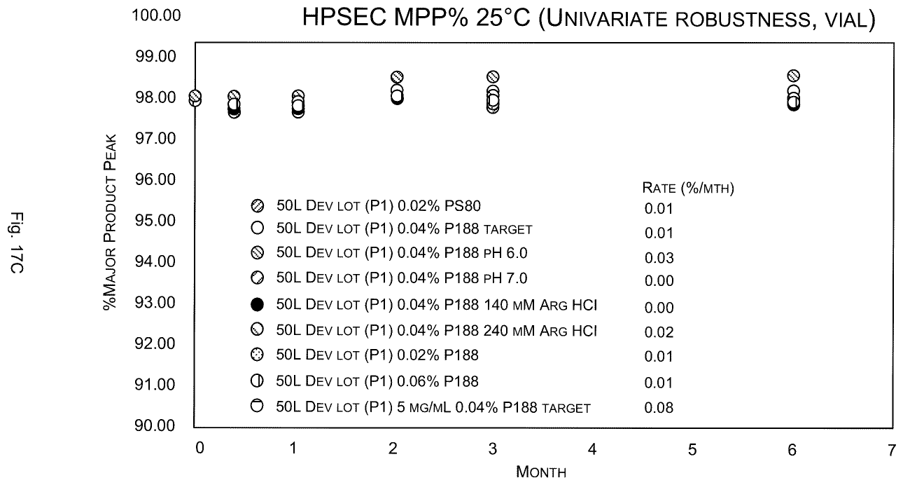

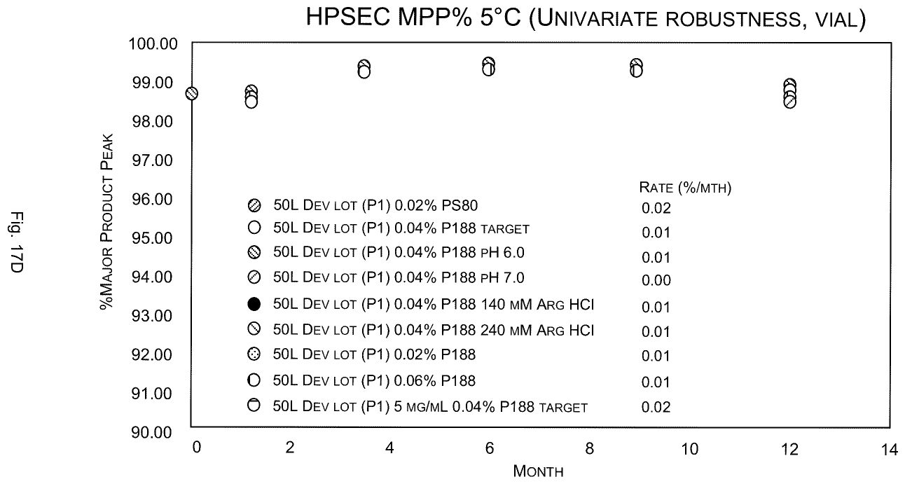

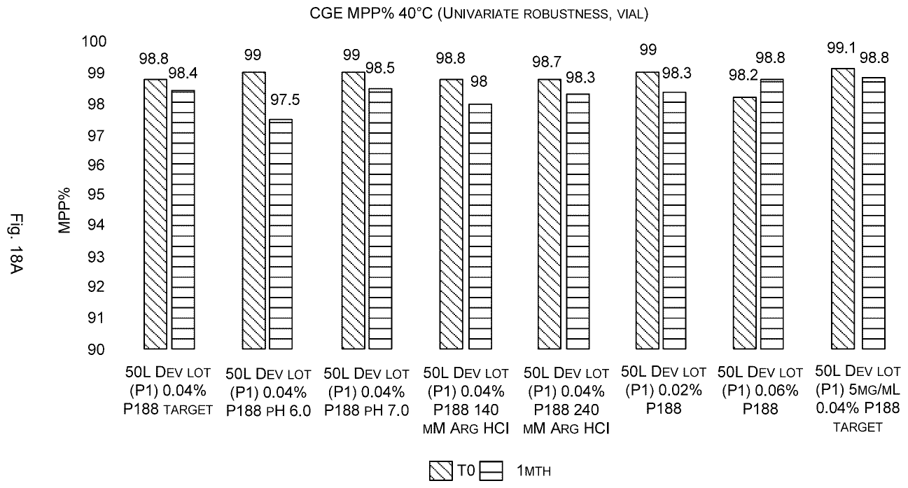

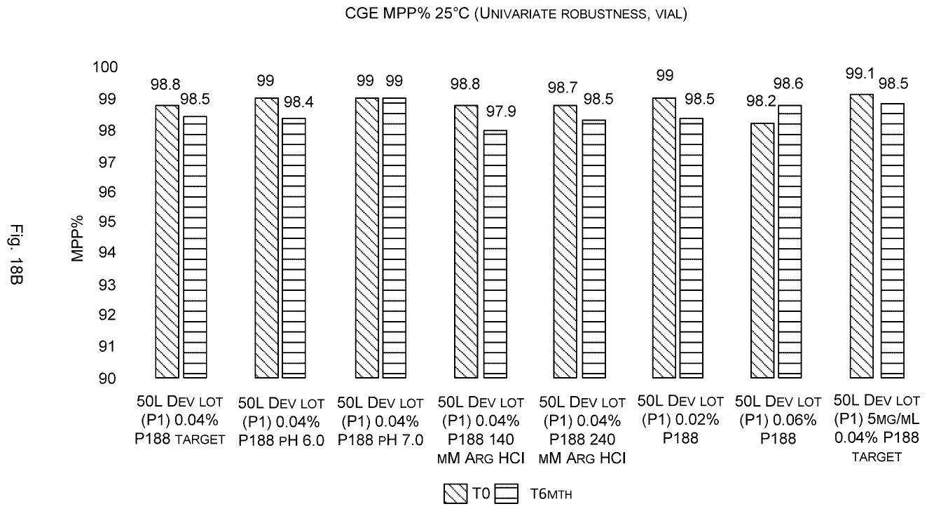

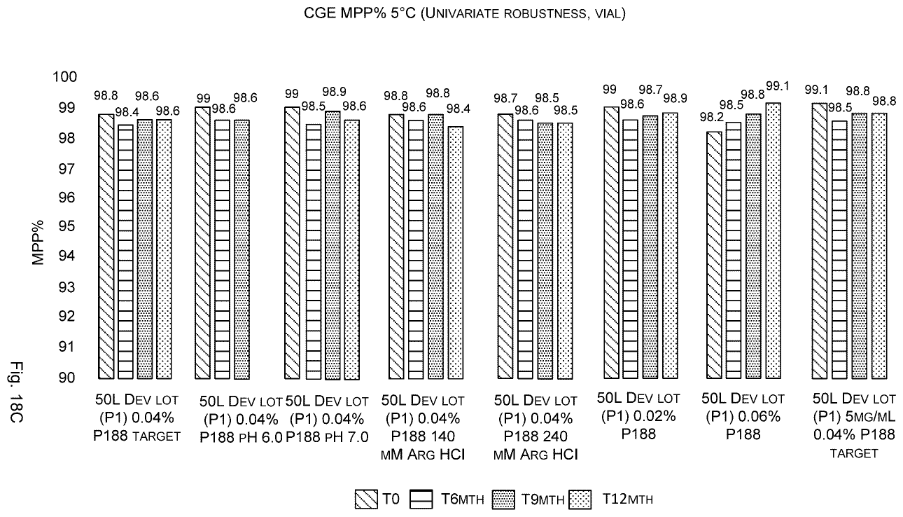

The LPL effect on PS80 degradation was further verified by an LC-MS study. The histidine-arginine HCl formulation containing 50 mg/mL HFUS1 and PS80 (e.g. Formulation 6 of Table 4) was divided into 3 portions. One portion as the initial control sample, another portion was kept at 40° C. for 2 weeks and the last portion was spiked with lipase and also kept at 40° C. for 2 weeks. The LC-MS chromatogram of the samples are shown in FIG. 14. Heat stressed HFUS1 samples exhibited hydrolysis degradation and accumulation of Polyoxyethylene sorbitan monooleate, Polyoxyethylene isosorbide monooleate, and di and tri substituted species. The profile is similar to the lipase spiked sample which showed more significant hydrolysis degradation.

This data and investigation indicate that the polysorbate 80 degradation hypothesis was the cause of particle formation observed with the HFUS1 formulation. Meanwhile the use of alternative, lipase-resistant, surfactants such as P188 or TPGS were able to mitigate the particle formation challenge.

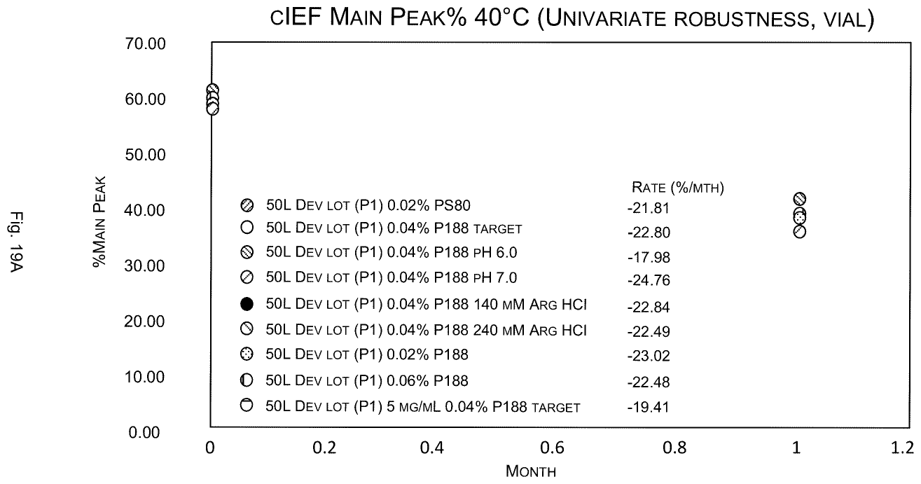

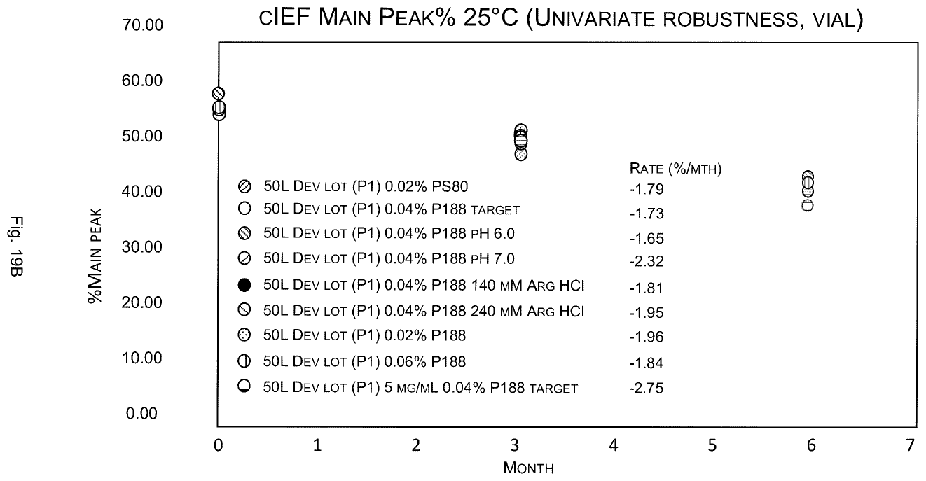

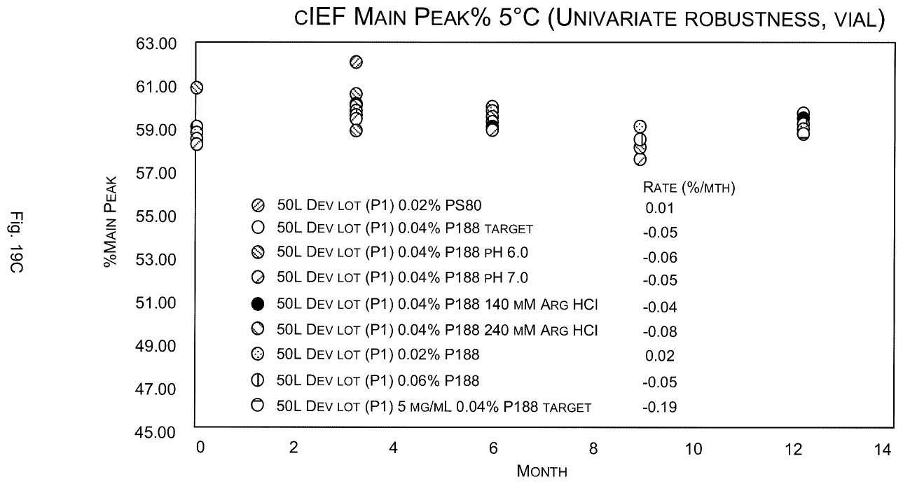

Apart from the visible particle inspection and sub-visible particle analysis, HPSEC and cIEF were also used to evaluate the purity as well as chemical degradation profiles of the HFUS1 formulations with different surfactants. The HPSEC data is shown in FIG. 15 and cIEF data in FIG. 16. Formulations containing P188 and PS80 showed similar degradation profile and additionally, no obvious difference was seen in samples stored in vials or pre-filled syringes (PFS). HFUS1 is thus stable in the histidine-arginine HCl formulation with P188 with no obvious change in both the physical and chemical stability after storage for 18 months.

### Example 4: Robustness of the HFUS1 Formulation

After formulation optimisation, which was focused on the buffer species, pH and excipient, and surfactant screening, an optimized formulation in histidine buffer containing arginine-HCl and poloxamer 188 at pH 6.5 was developed. This formulation provided long term protection to HFUS1 with low chemical and physical degradation. In this study, the robustness of this formulation system was evaluated. This was studied by varying the concentration of arginine-HCl and surfactant, as well as the pH. The different formulations were stored at 5, 25 and 40° C. to evaluate their stability profiles. A summary of the different conditions tested is given in Table 6. The formulations were contained in either 2R glass vials or PFS.

TABLE 6
Summary of formulations in the robustness study

| Container | Number | Formulation |
|---|---|---|
| 2R vial | F0 | 50 mg/mL HFUS1, 20 mM Histidine/Histidine HCL, 190 mM |
| | (control) | arginine HCl, pH 6.5, 0.02% (w/v) PS80 |
| | F1 | 50 mg/mL HFUS1, 20 mM Histidine/Histidine HCL, 190 mM |
| | (Target) | Arginine HCl, pH 6.5, 0.04% (w/v) P188 |
| | F2 | 50 mg/mL HFUS1, 20 mM Histidine/Histidine HCL, 190 mM |
| | | Arginine HCl, pH 6.0, 0.04% (w/v) P188 |
| | F3 | 50 mg/mL HFUS1, 20 mM Histidine/Histidine HCL, 190 mM |
| | | Arginine HCl, pH 7.0, 0.04% (w/v) P188 |
| | F4 | 50 mg/mL HFUS1, 20 mM Histidine/Histidine HCL, 140 mM |
| | | Arginine HCl, pH 6.5, 0.04% (w/v) P188 |
| | F5 | 50 mg/mL HFUS1, 20 mM Histidine/Histidine HCL, 240 mM |
| | | Arginine HCl, pH 6.5, 0.04% (w/v) P188 |
| | F6 | 50 mg/mL HFUS1, 20 mM Histidine/Histidine HCL, 190 mM |
| | | Arginine HCl, pH 6.5, 0.02% (w/v) P188 |
| | F7 | 50 mg/mL HFUS1, 20 mM Histidine/Histidine HCL, 190 mM |
| | | Arginine HCl, pH 6.5, 0.06% (w/v) P188 |
| | F8 | 5 mg/mL HFUS1, 20 mM Histidine/Histidine HCL, 190 mM |
| | | Arginine HCl, pH 6.5, 0.04% (w/v) P188 |
| 1 mL PFS | F1P | 50 mg/mL HFUS1, 20 mM Histidine/Histidine HCL, 190 mM |
| | | Arginine HCl, pH 6.5, 0.04% (w/v) P188 |
| | F6P | 50 mg/mL HFUS1, 20 mM Histidine/Histidine HCL, 190 mM |
| | | Arginine HCl, pH 6.5, 0.02% (w/v) P188 |
| | F7P | 50 mg/mL HFUS1, 20 mM Histidine/Histidine HCL, 190 mM |
| | | Arginine HCl, pH 6.5, 0.06% (w/v) P188 |
| | F8P | 5 mg/mL HFUS1, 20 mM Histidine/Histidine HCL, 190 mM |
| | | Arginine HCl, pH 6.5, 0.04% (w/v) P188 |

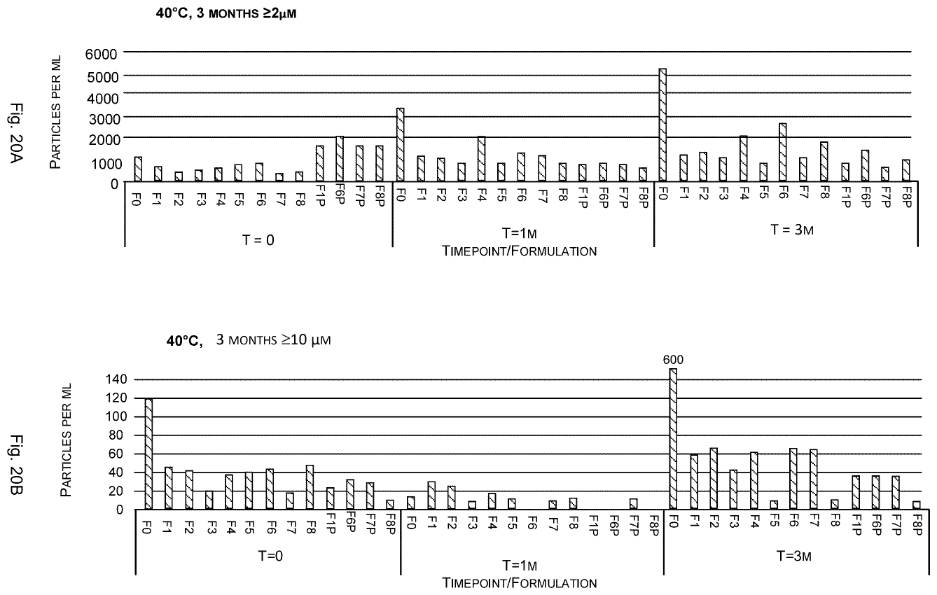

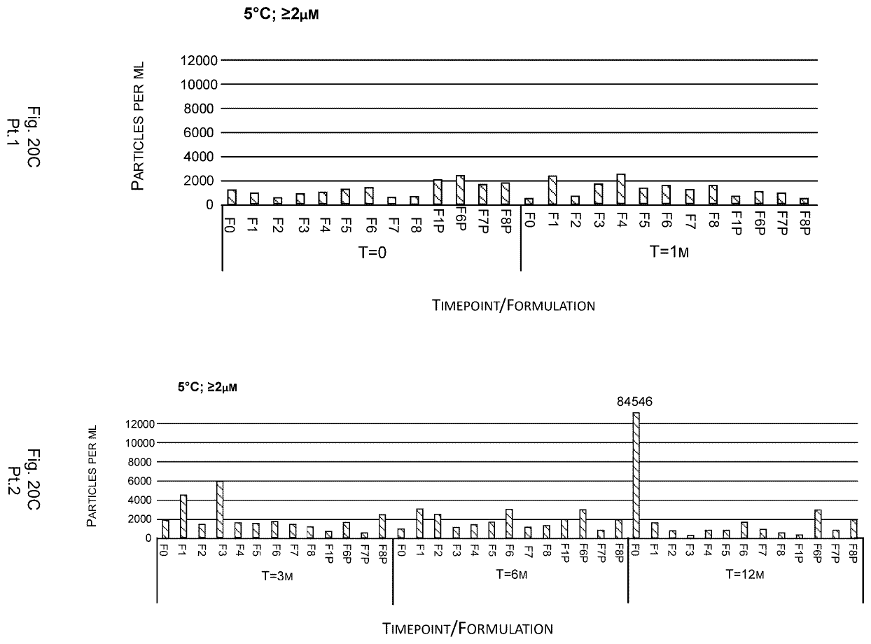

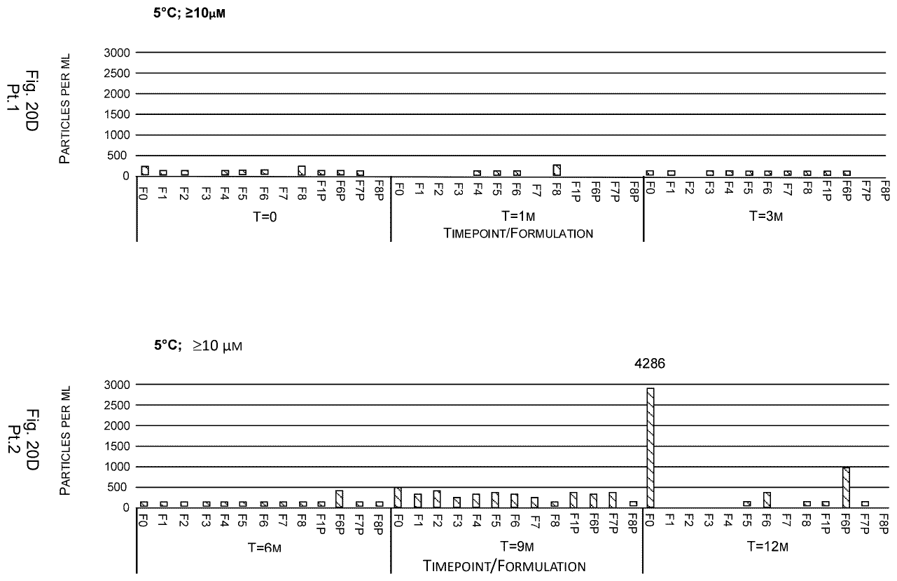

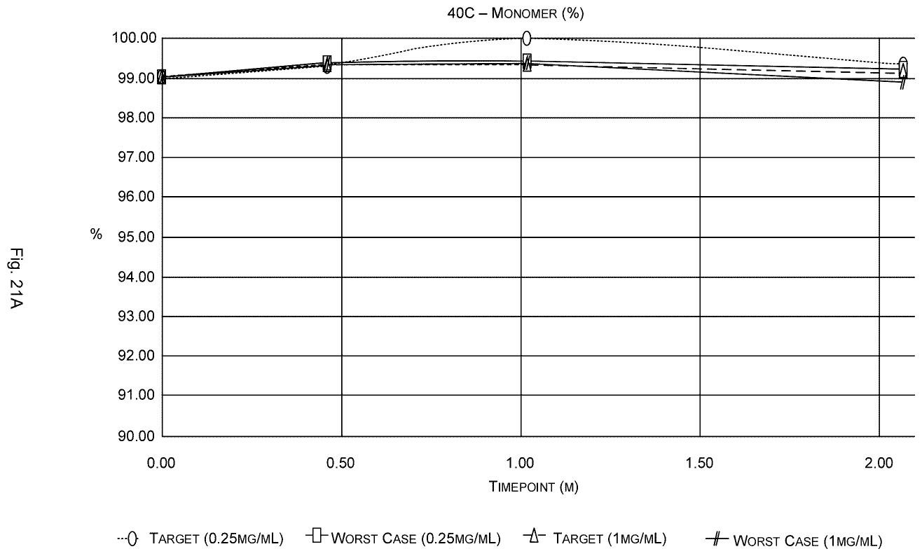

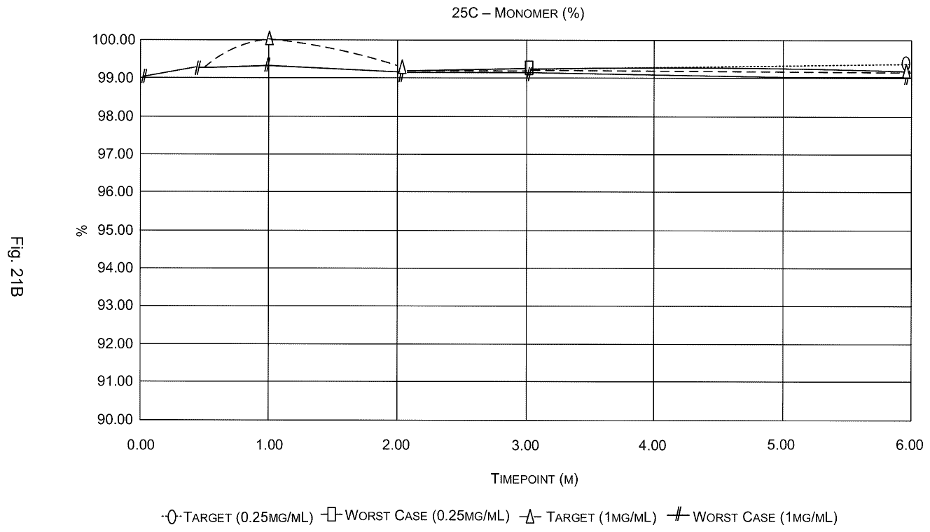

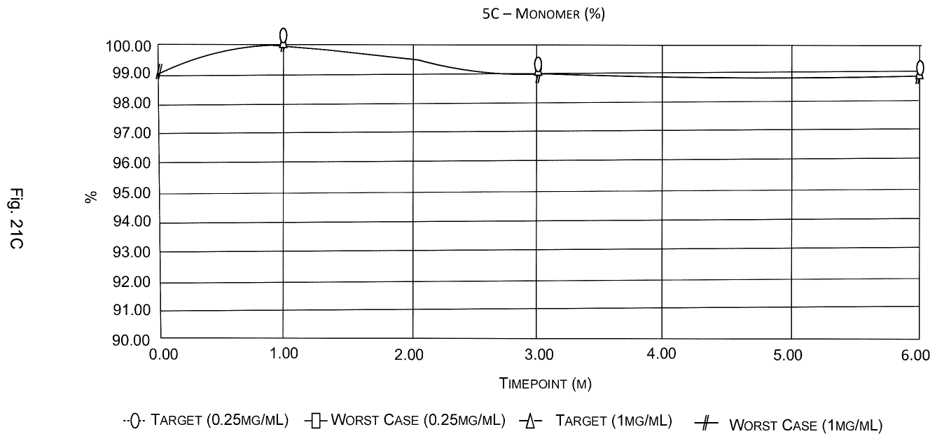

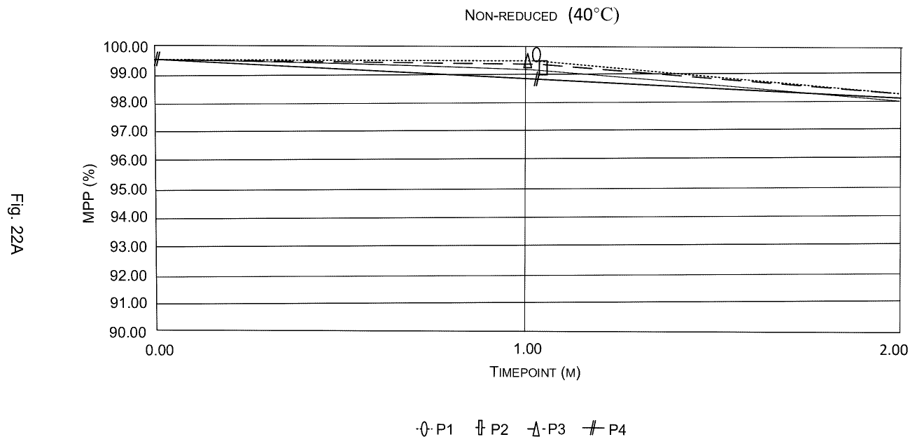

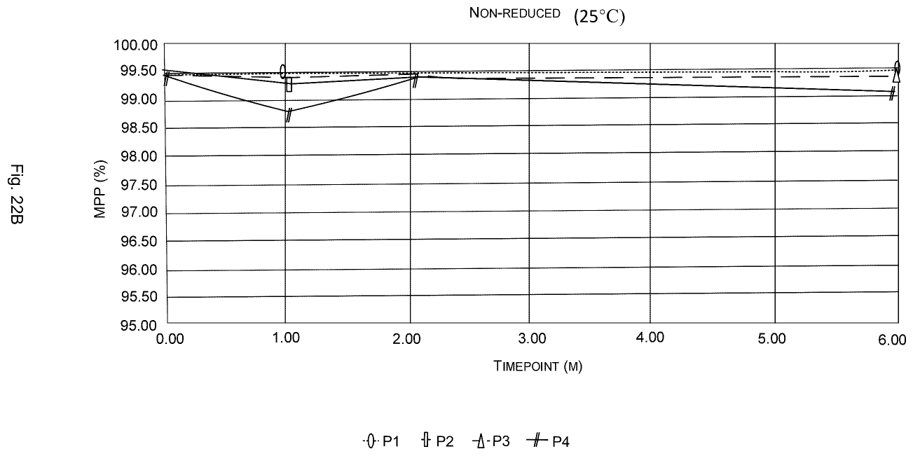

The formulations were tested by HPSEC, CGE, cIEF and MFI and data summarised in FIG. 17, FIG. 18, FIG. 19 and FIG. 20. HPSEC was used to evaluate the purity and aggregation of HFUS1 during storage. Meanwhile, CGE was used to detect any fragmentation of the molecule and cIEF for chemical instability. The subvisible particles were assessed by MFI, and visual inspection of the formulations was also performed (data not shown).

No particle formation issue was seen by MFI and visual inspection after storage at 40° C. for 3 months and 5° C. for over 12 months. And as a comparison, the control formulation containing PS80 instead of P188 showed high levels of sub-visible particles and visual particles. This data further confirms that the use of P188 mitigated the particle issues observed with the PS80 system. The formulations did not show obvious change of the Major Product Peak (MPP %) detected by HPSEC after storage at 5° C. for over 12 months and 25° C. for over 6 months, indicating good physical stability of the formulations. The rate of MPP % decrease at 40° C. showed small differences between the different formulation conditions, however, the rate was kept very low (<1% decrease per month). The concentration of HFUS1 has an impact on the aggregation of the molecule with 5 mg/ml sample showing a much lower MPP % decrease per month (FIG. 17). The chemical stability of the formulations were evaluated by CGE and cIEF. For CGE, minimal or neglectable MPP % decrease was seen for the formulations after storage. This indicated a low level of fragmentation of the molecule. Meanwhile, cIEF data also showed neglectable change of MPP % after storage at 5° C. for 12 months indicated low chemical instability with the molecule. The MPP % decrease at 25 and 40° C. was more obvious but all remained an acceptable level. In general, the histidine-arginine HCl based formulation showed excellent stability and robustness. Formulation F1/F1P was selected as the overall favoured formulation.

### Example 5: Formulation Stability at Lower HFUS1 Concentrations

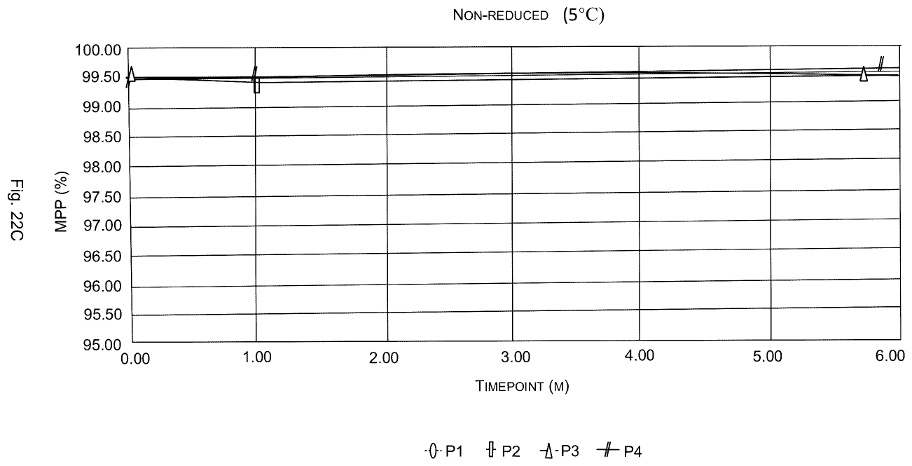

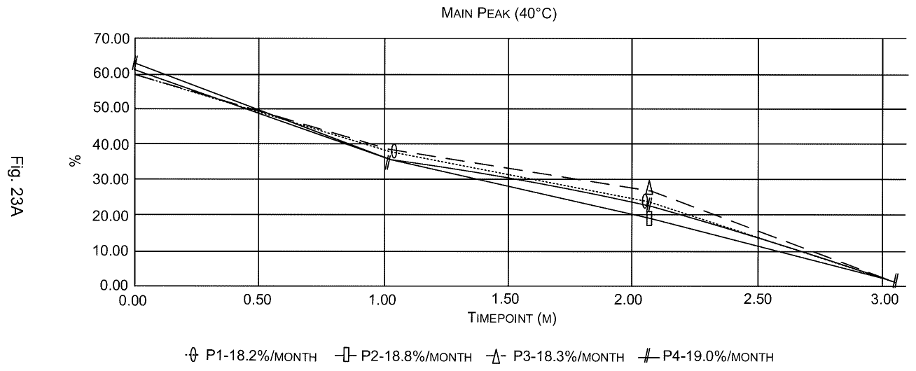

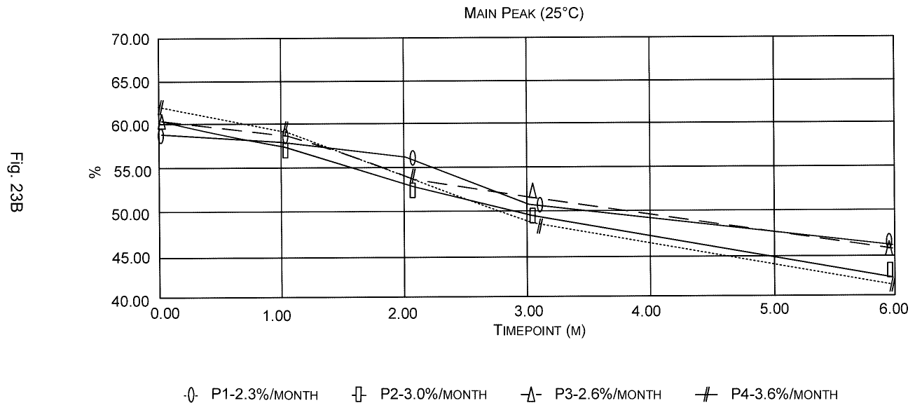

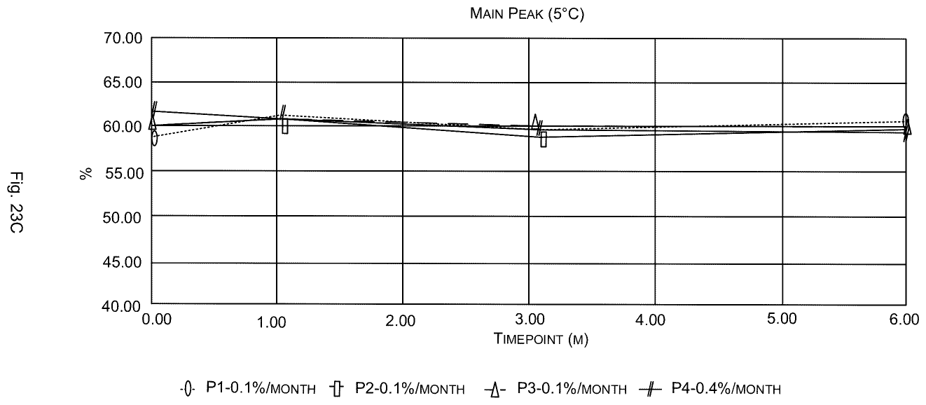

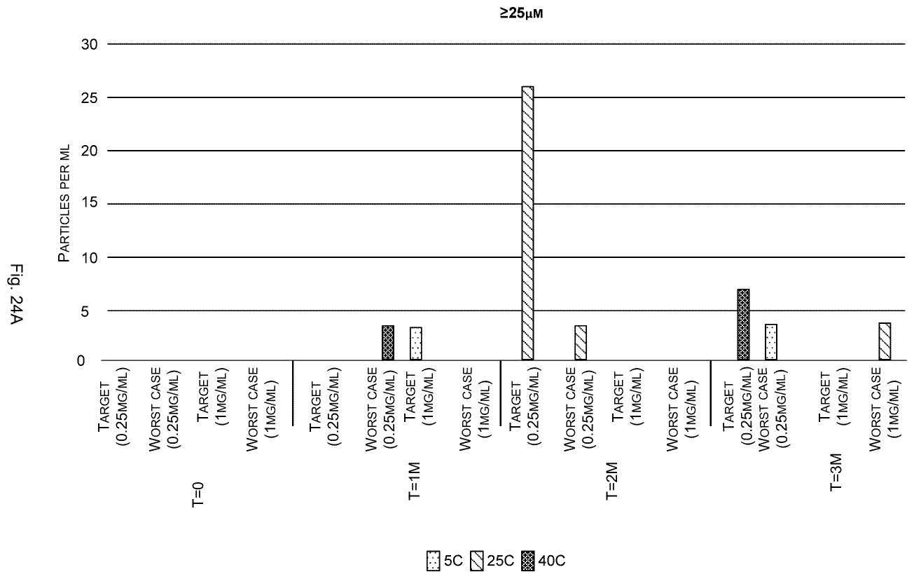

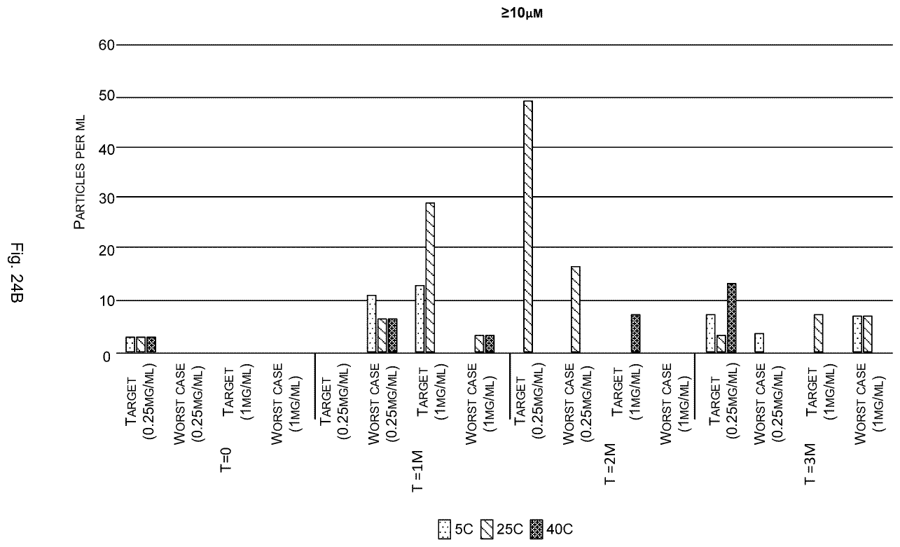

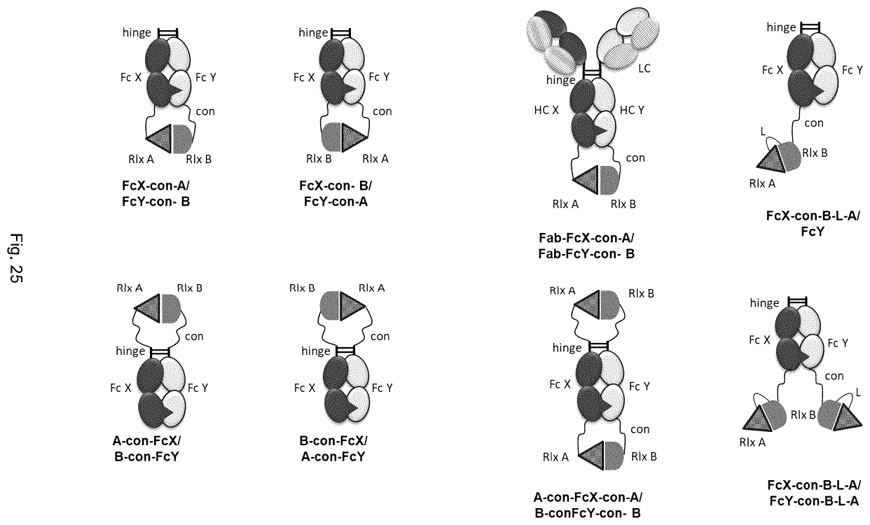

The stability of the HFUS1 formulation at lower protein concentrations were also studied. The formulations were filled in the 1 mL PFS and stored at 5, 25 and 40° C. for testing at multiple timepoints. A summary of the formulation condition is shown in Table 7. The concentrations evaluated were 0.25 mg/mL and 1 mg/mL HFUS1 and they were formulated in the target formulation (P1 and P3) and also in a formulation that would likely generate a worse case stability (P2 and P4), which contained lower Arginine HCl concentration and a higher pH. In the robustness study described in Example 4, higher aggregation was seen with a lower Arginine HCl level and pH 7.0 sample showed higher degradation rate by cIEF. This study in Example 5 was to allow generation of formulation data at lower HFUS1 concentrations to support the suitability of the formulation across a wide range of HFUS1 concentrations. The formulations were tested by visual inspection (data not shown), MFI, HPSEC, cIEF and CGE and the data are summarised in FIG. 21 to FIG. 24.

TABLE 7
HFUS1 formulation conditions

| Container | Number | Formulation |
|---|---|---|
| 1 mL PFS | P1 | 0.25 mg/mL HFUS1, 20 mM Histidine/Histidine HCL, 190 mM |
| | | Arginine HCl, pH 6.5, 0.04% (w/v) P188 |
| | P2 | 0.25 mg/mL HFUS1, 20 mM Histidine/Histidine HCL, 140 mM |
| | | Arginine HCl, pH 7.0, 0.04% (w/v) P188 |
| | P3 | 1 mg/mL HFUS1, 20 mM Histidine/Histidine HCL, 190 mM Arginine |
| | | HCl, pH 6.5, 0.04% (w/v) P188 |
| | P4 | 1 mg/mL HFUS1, 20 mM Histidine/Histidine HCL, 140 mM Arginine |
| | | HCl, pH 7.0, 0.04% (w/v) P188 |

HPSEC data indicated that after storage for 6 months at 5 and 25° C., and 3 months at 40° C., HFUS1 remained stable with no monomer % loss at all temperatures tested. No apparent aggregation was observed. CGE was used to monitor any fragmentation of the molecule during storage and no purity loss was observed at 5 and 25° C., and only minimal loss at 40° C. cIEF was used to detect any chemical degradation as indicated by the change of the main peak. Around 18% monthly loss of main peak was observed after 40° C. storage. This was lower than the rate observed at the higher concentration formulations (e.g. 50 mg/mL and 5 mg/mL). While the degradation at 25° C. was slightly higher than the 50 mg/ml and 5 mg/mL formulations. These degradation rates at 25 and 40° C. are at an acceptable level and importantly, the degradation rate was minimal at 5° C. Almost no change was observed after 6 months of storage. Particle formation tendency was also evaluated by visual inspection (visible particle) and MFI (sub-visible particle). The particle level of HFUS1 in the formulation remained low during storage. Normally, at a lower protein concentration formulation, aggregation and particle risk is lower while chemical degradation risk is higher. This data set at low concentrations indicate that HFUS1 has no chemical or physical stability issues at the concentrations studied.

### Example 6: Formulation Stability at HFUS1 Concentration of 33 mg/mL

After the development of the optimized formulation, univariate robustness stability studies were conducted at 33 mg/ml in the 1 mL PFS. PFS was selected as the intended long term storage container for this molecule.

The concentration of 33 mg/mL corresponds to the highest concentration within the ±10% range of a 30 mg/mL formulation. Stability risk related to subvisible particle generation is considered the highest at the highest protein concentration.

In this study, the formulation's robustness at the lower and upper end of specifications for the excipient, the surfactant, and the pH was evaluated. This was studied by varying the concentration of arginine-HCl and surfactant, as well as the pH. The different formulations were stored at 5, 25 and 40° C. and tested at multiple timepoints to evaluate their stability profiles. A summary of the different conditions tested is given in Table 8.

TABLE 8
Formulation compositions of the robustness
stability study arms conducted in 1 mL PFS

| Number | Formulation |
|---|---|
| PFS - Control | 33 mg/mL HFUS1, 20 mM Histidine/Histidine HCL, 190 mM Arginine HCl, |
| | pH 6.5, 0.04% (w/v) P188 (target/control) |
| PFS - 1 | 33 mg/mL HFUS1, 20 mM Histidine/Histidine HCL, 190 mM Arginine HCl, |
| | pH 6.0, 0.04% (w/v) P188 |
| PFS - 2 | 33 mg/mL HFUS1, 20 mM Histidine/Histidine HCL, 190 mM Arginine HCl, |
| | pH 7.0, 0.04% (w/v) P188 |
| PFS - 3 | 33 mg/mL HFUS1, 20 mM Histidine/Histidine HCL, 150 mM Arginine HCl, |
| | pH 6.5, 0.04% (w/v) P188 |
| PFS - 4 | 33 mg/mL HFUS1, 20 mM Histidine/Histidine HCL, 230 mM Arginine HCl, |
| | pH 6.5, 0.04% (w/v) P188 |
| PFS - 5 | 33 mg/mL HFUS1, 20 mM Histidine/Histidine HCL, 190 mM Arginine HCl, |
| | pH 6.5, 0.02% (w/v) P188 |
| PFS - 6 | 33 mg/mL HFUS1, 20 mM Histidine/Histidine HCL, 190 mM Arginine HCl, |
| | pH 6.5, 0.06% (w/v) P188 |

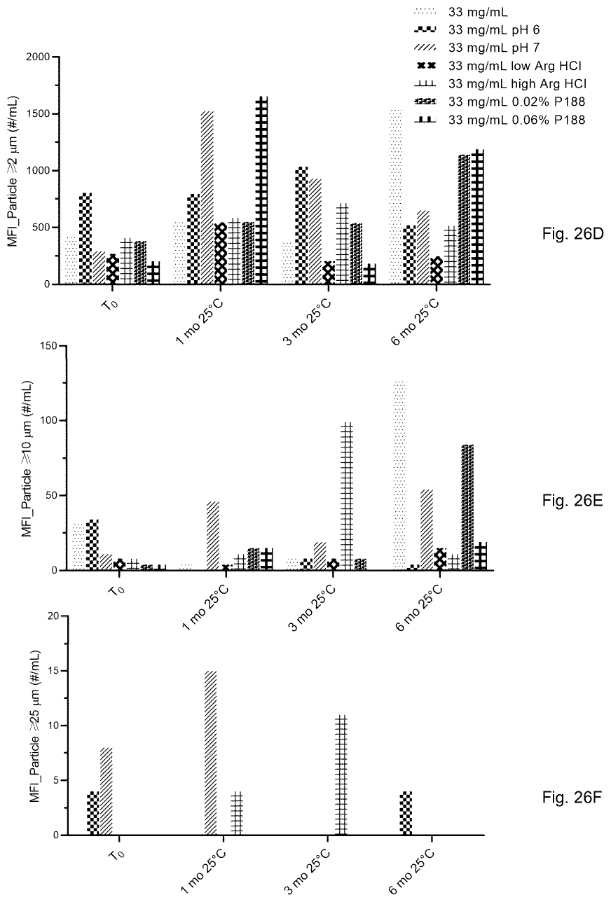

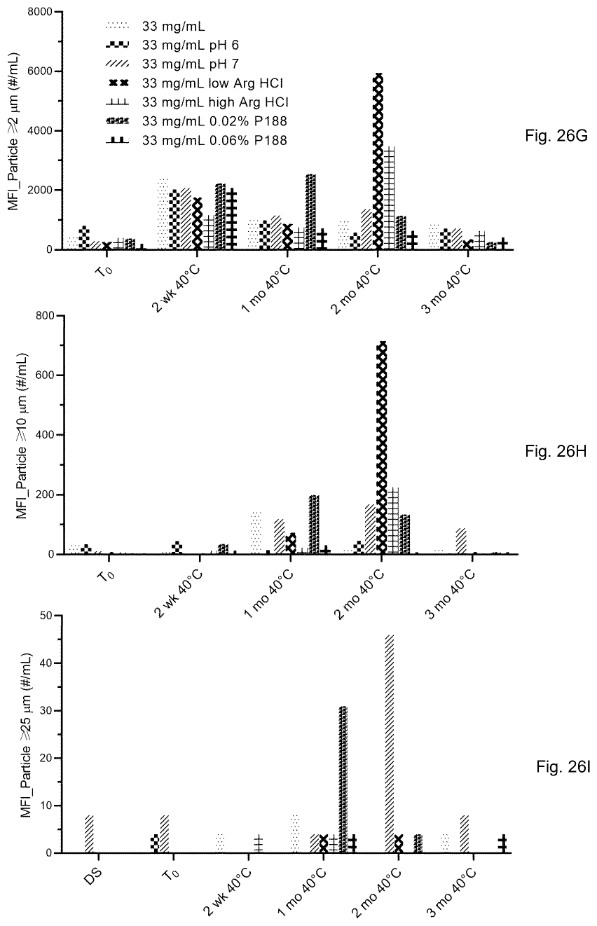

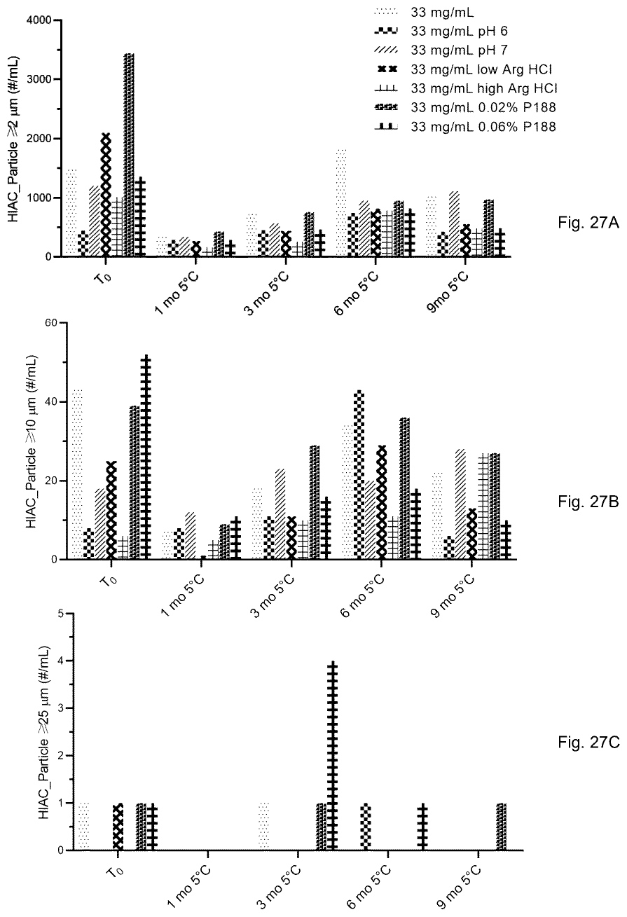

The formulations were tested by MFI, HIAC, HPSEC, cIEF, potency, and RP-HPLC and the data is summarised in FIG. 26 to FIG. 31.

The subvisible particles were assessed by MFI, HIAC and visual inspection of the formulations was also performed (data not shown). HIAC is the compendial method for subvisible particles counts and MFI is used as an orthogonal method. HPSEC was used to evaluate the purity of HFUS1 during storage. Meanwhile, cIEF was used to detect any chemical degradation as indicated by the change of the main peak. A potency assay was used to assess the binding of the HFUS1 to the RXFP1 receptor over time points. RP-HPSEC was used to monitor amino-acid (AA) clipping at the C-terminus of chain B of relaxin.

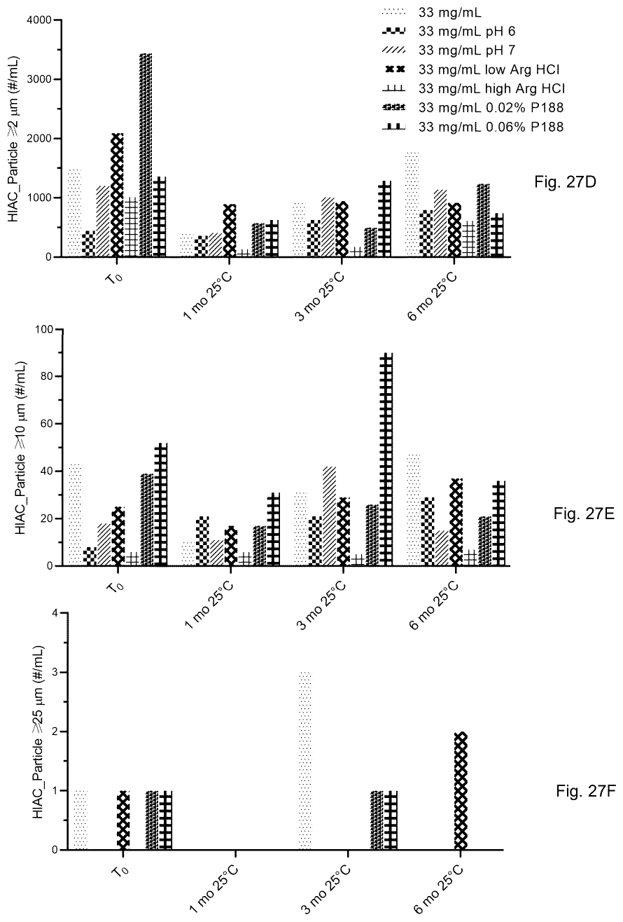

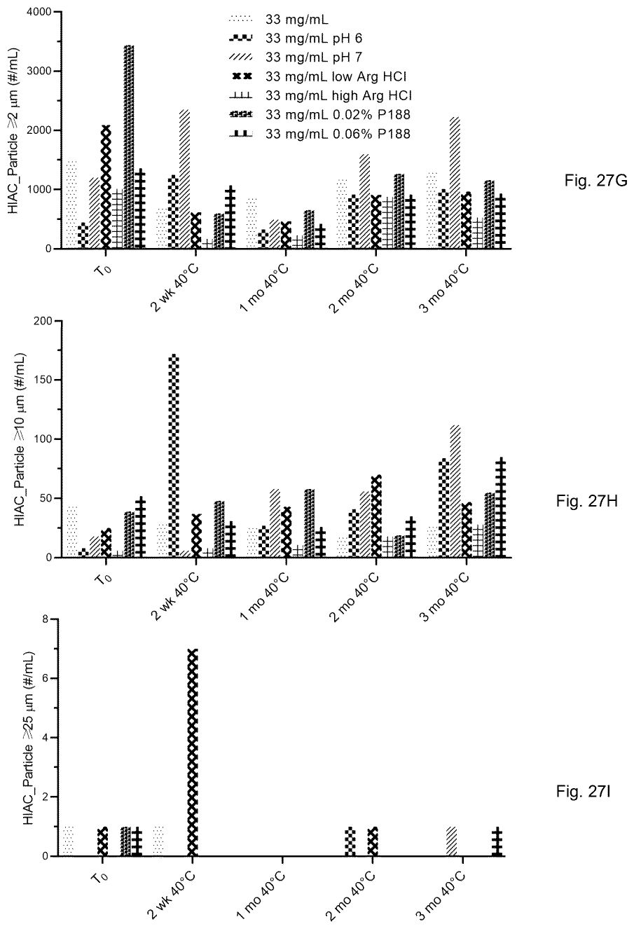

No trends in particle formation were observed by MFI or visual inspection after storage at 40° C. for 3 months, 25° C. for 6 months, or 5° C. for 9 months (FIG. 26). HIAC data after storage for the same duration showed no trend in subvisible particles counts across all conditions, and subvisible particles counts are well within the USP limits (particle size of ≥10 μm and ≥25 μm not exceeding 6000 and 600 respectively; Pharmacopeia US. 2014. USP 787 and 788) for all the conditions tested (FIG. 27). This data further confirms that the use of P188 mitigated the particle issues observed with the PS80 system.

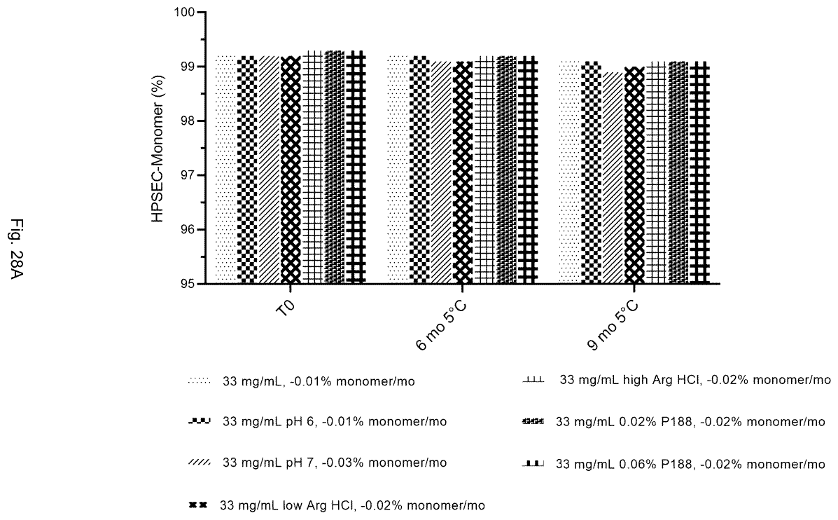

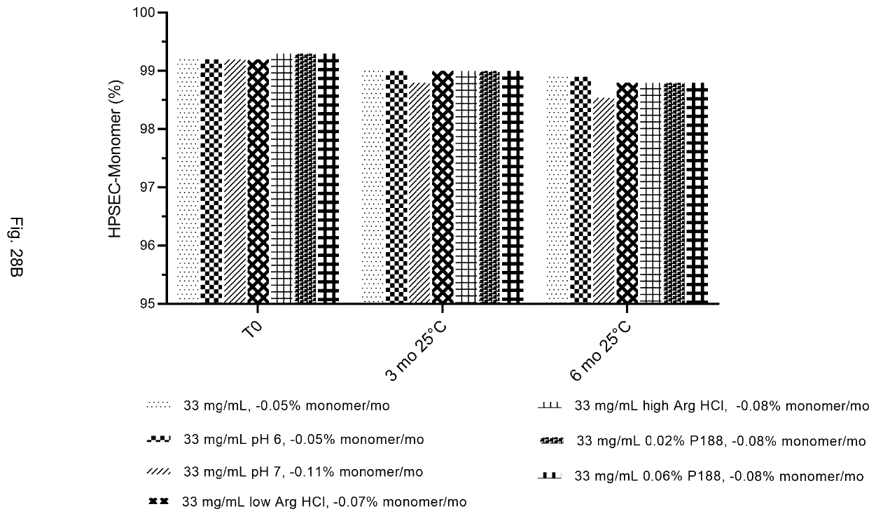

HPSEC data indicated that after storage for 9 months at 5° C., all formulations of HFUS1 remained stable with no monomer loss. Higher monomer loss was observed at pH 7 as compared to pH 6 after storage for 6 months at 25° C. (−0.11%/mo vs −0.05%/mo) and 3 months at 40° C. (−0.71%/mo vs −0.36%/mo). These data indicate good physical stability of the formulations, with no monomer loss at the intended long term storage temperature of −5° C. and <1% decrease per month for the other two temperatures (FIG. 28).

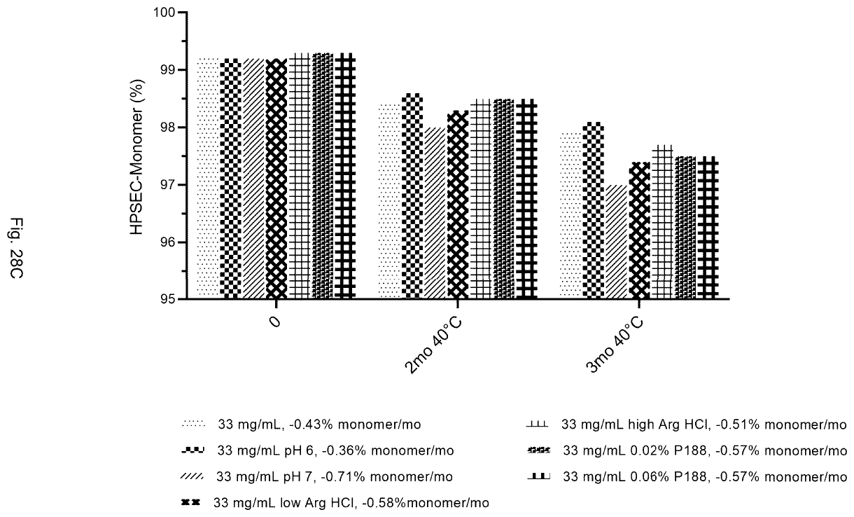

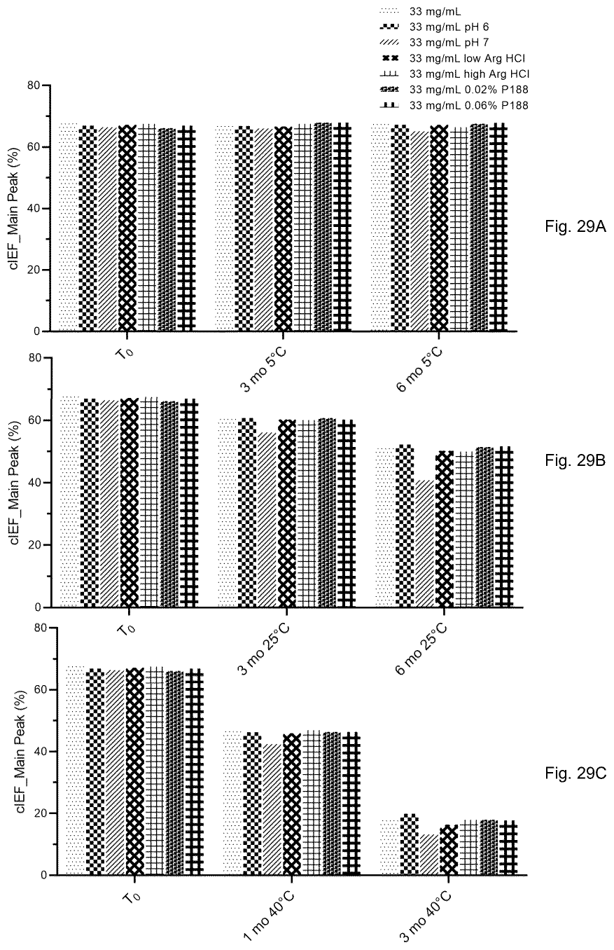

cIEF data also showed negligible change of the main peak after storage at 5° C. for 6 months, indicating low chemical instability of the molecule. The main peak decrease at 25° C. and 40° C. was more obvious, with pH 7 showing the highest rate of change as compared to the other formulation conditions (FIG. 29). Changes in the cIEF profile are expected under these stressed and accelerated conditions that the molecule would not be exposed to during its shelf life, and the degradation rate was minimal at 5° C. for all formulations tested.

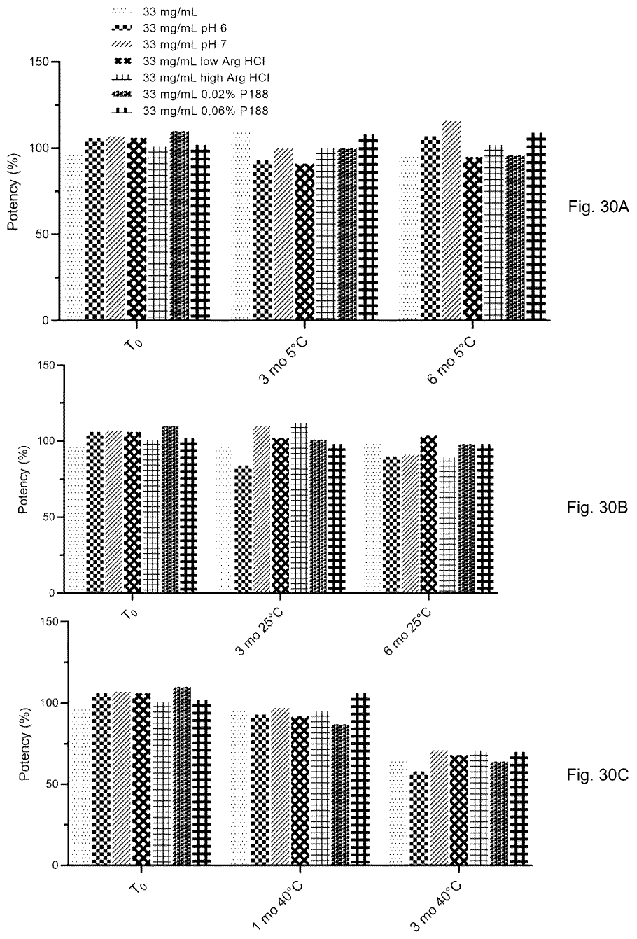

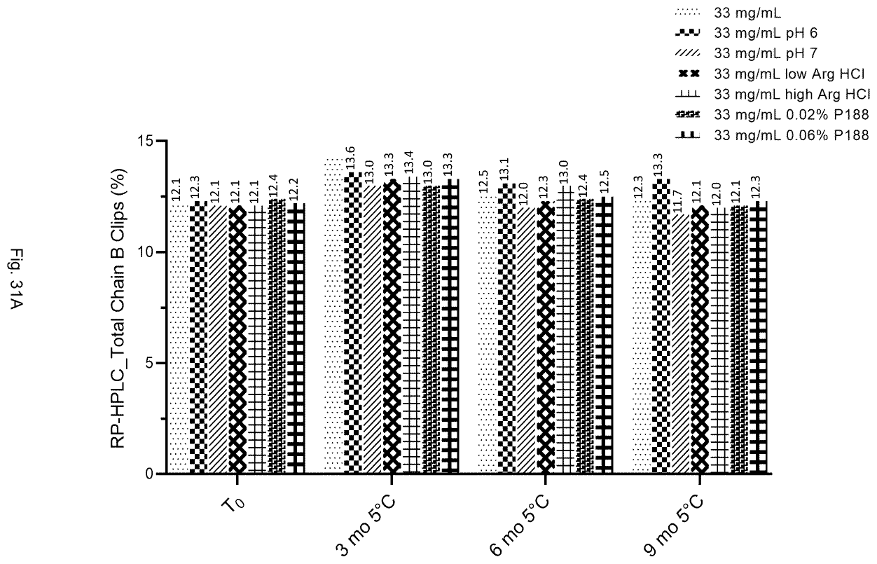

Potency results indicated no change in molecule activity after storage at 5° C. for 6 months for all formulation compositions tested. The change in potency at the other two temperatures, 25° C. and 40° C., was similar for all formulations (FIG. 30).

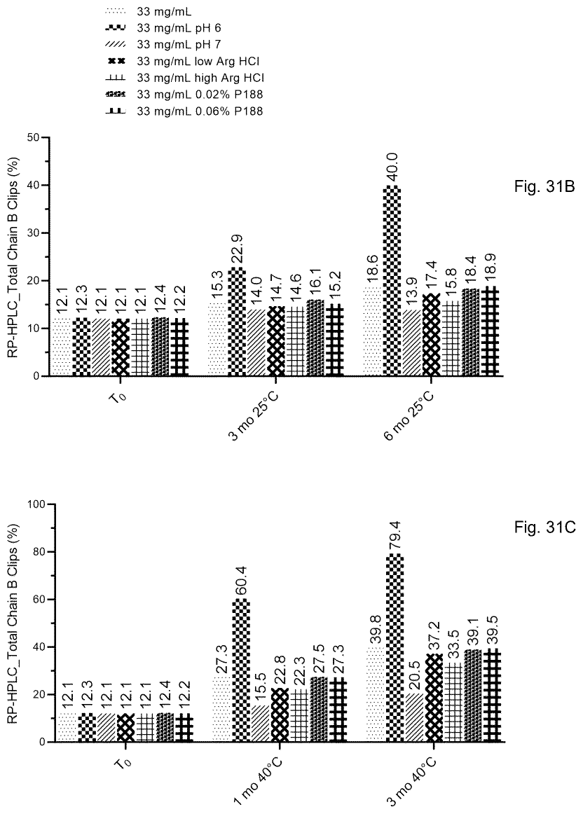

RP-HPLC results at 5° C. showed no change in the major product peak for all the formulations up to 9 months. The pH 6 formulation showed higher chain B clipping as compared to the higher pH 7 formulation at 25° C. and 40° C. (FIG. 31).

### CONCLUSION

During formulation development for HFUS1, several challenges were met. In certain formulations, the molecule has a tendency to self-associate giving rise to risk of aggregation. The molecule has also shown tendency of AA clipping. The clipping happens on the chain B of the relaxin peptide while chain B is responsible for the binding of the molecule to its target. Furthermore, the molecule has high level of process-related HCPs and some species of HCPs present cause degradation to PS80 which led to high levels of particle formation of the molecule. This particle formation was not significantly influenced by the pH, buffer species and types of stabilising excipients but mainly driven by PS80 degradation. Extensive formulation development and optimisation studies were conducted. Arginine HCl was selected for its effectiveness in reducing the self-association tendency of the molecule and an optimised pH range was identified which effectively prevented AA clipping and fragmentation of the molecule. Detailed investigational work was conducted to understand the cause of the particle formation issue and PS80 was identified as the root cause. This led to the optimisation of the surfactant in the formulation system and alternative, lipase-resistant, surfactants such as P188 and TPGS mitigated the particle formation issue. P188 was selected as the lead surfactant for the formulation. A comprehensive formulation stability study was conducted to evaluate the robustness of the formulation system. The histidine-arginine HCl formulation showed excellent stability profile and robustness. The impact of HFUS1 concentration on stability was also evaluated at the end and HFUS1 showed good stability in the low concentration formulations (e.g. 0.25 mg/mL, 1 mg/mL, and 5 mg/mL), as well as higher concentration formulations (e.g. 33 mg/mL and 50 mg/mL).

1. A pharmaceutical formulation comprising a heterodimeric fusion and a lipase-resistant surfactant, wherein the heterodimeric fusion comprises:
(i) a first heterodimerisation domain connected to at least one Relaxin A chain polypeptide or a variant thereof; and
(ii) a second heterodimerisation domain connected to at least one Relaxin B chain polypeptide or a variant thereof,
wherein the first heterodimerisation domain heterodimerises with the second heterodimerisation domain, and wherein the heterodimeric fusion has Relaxin activity.

2. The pharmaceutical formulation according to claim 1, wherein the Relaxin A chain polypeptide and the Relaxin B chain polypeptide are covalently bound by at least one inter-chain disulphide bond.

3. The pharmaceutical formulation according to claim 1 or 2, wherein the Relaxin A chain and the Relaxin B chain are not covalently linked to each other by an amino acid linker.

4. The pharmaceutical formulation according to any one of the preceding claims, wherein the Relaxin A chain is a Relaxin-2 A chain and the Relaxin B chain is a Relaxin-2 B chain.

5. The pharmaceutical formulation according to any one of the preceding claims, wherein the Relaxin A chain is connected to the first heterodimerisation domain via a connector and the Relaxin B chain is connected to the second heterodimerisation domain via a connector, optionally wherein one or both connectors are polypeptides.

6. The pharmaceutical formulation according to claim 5, wherein one or both of the connectors have a length of between 6 and 40 amino acids, e.g. one or both connectors have a length of 21 amino acids.

7. The pharmaceutical formulation according to any one of the preceding claims, wherein the first and second heterodimerisation domains are derived from an immunoglobulin Fc region (“first Fc region” and “second Fc region”, respectively), optionally wherein the first and second Fc regions comprise the constant domains CH2 and CH3.

8. The pharmaceutical formulation according to claim 7, wherein the C-terminus of the first Fc region is connected to the N-terminus of the Relaxin A chain and the C-terminus of the second Fc region is connected to the N-terminus of the Relaxin B chain.

9. The pharmaceutical formulation according to claim 7 or 8, wherein the first and second Fc regions comprise heterodimerisation-promoting amino acid mutations and/or modifications, optionally wherein the heterodimerisation-promoting amino acid mutations are “Fc Knob” and “Fc Hole” mutations, e.g. “Fc Knob” and “Fc Hole” mutations present in the CH3 domains.

10. The pharmaceutical formulation according to any one of claims 7 to 9, wherein the first and second Fc regions are derived from a human IgG1 immunoglobulin.

11. The pharmaceutical formulation according to claim 10, wherein the heterodimerisation-promoting amino acid mutations comprise:
a. “Fc Hole” mutations Y349C, T366S, L368A and Y407V in one CH3 domain; and
b. “Fc Knob” mutations S354C and T366W in the other CH3 domain,
wherein the amino acid numbering is according to the EU index as in Kabat.

12. The pharmaceutical formulation according to claim 11, wherein:
a. the first Fc region comprises the “Fc Knob” mutations and the second Fc region comprises the “Fc Hole” mutations; or
b. the second Fc region comprises the “Fc Knob” mutations and the first Fc region comprises the “Fc Hole” mutations.

13. The pharmaceutical formulation according to any one of claims 10 to 12, wherein the first and/or second Fc region comprises the amino acid mutations L234F, L235E, and P331S, wherein the amino acid numbering is according to the EU index as in Kabat.

14. The pharmaceutical formulation according to any one of claims 4 to 13, wherein the Relaxin-2 A chain polypeptide comprises the sequence as set forth in SEQ ID NO: 1 or a variant thereof and the Relaxin-2 B chain polypeptide comprises the sequence as set forth in SEQ ID NO: 2 or a variant thereof.

15. The pharmaceutical formulation according to claim 14, wherein the Relaxin-2 A chain polypeptide comprises the amino acid mutation K9H, K17M or K171.

16. The pharmaceutical formulation according to any one of claims 5 to 15, wherein both connectors have the sequence GGGGSGGGGSGGGGSGGGGGS [SEQ ID NO: 5].

17. The pharmaceutical formulation according to claim 1, wherein the heterodimeric fusion comprises:
(i) an FcX-con-A fusion polypeptide; and
(ii) an FcY-con-B fusion polypeptide,
wherein:
A is a Relaxin A chain or variant thereof, e.g. a Relaxin-2 A chain or variant thereof;
B is a Relaxin B chain or variant thereof, e.g. Relaxin-2 B chain or variant thereof;
FcY is an Fc region comprising the constant domains CH2 and CH3 of a human IgG1 immunoglobulin and comprises “Fc Hole” amino acid mutations and/or modifications, optionally the amino acid mutations Y349C:T366S:L368A:Y407V;
FcX is an Fc region with “Fc Knob” amino acid mutations and/or modifications, optionally comprising the constant domains CH2 and CH3 of a human IgG1 immunoglobulin and comprises “Fc Knob” amino acid mutations and/or modifications, optionally the amino acid mutations S354C:T366W; and
con is a connector polypeptide, optionally having the sequence GGGGSGGGGSGGGGSGGGGGS [SEQ ID NO: 5],
wherein the amino acid numbering is according to the EU index as in Kabat, and wherein FcX heterodimerises with FcY.

18. The pharmaceutical formulation according to any one of the preceding claims, wherein the heterodimeric fusion comprises a fusion polypeptide with the amino acid sequence of SEQ ID NO: 11 and a fusion polypeptide with the amino acid sequence of SEQ ID NO: 20.

19. The pharmaceutical formulation according to any one of claims 8 to 18, wherein the heterodimeric fusion further comprises one or more Fabs, optionally wherein the heterodimeric fusion comprises one Fab linked to the N-terminus of the first Fc region and a second Fab linked to the N-terminus of the second Fc region.

20. The pharmaceutical formulation according to any one of the claims 8 to 19, wherein the heterodimer further comprises a second Relaxin A chain polypeptide or variant thereof connected to the N-terminus of the first Fc region and a second Relaxin B chain polypeptide or variant thereof connected to the N-terminus of the second Fc region, optionally wherein the second Relaxin A chain is connected to the first Fc region via a connector polypeptide and the second Relaxin B chain is connected to the second Fc region via a connector polypeptide.

21. The pharmaceutical formulation according to claim 1, wherein the heterodimeric fusion comprises:
(i) FcX-B-L-A and FcY, optionally FcY-B-L-A; or
(ii) FcY-B-L-A and FcX, optionally FcX-B-L-A;
wherein:
FcY is an immunoglobulin Fc region with “Fc Hole” amino acid mutations and/or modifications, optionally comprising a CH3 domain having the amino acid mutations Y349C:T366S:L368A:Y407V;
FcX is an immunoglobulin Fc region with “Fc Knob” amino acid mutations and/or modifications, optionally comprising a CH3 domain having the amino acid mutations S354C:T366W;
B is a Relaxin B chain or a variant thereof, e.g. a Relaxin 2 B chain or variant thereof;
A is a Relaxin A chain or a variant thereof, e.g. a Relaxin 2 A chain or variant thereof; and
L is a linker polypeptide, optionally having the amino acid sequence GGGSGGGSGG [SEQ ID NO: 60],
wherein the amino acid numbering is according to the EU index as in Kabat, and wherein FcX heterodimerises with FcY.

22. The pharmaceutical formulation according to claim 21, wherein the Relaxin B chain is connected to FcX and/or FcY via a connector, optionally a connector polypeptide having a length of between 6 and 40 amino acids, e.g. a length of 21 amino acids.

23. The pharmaceutical formulation of any of the preceding claims, wherein the formulation comprises less than about 10,000, about 6000, about 5,000, about 1,000, about 750, about 600, about 500, about 250, about 150, about 100, or about 50 particles/mL greater than 2 μm, 5 μm, 10 μm, 15 μm, 20 μm or 25 μm diameter.

24. The pharmaceutical formulation according to any one of the preceding claims, wherein the concentration of the lipase-resistant surfactant is from 0.001% (w/v) to 1% (w/v).

25. The pharmaceutical formulation according to any one of the preceding claims, wherein the concentration of the lipase-resistant surfactant is from 0.02% (w/v) to 0.06% (w/v), optionally 0.04% (w/v).

26. The pharmaceutical formulation according to any one of the preceding claims, wherein the lipase-resistant surfactant cannot be enzymatically hydrolysed by lipase, optionally wherein the lipase is selected from lipoprotein lipase, Lipase 9, Phospholipase 2 and Phospholipase 2A.

27. The pharmaceutical formulation according to any one of the preceding claims, wherein the lipase-resistant surfactant does not comprise an ester bond capable of being enzymatically hydrolysed by lipase, optionally wherein the lipase is selected from lipoprotein lipase, Lipase 9, Phospholipase 2 and Phospholipase 2A.

28. The pharmaceutical formulation according to any one of the preceding claims, wherein the lipase-resistant surfactant is a water-soluble non-ionic triblock copolymer formed by polyethylene oxide (PEO) and polypropylene oxide (PPO) blocks, optionally wherein the water-soluble nonionic triblock copolymer is poloxamer 188 (P188).

29. The pharmaceutical formulation according to any one of claims 1 to 27, wherein the lipase-resistant surfactant is selected from P188, D-α-Tocopherol polyethylene glycol succinate (TPGS), Kolliphor HS15, Kolliphor EL, Kolliphor RH40, PEG 300, PEG400, Brij 58 and Brij 35; optionally wherein the lipase-resistant surfactant is TPGS.

30. The pharmaceutical formulation according to any one of the preceding claims, wherein the formulation further comprises a buffer at a pH from about 3 to about 10, optionally about 5.5 to about 7.5.

31. The pharmaceutical formulation according to claim 30, wherein the formulation has a pH in the range of 6 to 7.

32. The pharmaceutical formulation according to claim 31, wherein the formulation has a pH of 6.5.

33. The pharmaceutical formulation according to any one of claims 30 to 32, wherein the concentration of the buffer is from 0.1 mM to 100 mM, optionally 5 mM, 10 mM, 15 mM, 20 mM, 25 mM, 30 mM, 35 mM, 40 mM, 45 mM, or 50 mM.

34. The pharmaceutical formulation according to claim 33, wherein the concentration of the buffer is 10 mM to 30 mM, optionally 20 mM.

35. The pharmaceutical formulation according to any one of claims 30 to 34, wherein the buffer is selected from acetate, acetic acid, succinate, succinic acid, phosphate, phosphoric acid, ascorbate, ascorbic acid, lactate, lactic acid, tartartic acid, maleic acid, glycine, gluconate, citrate, histidine, imidazole, bicarbonate and carbonic acid, sodium benzoate, benzoic acid, edetate, malate, tris, glycylglycine and mixtures thereof, optionally wherein the buffer is selected from a citrate buffer and a histidine buffer.

36. The pharmaceutical formulation according to any one of claims 30 to 35, wherein the buffer is a histidine, histidine hydrochloride or histidine/histidine hydrochloride buffer, optionally a histidine/histidine hydrochloride buffer.

37. The pharmaceutical formulation according to any one of the preceding claims, wherein the formulation additionally comprises an excipient, optionally wherein the excipient is an ionic excipient.

38. The pharmaceutical formulation according to claim 37, wherein the concentration of the excipient is from 10 mM to 500 mM.

39. The pharmaceutical formulation according to claim 38, wherein the concentration of the excipient is from 140 mM to 240 mM, optionally 190 mM.

40. The pharmaceutical formulation according to any one of claims 37 to 39, wherein the excipient is an ionic excipient selected from an arginine salt or a lysine salt.

41. The pharmaceutical formulation according to claim 40, wherein the ionic excipient is selected from arginine HCl or lysine HCl, optionally arginine HCl.

42. The pharmaceutical formulation according to any one of the preceding claims, wherein the formulation further comprises a sugar, optionally wherein the sugar is sucrose.

43. The pharmaceutical formulation according to any one of the preceding claims, wherein the concentration of heterodimeric fusion is from 0.1 to 100 mg/mL, optionally 0.2 to 50 mg/mL.

44. The pharmaceutical formulation according to claim 43, wherein the formulation comprises 0.2-50 mg/mL of heterodimeric fusion, 20 mM histidine/histidine hydrochloride buffer, 190 mM arginine HCl, 0.04% (w/v) poloxamer 188 (P188), and wherein the formulation has a pH of 6.5.

45. The pharmaceutical formulation according to claim 44, wherein the heterodimeric fusion consists of a fusion polypeptide with the amino acid sequence of SEQ ID NO: 11 and a fusion polypeptide with the amino acid sequence of SEQ ID NO: 20.

46. The pharmaceutical formulation according to any one of the preceding claims, wherein the formulation comprises 50 mg/ml of heterodimeric fusion.

47. The pharmaceutical formulation according to any one of claims 1 to 45, wherein the formulation comprises 30 mg/ml of heterodimeric fusion.

48. The pharmaceutical formulation according to any one of the claims 1 to 45, wherein the formulation comprises 5 mg/ml or 1.1 mg/ml or 1 mg/ml of heterodimeric fusion.

49. The pharmaceutical formulation according to any one of the preceding claims for use in therapy.

50. The pharmaceutical formulation according to any one of claims 1 to 48 for use in the treatment of a subject with heart failure with pulmonary hypertension.

51. The pharmaceutical formulation for use according to claim 49 or claim 50, wherein the pharmaceutical formulation is administered to the subject by subcutaneous injection.

52. The pharmaceutical formulation for use according to any one of claims 49 to 51, wherein the fusion polypeptide or pharmaceutical formulation is administered by self-administration.

53. The pharmaceutical formulation for use according to any one of claims 50 to 52, wherein the heart failure is heart failure with reduced ejection fraction, heart failure with mid-range ejection fraction or heart failure with preserved ejection fraction.

54. The pharmaceutical formulation for use according to any one of claims 50 to 53, wherein the subject has a mean Pulmonary Arterial Pressure of about 25 mmHg or greater, a pulmonary artery wedge pressure (PAWP) greater than 15 mmHg and/or a Right Ventricular Systolic Pressure of about 40 mmHg or greater.

55. The pharmaceutical formulation for use according to any one of claims 50 to 54, wherein the subject has a Pulmonary Vascular Resistance of less than 3.0 wood units.

56. The pharmaceutical formulation for use according to any one of claims 50 to 54, wherein the subject has a Pulmonary Vascular Resistance of 3.0 or more wood units.

57. The pharmaceutical formulation for use according to any one of claims 50 to 56, wherein the subject has been fitted with a blood pressure monitoring device, optionally a pulmonary artery pressure monitoring device.

58. The pharmaceutical formulation for use according to claim 57, wherein the pulmonary artery pressure monitoring device is a CardioMEMS pressure monitoring device.

59. The pharmaceutical formulation for use according to any one of claims 49 to 58, wherein administration of the pharmaceutical formulation results in one or more of:
a) reduced PVR;
(b) reduced mPAP;
(c) reduced ePAD;
(d) increased stroke volume (SV) of the heart;
(e) decreased systemic vascular resistance (SVR) and/or increase estimated glomerular filtration rate (eGFR);
(f) increased ejection fraction; and/or
(g) increased cardiac output;
as compared to baseline levels pre-administration.

60. A kit comprising the pharmaceutical formulation according to any one of claims 1 to 48.

61. A method of treating a subject with a disease or disorder, the method comprising administering the pharmaceutical formulation according to any one of claims 1 to 48 to the subject.

62. A method of treating a subject with heart failure with pulmonary hypertension, the method comprising administering the pharmaceutical formulation according to any one of claims 1 to 48 to the subject.

63. The method according to claim 61 or claim 62, wherein the heterodimeric fusion or pharmaceutical formulation is administered to the subject by subcutaneous injection.

64. The method according to any one of claims 61 to 63, wherein the heterodimeric fusion or pharmaceutical formulation is administered by self-administration.

65. The method according to any one of claims 62 to 64, wherein the heart failure is heart failure with reduced ejection fraction, heart failure with mid-range ejection fraction or heart failure with preserved ejection fraction.

66. The method according to any one of claims 62 to 65, wherein the subject has a mean Pulmonary Arterial Pressure of about 25 mmHg or greater, a pulmonary artery wedge pressure (PAWP) greater than 15 mmHg and/or a Right Ventricular Systolic Pressure of about 40 mmHg or greater.

67. The method according to any one of claims 62 to 66, wherein the subject has a Pulmonary Vascular Resistance of less than 3.0 wood units.

68. The method according to any one of claims 62 to 66, wherein the subject has a Pulmonary Vascular Resistance of 3.0 or more wood units.

69. The method according to any one of claims 62 to 68, wherein the subject has been fitted with a blood pressure monitoring device, optionally a pulmonary artery pressure monitoring device.

70. The method according to any one of claims 62 to 69, wherein the pulmonary artery pressure monitoring device is a CardioMEMS pressure monitoring device.

71. The method according to any one of claims 61 to 70, wherein administration of the heterodimeric fusion or pharmaceutical formulation results in one or more of:
a) reduced PVR;
(b) reduced mPAP;
(c) reduced ePAD;
(d) increased stroke volume (SV) of the heart;
(e) decreased systemic vascular resistance (SVR) and/or increase estimated glomerular filtration rate (eGFR);
(f) increased ejection fraction; and/or
(g) increased cardiac output;
as compared to baseline levels pre-administration.


SEQUENCE LISTING (74 sequences)

SEQ ID NO: 1 - protein, 24 aa, Homo sapiens
QLYSALANKCCHVGCTKRSLARFC

SEQ ID NO: 2 - protein, 28 aa, Homo sapiens
  REGION 1..28
SWMEEVIKLCGRELVRAQIAICGMSTWS

SEQ ID NO: 3 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 4 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 21 aa, synthetic construct
GGGGSGGGGSGGGGSGGGGGS

SEQ ID NO: 6 - protein, 21 aa, synthetic construct
PAPAPAPAPAPAPAPAPAPAG

SEQ ID NO: 7 - protein, 312 aa, synthetic construct
SWMEEVIKLCGRELVRAQIAICGMSTWSGGGGSGGGGSGGGGSQLYSALANKCCHVGCTKRSLARFCAAAGGGGSGGGGSGGGGSGGGGSACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 309 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLYSALANKCCHVGCTKRSLARFCGGGGSGGGGSGGGGSSWMEEVIKLCGRELVRAQIAICGMSTWS

SEQ ID NO: 9 - protein, 272 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 10 - protein, 272 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 11 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 12 - protein, 272 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 13 - protein, 272 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 14 - protein, 275 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 15 - protein, 276 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 16 - protein, 276 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 17 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 18 - protein, 276 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 19 - protein, 276 aa, synthetic construct
GGAGGACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 20 - protein, 275 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 21 - protein, 266 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGSQLYSALANKCCHVGCTKRSLARFC

SEQ ID NO: 22 - protein, 270 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGGSSWMEEVIKLCGRELVRAQIAICGMSTWS

SEQ ID NO: 23 - protein, 261 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGGSQLYSALANKCCHVGCTKRSLARFC

SEQ ID NO: 24 - protein, 265 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGGSSWMEEVIKLCGRELVRAQIAICGMSTWS

SEQ ID NO: 25 - protein, 256 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGSQLYSALANKCCHVGCTKRSLARFC

SEQ ID NO: 26 - protein, 260 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGSSWMEEVIKLCGRELVRAQIAICGMSTWS

SEQ ID NO: 27 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAPAPAPAPAPAPAPAPAPAGSQLYSALANKCCHVGCTKRSLARFC

SEQ ID NO: 28 - protein, 275 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAPAPAPAPAPAPAPAPAPAGSSWMEEVIKLCGRELVRAQIAICGMSTWS

SEQ ID NO: 29 - protein, 266 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAAPAPAPAPAPAPAGSQLYSALANKCCHVGCTKRSLARFC

SEQ ID NO: 30 - protein, 270 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAAPAPAPAPAPAPAGSSWMEEVIKLCGRELVRAQIAICGMSTWS

SEQ ID NO: 31 - protein, 261 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAPAPAPAPAGSQLYSALANKCCHVGCTKRSLARFC

SEQ ID NO: 32 - protein, 265 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAPAPAPAPAGSSWMEEVIKLCGRELVRAQIAICGMSTWS

SEQ ID NO: 33 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANECCHVGCTKRSLARFC

SEQ ID NO: 34 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANHCCHVGCTKRSLARFC

SEQ ID NO: 35 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANLCCHVGCTKRSLARFC

SEQ ID NO: 36 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANMCCHVGCTKRSLARFC

SEQ ID NO: 37 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKESLARFC

SEQ ID NO: 38 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKHSLARFC

SEQ ID NO: 39 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAAFC

SEQ ID NO: 40 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAIFC

SEQ ID NO: 41 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAMFC

SEQ ID NO: 42 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAQFC

SEQ ID NO: 43 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLASFC

SEQ ID NO: 44 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAREC

SEQ ID NO: 45 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARIC

SEQ ID NO: 46 - protein, 308 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSSWMEEVIKLCGRELVRAQIAICGMSTWSGGGSGGGSGQLYSALANKCCHVGCTKRSLARFC

SEQ ID NO: 47 - protein, 308 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWSGGGSGGGSGQLYSALANKCCHVGCTKRSLARFC

SEQ ID NO: 48 - protein, 273 aa, synthetic construct
SWMEEVIKLCGRELVRAQIAICGMSTWSAAAGGGGSGGGGSGGGGSGGGGSACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 49 - protein, 269 aa, synthetic construct
QLYSALANKCCHVGCTKRSLARFCAAAGGGGSGGGGSGGGGSGGGGSACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 314 aa, synthetic construct
QLYSALANKCCHVGCTKRSLARFCAAAGGGGSGGGGSGGGGSGGGGSACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 51 - protein, 322 aa, synthetic construct
SWMEEVIKLCGRELVRAQIAICGMSTWSAAAGGGGSGGGGSGGGGSGGGGSACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 52 - protein, 273 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMST

SEQ ID NO: 53 - protein, 272 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMS

SEQ ID NO: 54 - protein, 213 aa, synthetic construct
ELVLTQPASVSGSPGQSITISCTGTSSDVGGYNYVSWYQQHPGKAPKLMIYDVSKRPSGVSNRFSGSKSGNTASLTISGQAEDEADYYCSSYTSSSTLVFGGGTKLTVLGQPKAAPSVTLFPPSSEELQANKATLVCLISDFYPGAVTVAWKADSSPVAGVETTTPSKQSNNKYAASSYLSLTPEQWKSHRSYSCQTHEGSTVEKTVAPTECS

SEQ ID NO: 55 - protein, 494 aa, synthetic construct
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARFC

SEQ ID NO: 56 - protein, 498 aa, synthetic construct
EVQLLESGGGLVQPGGSLRLSCTTSGFTFNTYAMSWVRQAPGKGLEWLSGINNNGRTAFYADSVKGRFTISRDNSKNTLYLQINSLRADDTAVYFCAKDVRFIAVPGDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 57 - protein, 292 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSPQLYSALANKCCHVGCTKRSLARFCGGGSGGGSGSWMEEVIKLCGRELVRAQIAICGMSTWS

SEQ ID NO: 58 - protein, 295 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSPQLYSALANKCCHVGCTKRSLARFCGGGSGGGSGSWMEEVIKLCGRELVRAQIAICGMSTWS

SEQ ID NO: 59 - protein, 298 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSPQLYSALANKCCHVGCTKRSLARFCGGGSGGGSGSWMEEVIKLCGRELVRAQIAICGMSTWS

SEQ ID NO: 60 - protein, 10 aa, synthetic construct
GGGSGGGSGG

SEQ ID NO: 61 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLARAC

SEQ ID NO: 62 - protein, 29 aa, synthetic construct
DSWMEEVIKLCGRELVRAQIAICGMSTWS

SEQ ID NO: 63 - protein, 24 aa, synthetic construct
QLYSALANKCCHVGCTKRSLARFC

SEQ ID NO: 64 - protein, 33 aa, synthetic construct
MRVSEEWMDGFIRMCGREYARELIKICGASVGR

SEQ ID NO: 65 - protein, 25 aa, synthetic construct
ESGGLMSQQCCHVGCSRRSIAKLYC

SEQ ID NO: 66 - protein, 272 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCRVGCTKRSLARFC

SEQ ID NO: 67 - protein, 276 aa, synthetic construct
DKTHTACPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGGSSWMEEVIKLCGRELVRAQIAICGMSTWS

SEQ ID NO: 68 - protein, 271 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGSQLYSALANKCCHVGCTKRSLAYFC

SEQ ID NO: 69 - protein, 9 aa, synthetic construct
DKTHTCPPC

SEQ ID NO: 70 - protein, 10 aa, synthetic construct
DKTHTACPPC

SEQ ID NO: 71 - protein, 10 aa, synthetic construct
GGAGGACPPC

SEQ ID NO: 72 - protein, 5 aa, synthetic construct
ACPPC

SEQ ID NO: 73 - protein, 4 aa, synthetic construct
  REGION >4: note = Amino acids 3-4 of the sequence can be repeated one or more times.
PAPA

SEQ ID NO: 74 - protein, 5 aa, synthetic construct
  REGION >5: note = The entire sequence of amino acids 1-5 can be repeated 1- 7 times.
GGGGS